(19) United States
(12) Patent Application Publication
(10) Pub. No.: US 2009/0099184 A1
(43) Pub. Date: Apr. 16, 2009

(54) Substituted pyridineamide compounds useful as soluble epoxide hydrolase inhibitors

(75) Inventors: Stephane Delombaert, Madison, CT (US); Anne Bettina Eldrup, Danbury, CT (US); Neil Alexander Farrow, Ridgefield, CT (US); Jennifer A. Kowalski, New Milford, CT (US); Ingo Andreas Mugge, New Haven, CT (US); Fariba Soleymanzadeh, Danbury, CT (US); Alan David Swinamer, Southbury, CT (US); Steve John Taylor, Southbury, CT (US)

Correspondence Address: Micheal P. Morris;Boehringer Ingelheim USA Corporation, 900 Ridgebury Road, Ridgefield, CT, 06877-0368, US

(73) Assignee: BOEHRINGER INGELHEIM INTERNATIONAL GMBH, Ingelheim (DE)

(21) Appl. No.: 12/278,063

(22) PCT Filed: Feb. 15, 2007
(86) PCT No.: PCT/US2007/062168

§ 371 Date: Aug. 1, 2008

Related U.S. Application Data
(60) Provisional application No. 60/743,301, filed on Feb. 16, 2006.

(51) Int. Cl. A61K 31/5377; C07D 213/78; A61K 31/44; C07D 213/62; A61P 9/12; A61P 13/12; A61P 9/00; A61P 3/10; A61K 31/444; C07D 413/02

(52) U.S. Cl. 514/237.2; 546/298; 514/350; 546/261; 514/335; 544/124

(57) ABSTRACT

Disclosed are compounds active against soluble epoxide hydrolase (sEH), compositions thereof and methods of using and making same.

### APPLICATION DATA

This application claims benefit to U.S. provisional application 60/743,301 filed Feb. 16, 2006.

### BACKGROUND OF THE INVENTION

1. Technical Field

This invention relates to compounds possessing anti-sEH activity and methods of using soluble epoxide hydrolase (sEH) inhibitors for diseases related to cardiovascular disease.

2. Background Information

Epoxide hydrolases are a group of enzymes ubiquitous in nature, detected in species ranging from plants to mammals. These enzymes are functionally related in that they all catalyze the addition of water to an epoxide, resulting in a diol. Epoxide hydrolases are important metabolizing enzymes in living systems and their diol products are frequently found as intermediates in the metabolic pathway of xenobiotics. Epoxide hydrolases are therefore important enzymes for the detoxification of epoxides by conversion to their corresponding, non-reactive diols.

In mammals, several types of epoxide hydrolases have been characterized including soluble epoxide hydrolase (sEH), also referred to as cytosolic epoxide hydrolase, cholesterol epoxide hydrolase, LTA₄ hydrolase, hepoxilin hydrolase, and microsomal epoxide hydrolase (Fretland and Omiecinski, Chemico-Biological Interactions, 129: 41-59 (2000)). Epoxide hydrolases have been found in all tissues examined in vertebrates including heart, kidney and liver (Vogel, et al., Eur J. Biochemistry, 126: 425-431 (1982); Schladt et al., Biochem. Pharmacol., 35: 3309-3316 (1986)). Epoxide hydrolases have also been detected in human blood components including lymphocytes (e.g. T-lymphocytes), monocytes, erythrocytes, platelets and plasma. In the blood, most of the sEH detected was present in lymphocytes (Seidegard et al., Cancer Research, 44: 3654-3660 (1984)).

The epoxide hydrolases differ in their specificity towards epoxide substrates. For example, sEH is selective for aliphatic epoxides such as epoxide fatty acids while microsomal epoxide hydrolase (mEH) is more selective for cyclic and arene epoxides. The primary known physiological substrates of sEH are four regioisomeric cis epoxides of arachidonic acid, 5,6-, 8,9-, 11,12-, and 14,15-epoxyeicosatrienoic acid, also known as epoxyeicosatrienoic acids or EETs. Also known to be substrates for sEH are epoxides of linoleic acid known as leukotoxin or isoleukotoxin. Both the EETs and the leukotoxins are generated by members of the cytochrome P450 monooxygenase family (Capdevila, et al., J. Lipid Res., 41: 163-181 (2000)).

EETs function as chemical autocrine and paracrine mediators in the cardiovascular and renal systems (Spector, et al, Progress in Lipid Research, 43: 55-90 (2004); Newman, et al., Progress in Lipid Research 44: 1-51 (2005)). EETs appear to be able to function as endothelial derived hyperpolarizing factor (EDHF) in various vascular beds due to their ability to cause hyperpolarization of the membranes of vascular smooth muscle cells with resultant vasodilation (Weintraub, et al., Circ. Res., 81: 258-267 (1997)). EDHF is synthesized from arachidonic acid by various cytochrome P450 enzymes in endothelial cells proximal to vascular smooth muscle (Quilley, et al., Brit. Pharm., 54: 1059 (1997); Quilley and McGiff, TIPS, 21: 121-124 (2000)); Fleming and Busse, Nephrol. Dial. Transplant, 13: 2721-2723 (1998)). In the vascular smooth muscle cells EETs provoke signaling pathways which lead to activation of BK_{Ca2+} channels (big Ca²⁺ activated potassium channels) and inhibition of L-type Ca²⁺ channels, ultimately resulting in hyperpolarization of membrane potential, inhibition of Ca²⁺ influx and relaxation (Li et al., Circ. Res., 85: 349-356 (1999)). Endothelium dependent vasodilation has been shown to be impaired in different forms of experimental hypertension as well as in human hypertension (Lind, et al., Blood Pressure, 9: 4-15 (2000)). Impaired endothelium dependent vasorelaxation is also a characteristic feature of the syndrome known as endothelial dysfunction (Goligorsky, et. al., Hypertension, 37[part 2]:744-748 (2001)). Endothelial dysfunction plays a significant role in a large number of pathological conditions including type 1 and type 2 diabetes, insulin resistance syndrome, hypertension, atherosclerosis, coronary artery disease, angina, ischemia, ischemic stroke, Raynaud's disease and renal disease. Hence, it is likely that enhancement of EETs concentration would have a beneficial therapeutic effect in patients where endothelial dysfunction plays a causative role. Other effects of EETs that may influence hypertension involve effects on kidney function. Levels of various EETs and their hydrolysis products, the DHETs, increase significantly both in the kidneys of spontaneously hypertensive rats (SHR) (Yu, et al., Circ. Res. 87: 992-998 (2000)) and in women suffering from pregnancy induced hypertension (Catella, et al., Proc. Natl. Acad. Sci. U.S.A., 87: 5893-5897 (1990)). In angiotensin II infused rats the treatment with a selective sEH inhibitor attenuated the afferent arteriolar diameter in the kidney and lowered urinary albumin secretion, a marker of compromised renal function, suggesting antihypertensive and renal vascular protective effects of increased EETs levels (Zhao, et al, 15: 1244-1253 (2004)). In the spontaneously hypertensive rat model, both cytochrome P450 and sEH activities were found to increase (Yu et al., Molecular Pharmacology, 57: 1011-1020 (2000)). Addition of a known sEH inhibitor was shown to decrease the blood pressure to normal levels. Furthermore, administration of a selective sEH inhibitor to angiotensin II treated rats was demonstrated to lower systolic blood pressure (Imig, et al, Hypertension, 39: 690-694 (2002)). Finally, male soluble epoxide hydrolase null mice exhibited a phenotype characterized by lower blood pressure than their wild-type counterparts (Sinal, et al., J. Biol. Chem., 275: 40504-40510 (2000)).

EETs, especially 11,12-EET, also have been shown to exhibit anti-inflammatory properties (Node, et al., Science, 285: 1276-1279 (1999); Campbell, TIPS, 21: 125-127 (2000); Zeldin and Liao, TIPS, 21: 127-128 (2000)). Node, et al. have demonstrated 11,12-EET decreases expression of cytokine induced endothelial cell adhesion molecules, especially VCAM-1. They further showed that EETs prevent leukocyte adhesion to the vascular wall and that the mechanism responsible involves inhibition of NF-κB and IκB kinase. Vascular inflammation plays a role in endothelial dysfunction (Kessler, et al., Circulation, 99: 1878-1884 (1999)). Hence, the ability of EETs to inhibit the NF-κB pathway should also help ameliorate this condition. In addition, the administration of EETs and/or the administration of a selective sEH inhibitor was demonstrated to attenuate tobacco smoke induced inflammation, as assessed total bronchoalveolar lavage cell numbers and concomittant reduction in neutrophils, alveolar macrophages, and lymphocytes (Smith, et al, 102: 2186-2191 (2005)).

In addition to the physiological effect of some substrates of sEH (EETs, mentioned above), some diols, i.e. DHETs, produced by sEH may have potent biological effects. For example, sEH metabolism of epoxides produced from linoleic acid (leukotoxin and isoleukotoxin) produces leukotoxin and isoleukotoxin diols (Greene, et al., Arch. Biochem. Biophys. 376(2): 420-432 (2000)). These diols were shown to be toxic to cultured rat alveolar epithelial cells, increasing intracellular calcium levels, increasing intercellular junction permeability and promoting loss of epithelial integrity (Moghaddam et al., Nature Medicine, 3: 562-566 (1997)). Therefore these diols could contribute to the etiology of diseases such as adult respiratory distress syndrome where lung leukotoxin levels have been shown to be elevated (Ishizaki, et al., Pulm. Pharm. & Therap., 12: 145-155 (1999)). Hammock, et al. have disclosed the treatment of inflammatory diseases, in particular adult respiratory distress syndrome and other acute inflammatory conditions mediated by lipid metabolites, by the administration of inhibitors of epoxide hydrolase (WO 98/06261; U.S. Pat. No. 5,955,496).

A number of classes of sEH inhibitors have been identified. Among these are chalcone oxide derivatives (Miyamoto, et al. Arch. Biochem. Biophys., 254: 203-213 (1987)) and various trans-3-phenylglycidols (Dietze, et al., Biochem. Pharm. 42: 1163-1175 (1991); Dietze, et al., Comp. Biochem. Physiol. B, 104: 309-314 (1993)).

More recently, Hammock et al. have disclosed certain biologically stable inhibitors of sEH for the treatment of inflammatory diseases, for use in affinity separations of epoxide hydrolases and in agricultural applications (U.S. Pat. No. 6,150,415). The Hammock '415 patent also generally describes that the disclosed pharmacophores can be used to deliver a reactive functionality to the catalytic site, e.g., alkylating agents or Michael acceptors, and that these reactive functionalities can be used to deliver fluorescent or affinity labels to the enzyme active site for enzyme detection (col. 4, line 66 to col. 5, line 5). Certain urea and carbamate inhibitors of sEH have also been described in the literature (Morisseau et al., Proc. Natl. Acad. Sci., 96: 8849-8854 (1999); Argiriadi et al., J. Biol. Chem., 275 (20): 15265-15270 (2000); Nakagawa et al. Bioorg. Med. Chem., 8: 2663-2673 (2000); US 2005/0026844 and Kim, et al., J. Med. Chem. 47(8): 2110-2122 (2004) both of which describe inhibitors with additional, tethered oxo pharmacophores).

WO 00/23060 discloses a method of treating immunological disorders mediated by T-lymphocytes by administration of an inhibitor of sEH. Several 1-(4-aminophenyl)pyrazoles are given as examples of inhibitors of sEH.

U.S. Pat. No. 6,150,415 to Hammock is directed to a method of inhibiting an epoxide hydrolase, using compounds having the structure

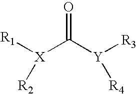

wherein X and Y is each independently nitrogen, oxygen, or sulfur, and X can further be carbon, at least one of R1-R4 is hydrogen, R2 is hydrogen when X is nitrogen but is not present when X is sulfur or oxygen, R4 is hydrogen when Y is nitrogen but is not present when Y is sulfur or oxygen, R1 and R3 is each independently H, C1-20 substituted or unsubstituted alkyl, cycloalkyl, aryl, acyl, or heterocyclic. Related to the Hammock patent is U.S. Pat. No. 6,531,506 to Kroetz et al. which claims a method of treating hypertension using of an inhibitor of epoxide hydrolase, also claimed are methods of treating hypertension using compounds similar to those described in the Hammock patent. Neither of these patents teaches or suggests methods of treating cardiovascular diseases using the particular sEH inhibitors described herein.

As outlined in the discussion above, inhibitors of sEH are useful therefore, in the treatment of cardiovascular diseases such as endothelial dysfunction either by preventing the degradation of sEH substrates that have beneficial effects or by preventing the formation of metabolites that have adverse effects.

All references cited above and throughout this application are incorporated herein by reference in their entirety.

### BRIEF SUMMARY OF THE INVENTION

It is therefore an object of the invention to provide compounds active as sEH inhibitors of the formula I as described herein below.

It is a further object of the invention to provide a method of treating hypertension by administering to a patient a compound of the formula I as described herein below. It is yet a further object to provide methods of making the compounds described herein below.

### DETAILED DESCRIPTION OF THE INVENTION

In one generic aspect of the invention, there is provided a compound of the formula (I):

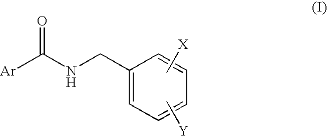

Wherein:

Ar is phenyl or pyridinyl each optionally substituted with one to three substituent groups chosen from C₁₋₄ alkyl, C₃₋₆ cycloalkyl, C₂₋₄ alkynyl, C₁₋₄ alkyloxycarbonyl, C₁₋₄ alkylamidocarbonyl, C₁₋₄ dialkylamidocarbonyl, C₁₋₄ alkoxy, C₃₋₆ cycloalkylamino, di(C₃₋₆ cyclo)alkylamino, C₁₋₄ alkylsulfonyl, C₁₋₄ alkylheterocyclyl, halogen, hydroxyl, phenyl, naphthyl, heterocyclyl and heteroaryl
wherein each substituent group is optionally independently substituted with one to three substituents chosen from amino, cyano, carboxy, carboxamido, halogen, hydroxyl, sulfonyl, sulfonamide, C₁₋₄ alkyl, C₃₋₆ cycloalkyl, C₂₋₄ alkynyl, C₁₋₄ alkyloxycarbonyl, C₁₋₄ alkylamidocarbonyl, C₁₋₄ dialkylamidocarbonyl, C₁₋₄ alkoxy, C₃₋₆ cycloalkylamino, di(C₃₋₆ cyclo)alkylamino, C₁₋₄ alkylsulfonyl, C₁₋₄ alkylheterocyclyl, phenyl or pyridinyl optionally substituted with one to three substituents chosen from amino, cyano, halogen, hydroxyl and C₁₋₄ alkylsulfonyl, C₁₋₆ alkyl optionally substituted with hydroxyl, amino, C₁₋₄ alkoxy, C₁₋₄ alkylamino, C₁₋₄ alkylthio, or one to three fluorine atoms.

X and Y are optionally independently hydrogen, halogen, cyano, NH—R, OR, R, SO₂R or S(O)₂NRR, wherein R is independently hydrogen, aryl or C₁₋₆ alkyl optionally substituted with hydroxyl, amino, C₁₋₄ alkoxy, C₁₋₄ alkylamino, C₁₋₄ alkylthio, or one to three fluorine atoms.

And wherein both X and Y are not hydrogen

or the pharmaceutically acceptable salts thereof.

In another embodiment there is provided a compound according to the embodiment immediately above and wherein:

Ar is pyridinyl optionally substituted with one to three substituent groups chosen from C₁₋₄ alkyl, C₃₋₆ cycloalkyl, C₂₋₄ alkynyl, C₁₋₄ alkyloxycarbonyl, C₁₋₄ alkylamidocarbonyl, C₁₋₄ dialkylamidocarbonyl, C₁₋₄ alkoxy, C₃₋₆ cycloalkylamino, di(C₃₋₆ cyclo)alkylamino, C₁₋₄ alkylsulfonyl, C₁₋₄ alkylheterocyclyl, halogen, hydroxyl, pyridinyl, pyrazinyl, pyridazinyl, quinolinyl and isoquinolinyl
wherein each substituent group is optionally independently substituted with one to three substituents chosen from amino, cyano, carboxy, carboxamido, halogen, hydroxyl, sulfonyl, sulfonamide, C₁₋₄ alkylsulfonyl, C₁₋₄ alkylheterocyclyl, phenyl or pyridinyl optionally substituted with one to three substituents chosen from amino, cyano, halogen, hydroxyl and C₁₋₄ alkylsulfonyl, C₁₋₆ alkyl optionally substituted with hydroxyl, amino, C₁₋₄ alkoxy, C₁₋₄ alkylamino, C₁₋₄ alkylthio, or one to three fluorine atoms.

X and Y are optionally independently hydrogen, halogen, cyano, NH—R, OR, R, SO₂R, wherein R is independently hydrogen, aryl optionally substituted with one to three substituents chosen from amino, cyano, halogen, hydroxyl and C₁₋₄ alkylsulfonyl, C₁₋₆ alkyl optionally substituted with hydroxyl, amino, C₁₋₄ alkoxy, C₁₋₄ alkylamino, C₁₋₄ alkylthio, or one to three fluorine atoms.

And wherein both X and Y are not hydrogen

In another embodiment there is provided a compound according to the embodiment immediately above and wherein:

Ar is pyridinyl optionally substituted with one to three substituent groups chosen from C₁₋₄ alkyl, C₁₋₄ alkoxy, C₃₋₆ cycloalkylamino, di(C₃₋₆ cyclo)alkylamino, C₁₋₄ alkylsulfonyl, C₁₋₄ alkylheterocyclyl, halogen and hydroxyl
wherein each substituent group is optionally independently substituted with one to three substituents chosen from amino, cyano, carboxy, carboxamido, halogen, hydroxyl, sulfonyl, sulfonamide, C₁₋₄ alkylsulfonyl, C₁₋₄ alkylheterocyclyl, phenyl or pyridinyl optionally substituted with one to three substituents chosen from amino, cyano, halogen, hydroxyl and C₁₋₄ alkylsulfonyl, C₁₋₆ alkyl optionally substituted with hydroxyl, amino, C₁₋₄ alkoxy, C₁₋₄ alkylamino, C₁₋₄ alkylthio, or one to three fluorine atoms.

X and Y are optionally independently hydrogen, chloro, bromo, cyano, OR, SO₂R, wherein R is independently hydrogen, C₁₋₄ alkyl or aryl optionally substituted with one to three substituents chosen from amino, cyano, halogen, hydroxyl and C₁₋₄ alkylsulfonyl

And wherein both X and Y are not hydrogen

In another generic aspect of the invention, there is provided a compound of the formula (II):

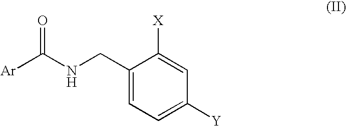

wherein for the Formula (II), the component

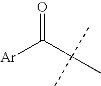

is chosen from A1-A59 in the table I below; in combination with any component

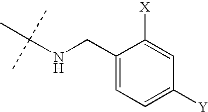

chosen from B1-B9 in the table I below;

TABLE I

| | 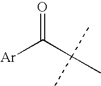 | | 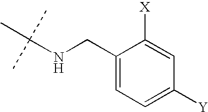 |
|---|---|---|---|
| A1 | 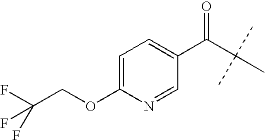 | B1 | 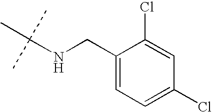 |
| A2 | 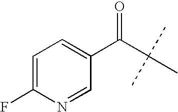 | B2 | 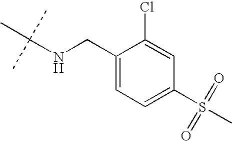 |
| A3 | 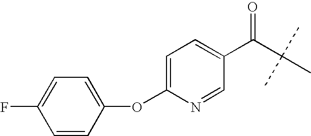 | B3 | 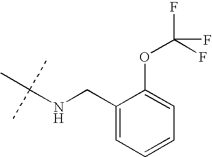 |
| A4 | 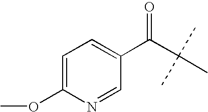 | B4 | 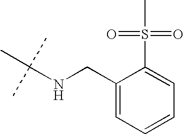 |
| A5 | 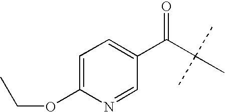 | B5 | 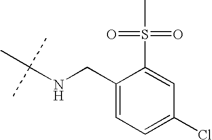 |
| A6 | 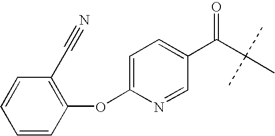 | B6 | 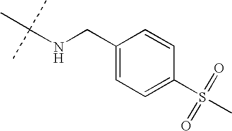 |
| A7 | 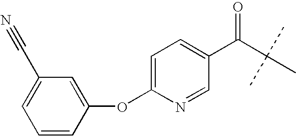 | B7 | 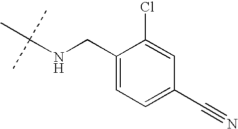 |
| A8 | 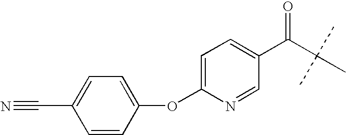 | B8 | 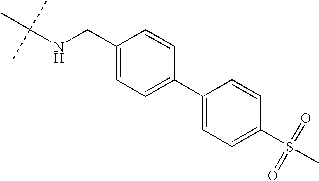 |
| A9 | 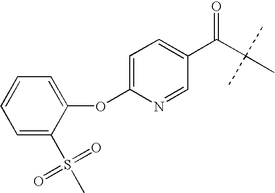 | B9 | 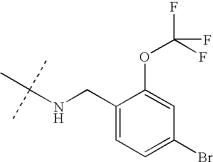 |
| A10 | 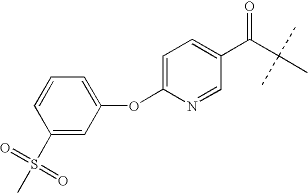 | | |
| A11 | 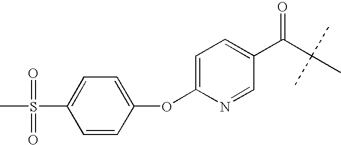 | | |
| A12 | 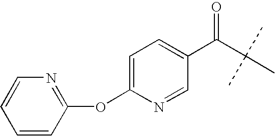 | | |
| A13 | 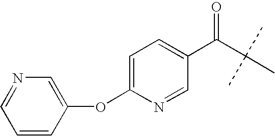 | | |
| A14 | 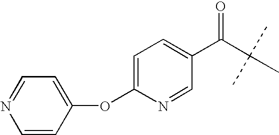 | | |
| A15 | 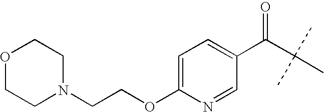 | | |
| A16 | 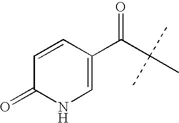 | | |
| A17 | 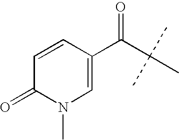 | | |
| A18 | 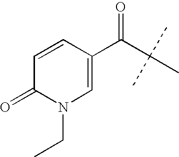 | | |
| A19 | 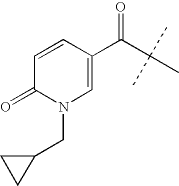 | | |
| A20 | 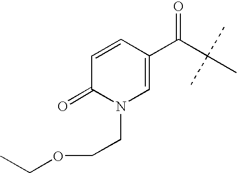 | | |
| A21 | 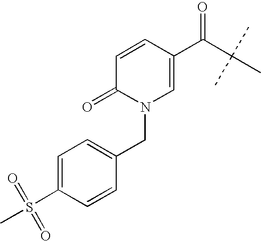 | | |
| A22 | 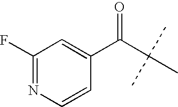 | | |
| A23 | 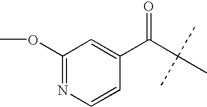 | | |
| A24 | 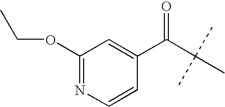 | | |
| A25 | 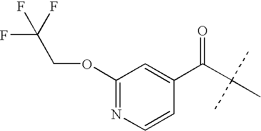 | | |
| A26 | 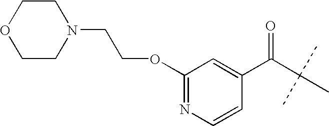 | | |
| A27 | 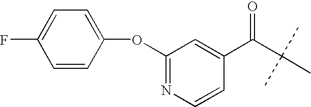 | | |
| A28 | 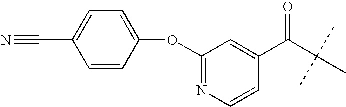 | | |
| A29 | 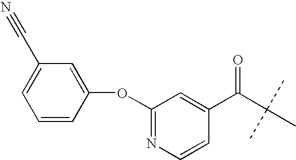 | | |
| A30 | 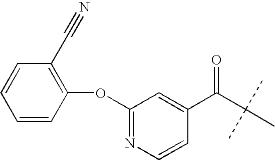 | | |
| A31 | 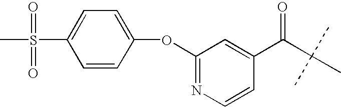 | | |
| A32 | 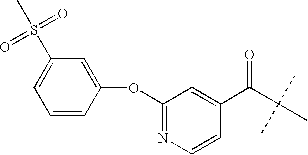 | | |
| A33 | 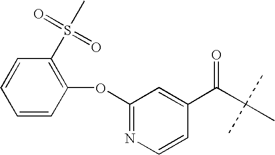 | | |
| A34 | 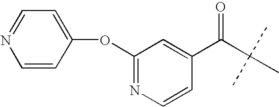 | | |
| A35 | 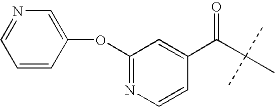 | | |
| A36 | 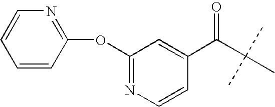 | | |
| A37 | 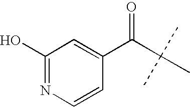 | | |
| A38 | 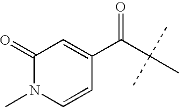 | | |
| A39 | 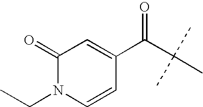 | | |
| A40 | 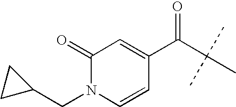 | | |
| A41 | 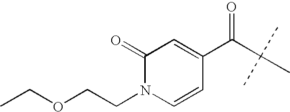 | | |
| A42 | 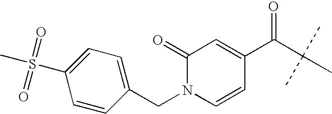 | | |
| A43 | 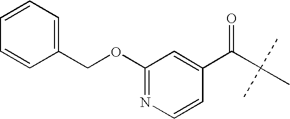 | | |
| A44 | 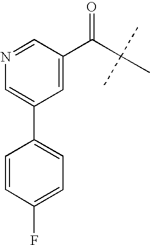 | | |
| A45 | 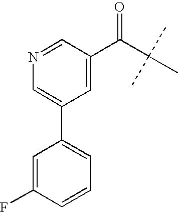 | | |
| A46 | 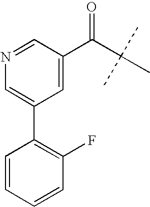 | | |
| A47 | 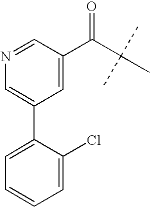 | | |
| A48 | 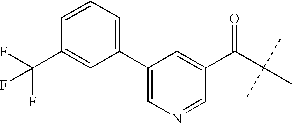 | | |
| A49 | 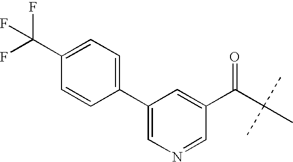 | | |
| A50 | 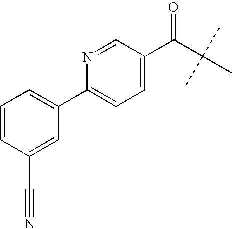 | | |
| A51 | 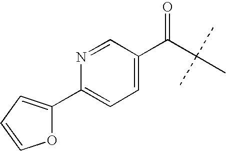 | | |
| A52 | 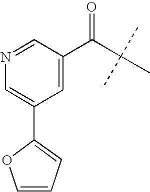 | | |
| A53 | 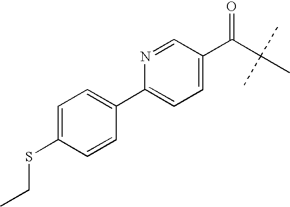 | | |
| A54 | 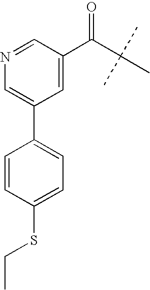 | | |
| A55 | 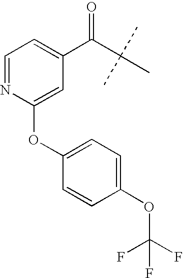 | | |
| A56 | 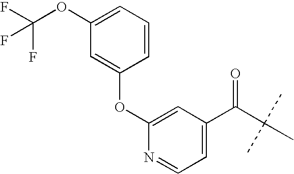 | | |
| A57 | 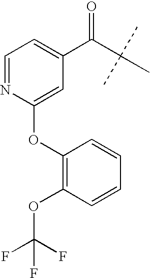 | | |
| A58 | 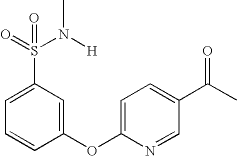 | | |
| A59 | 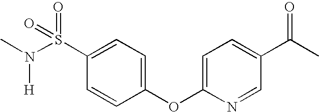 | | |

or the pharmaceutically acceptable salts thereof.

In another embodiment of the invention there is provided the following compounds, in table II, which can be made according to the general synthetic procedures and examples which follow:

TABLE II

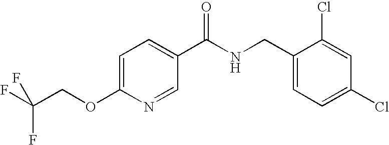

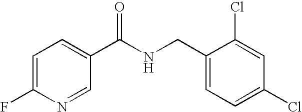

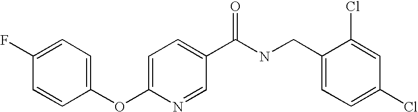

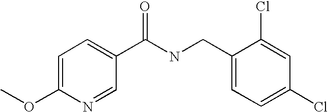

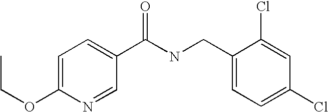

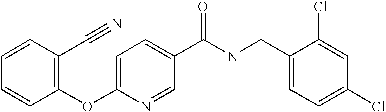

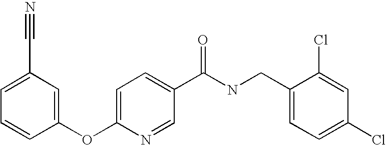

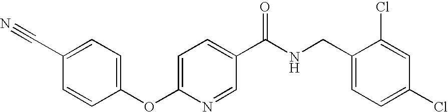

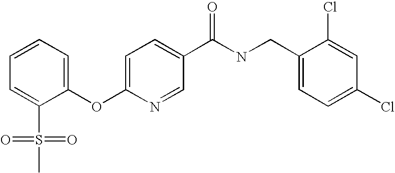

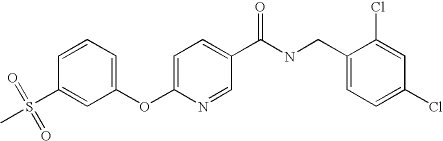

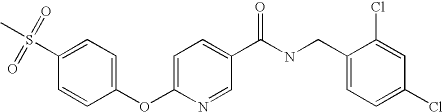

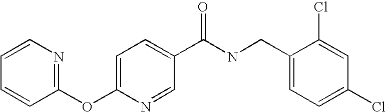

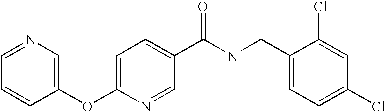

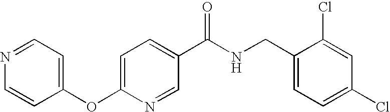

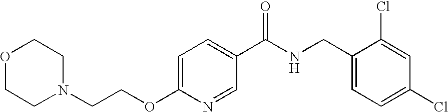

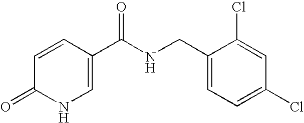

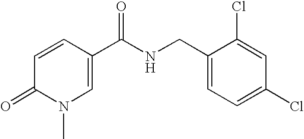

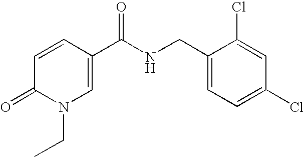

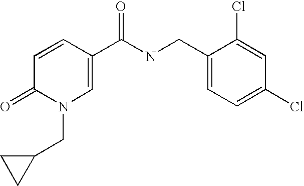

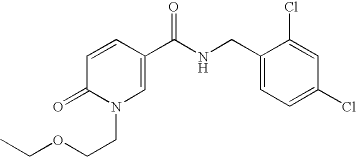

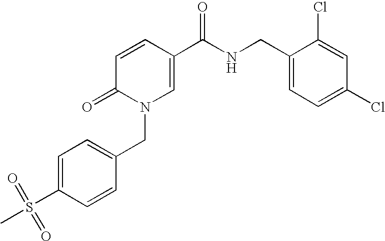

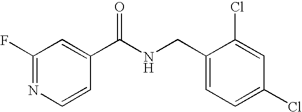

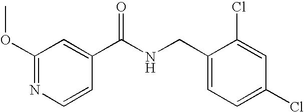

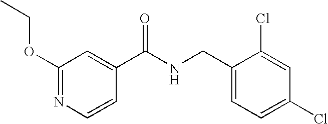

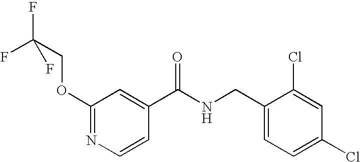

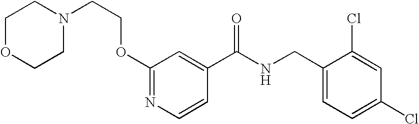

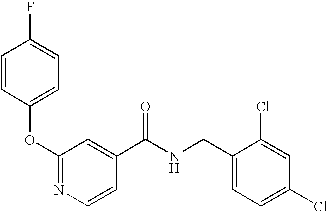

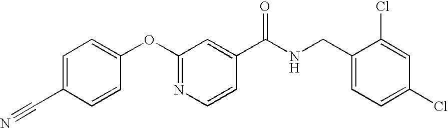

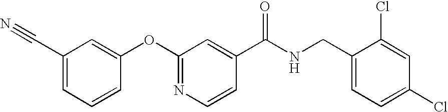

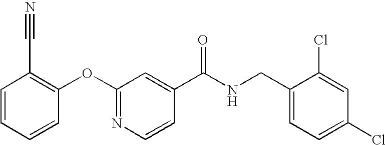

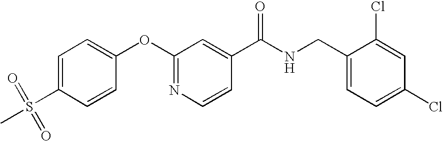

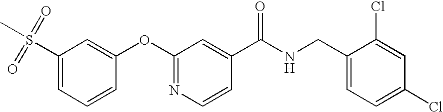

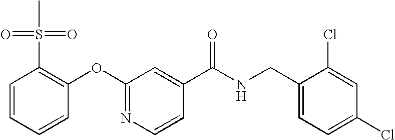

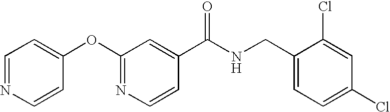

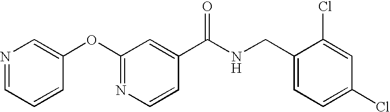

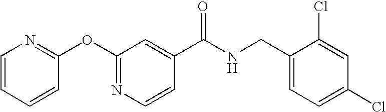

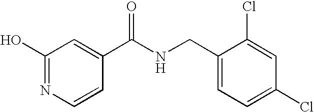

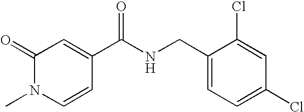

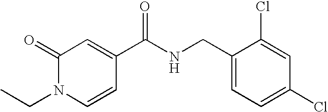

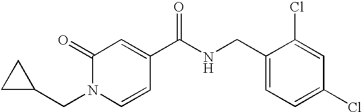

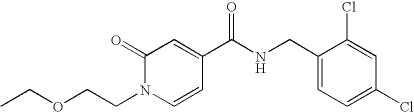

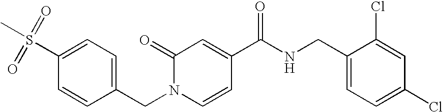

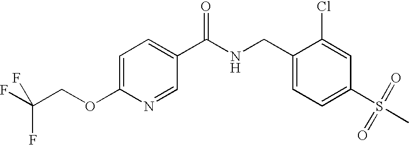

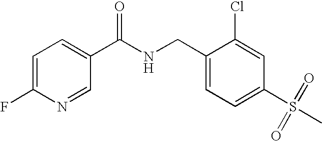

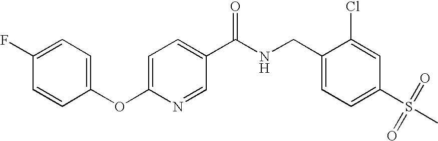

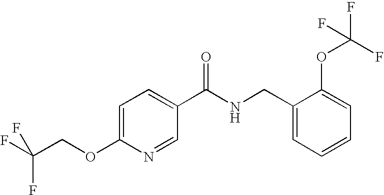

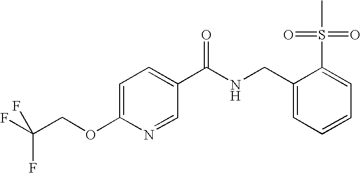

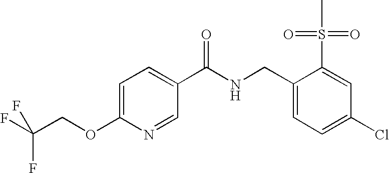

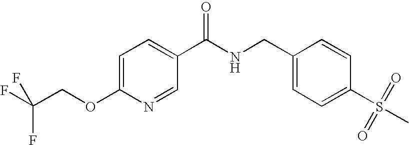

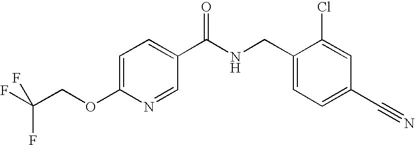

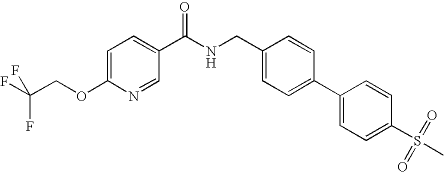

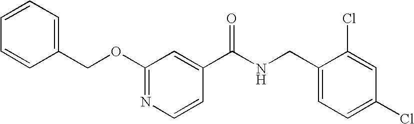

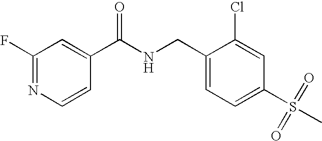

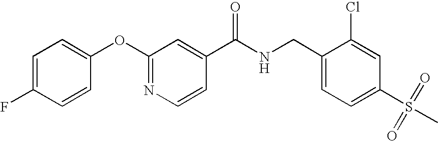

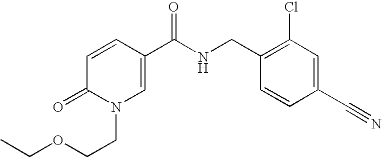

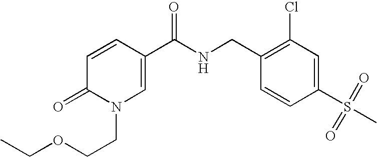

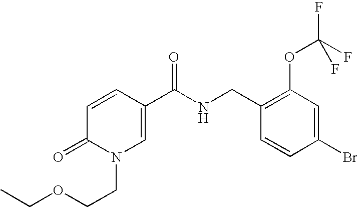

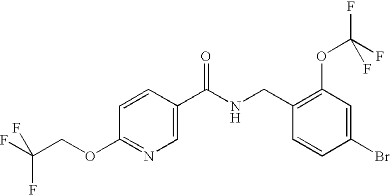

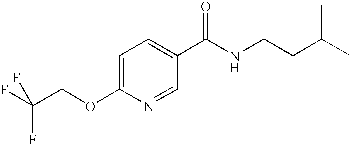

or the pharmaceutically acceptable salts thereof.

In another embodiment of the invention there is provided the following compounds, in table III, which can be made according to the general high throughput synthetic procedure which follows:

TABLE III

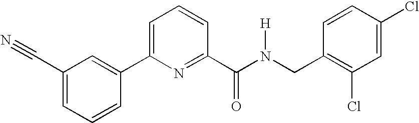

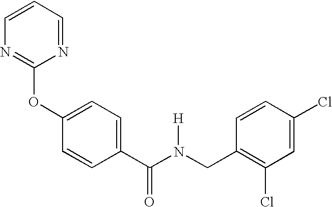

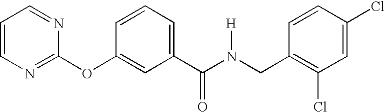

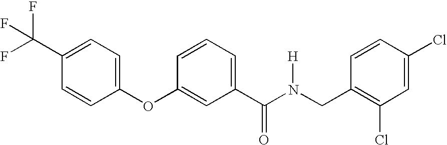

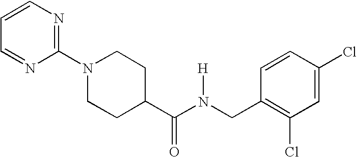

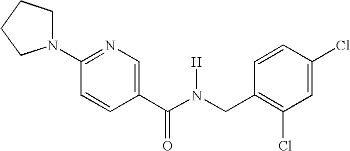

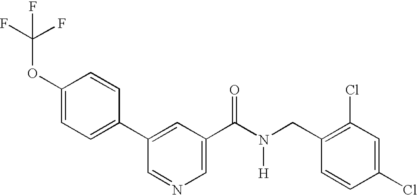

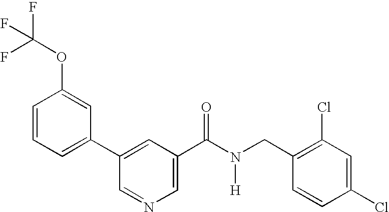

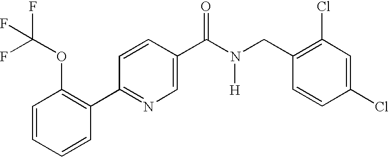

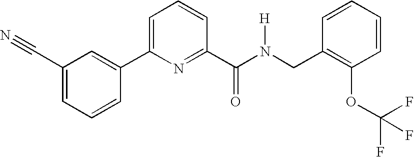

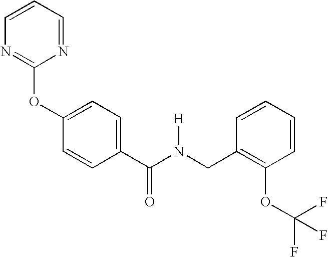

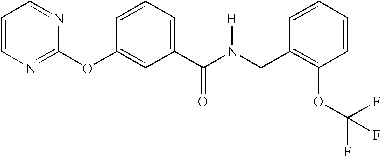

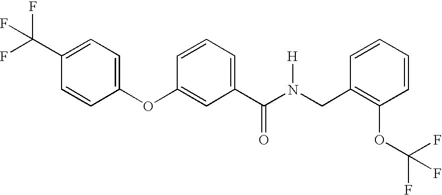

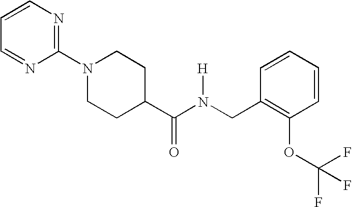

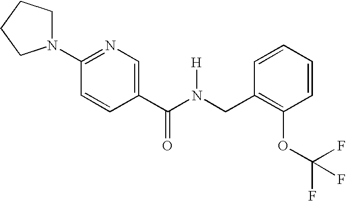

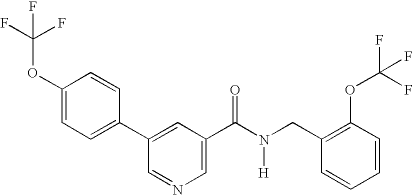

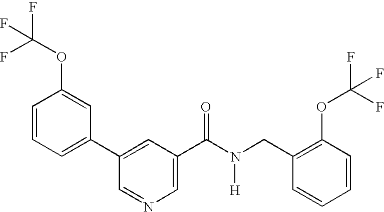

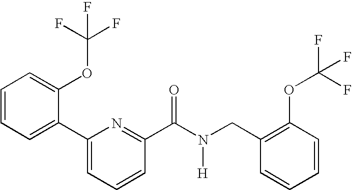

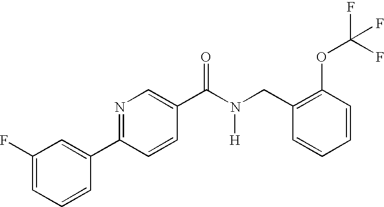

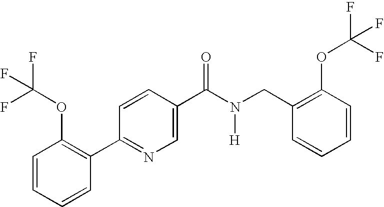

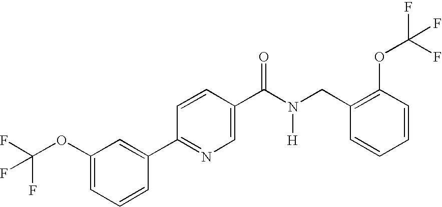

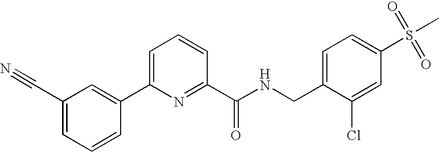

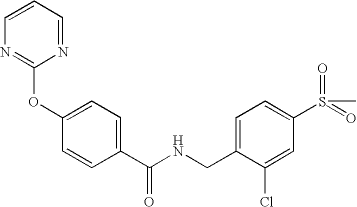

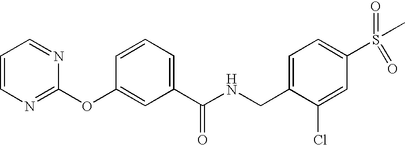

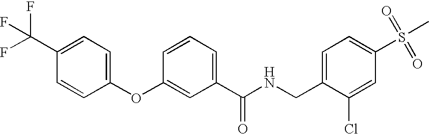

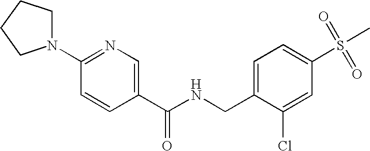

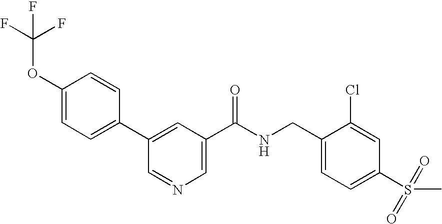

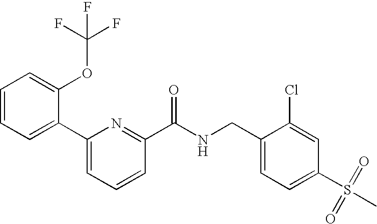

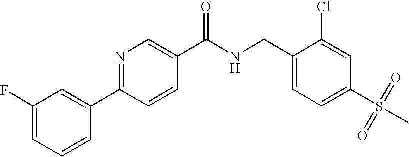

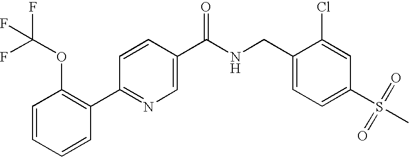

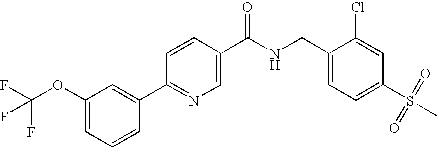

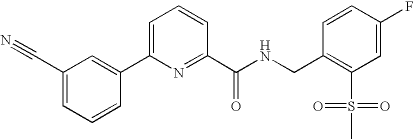

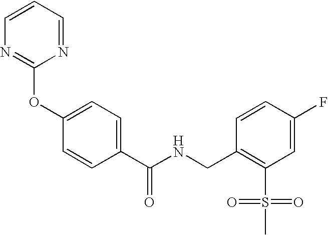

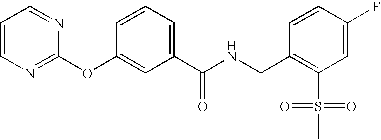

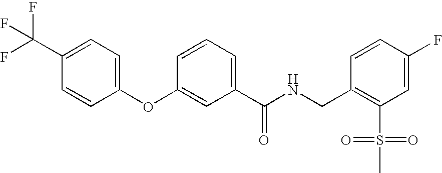

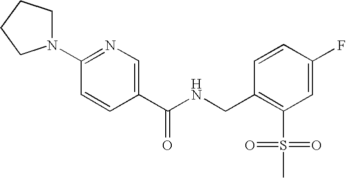

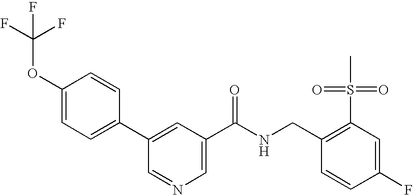

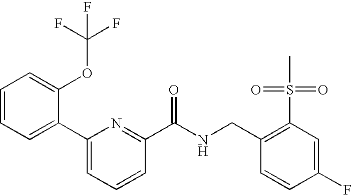

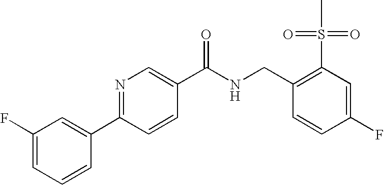

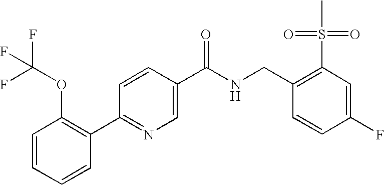

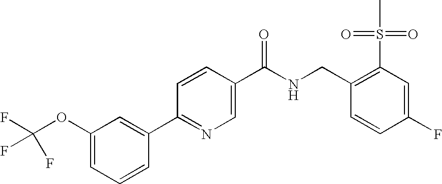

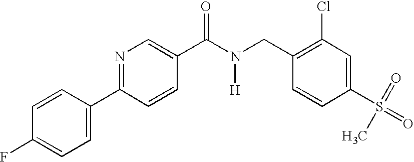

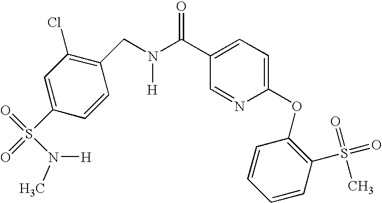

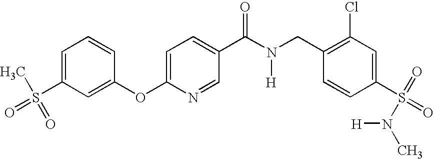

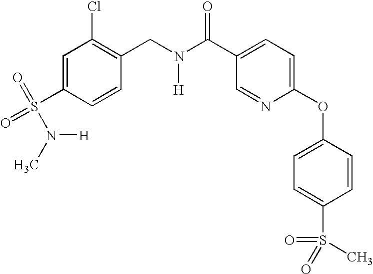

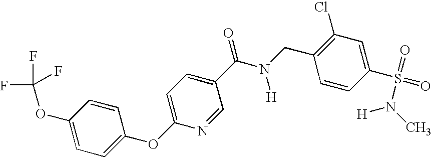

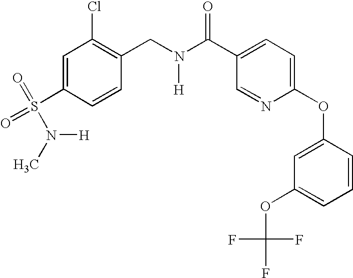

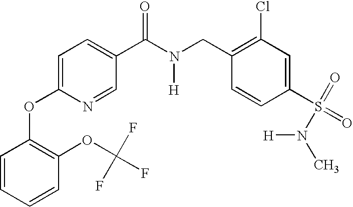

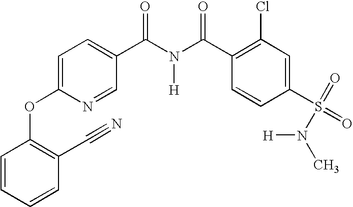

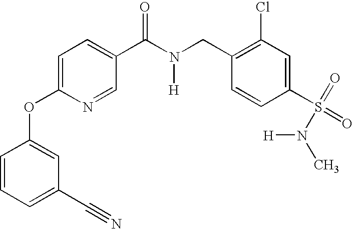

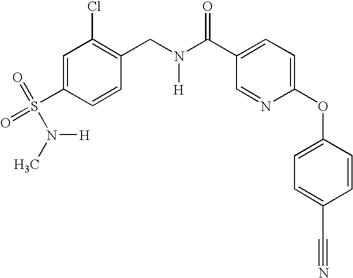

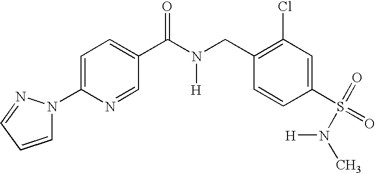

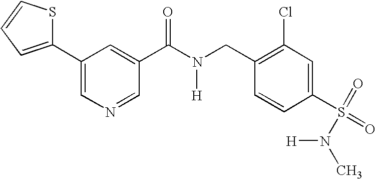

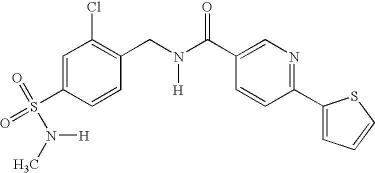

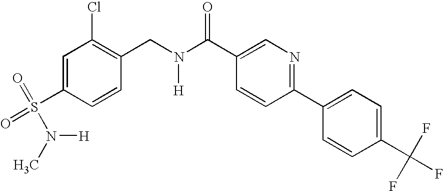

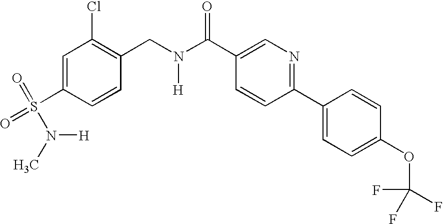

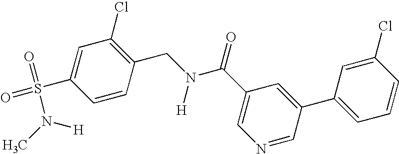

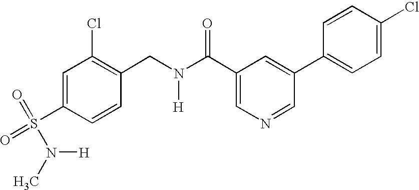

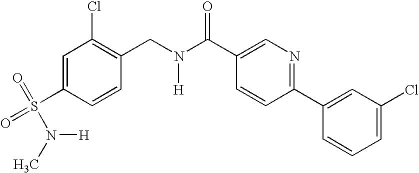

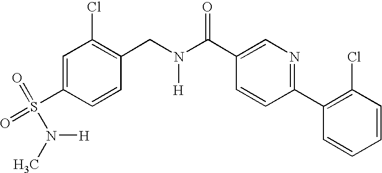

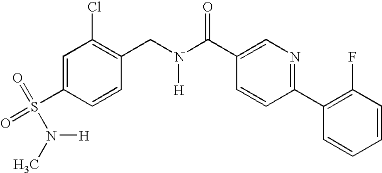

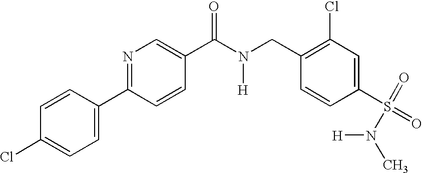

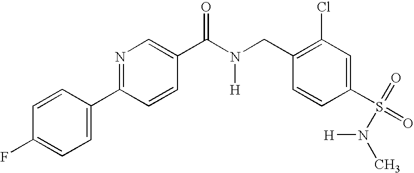

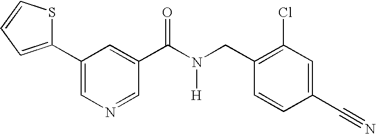

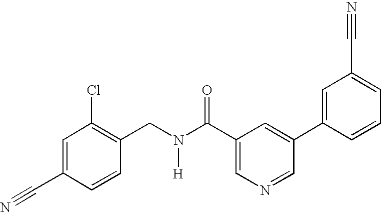

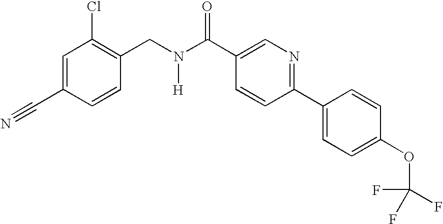

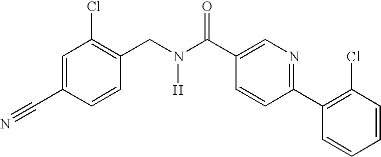

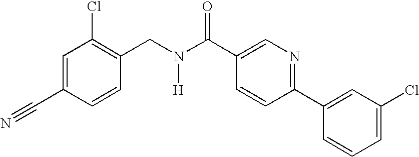

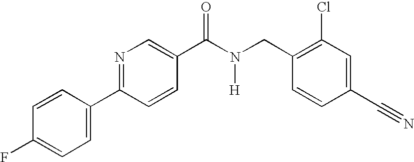

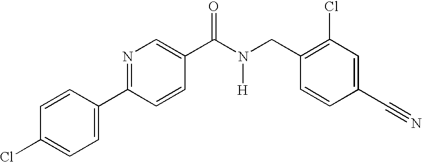

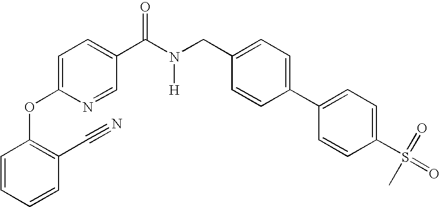

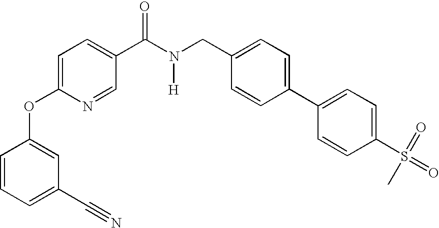

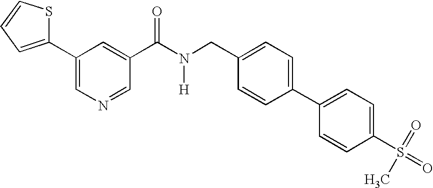

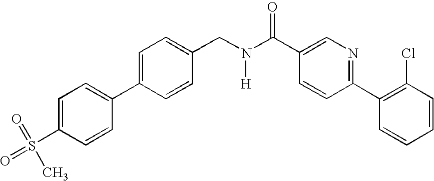

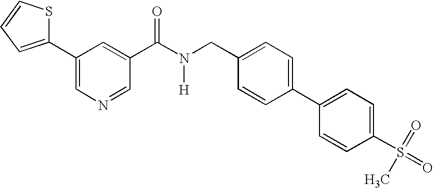

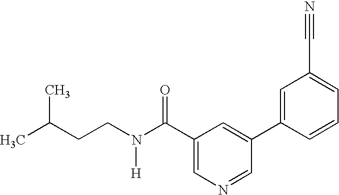

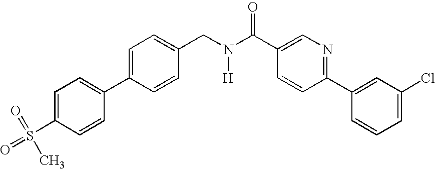

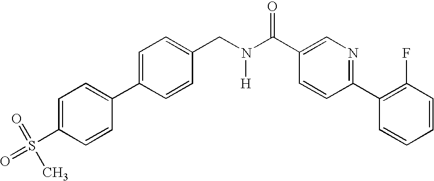

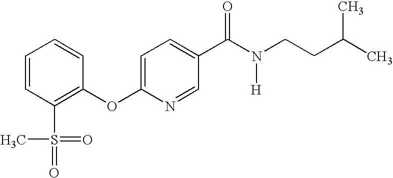

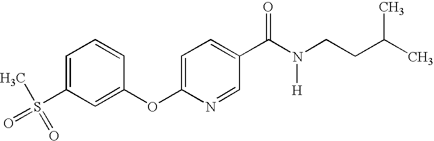

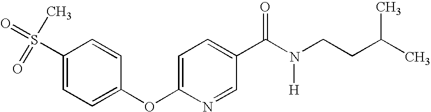

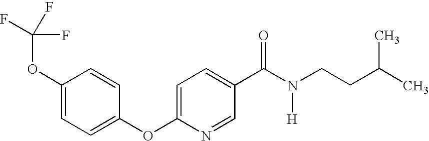

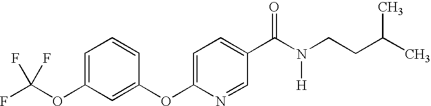

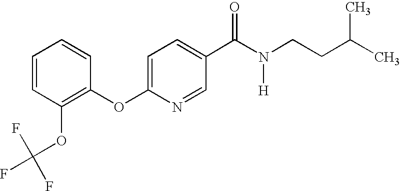

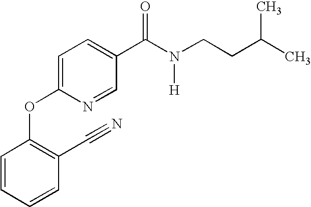

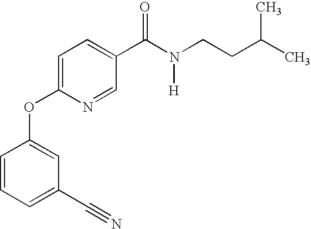

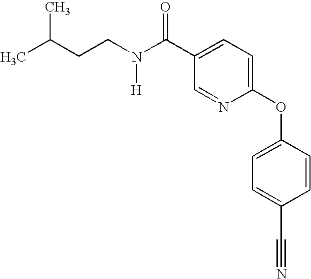

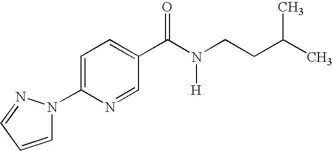

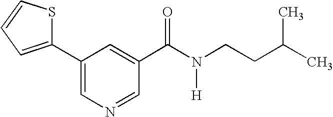

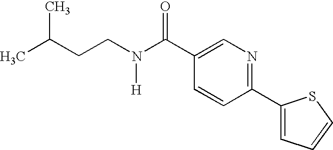

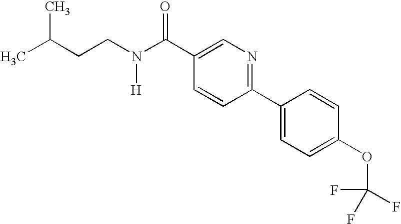

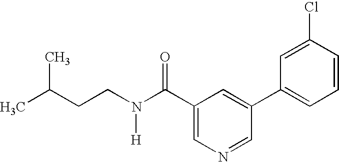

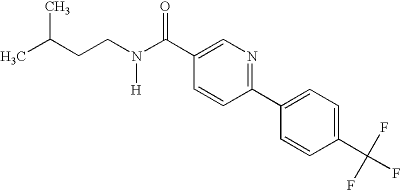

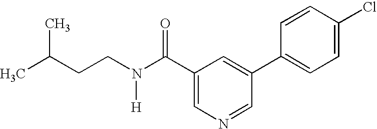

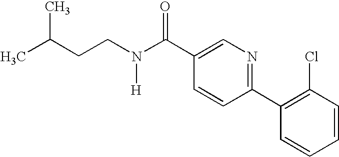

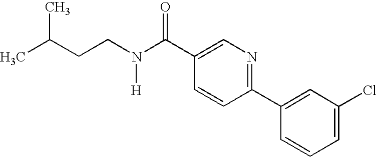

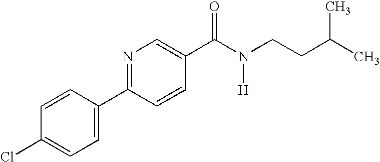

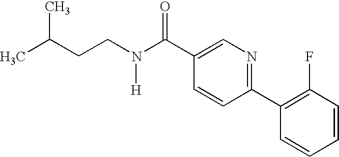

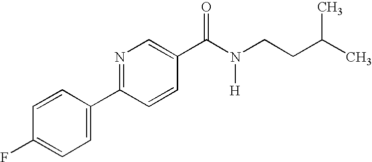

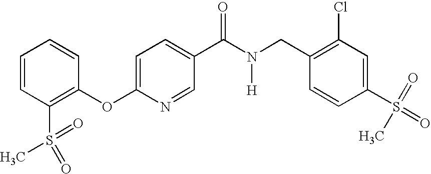

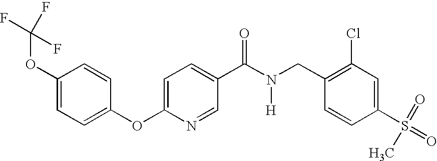

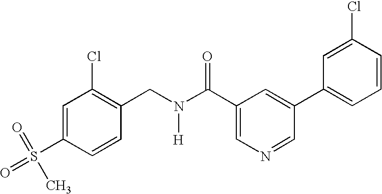

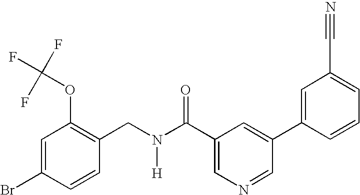

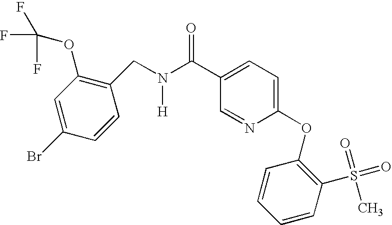

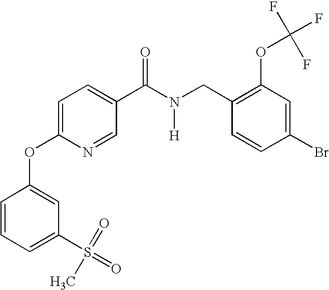

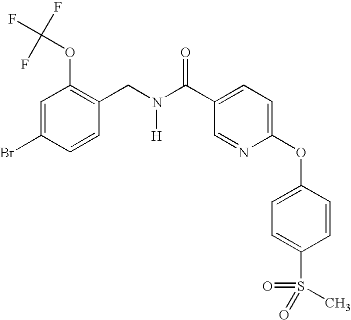

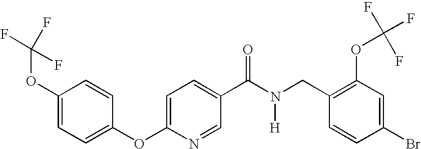

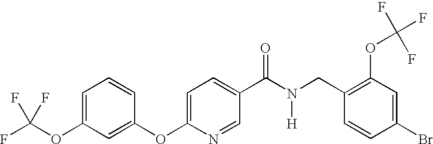

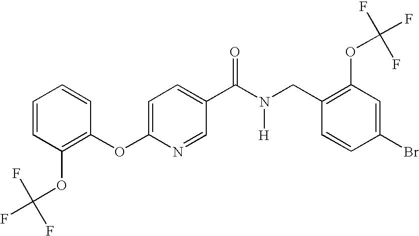

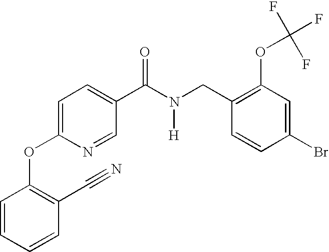

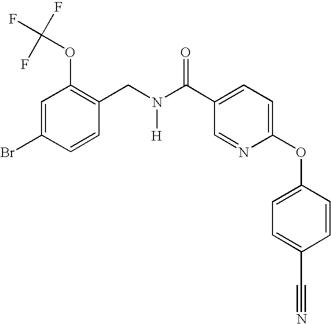

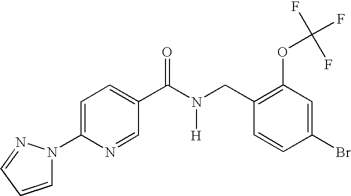

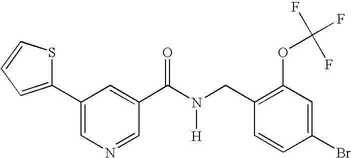

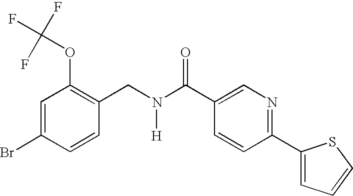

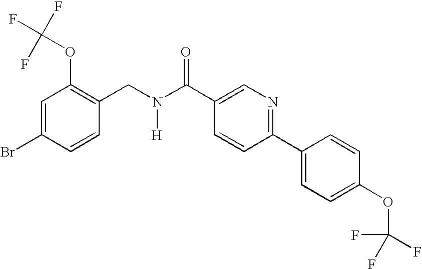

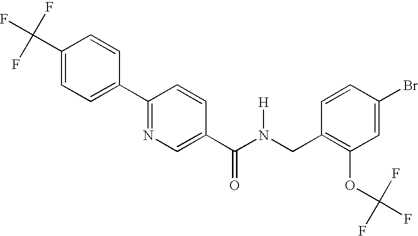

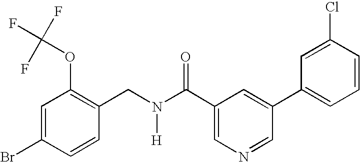

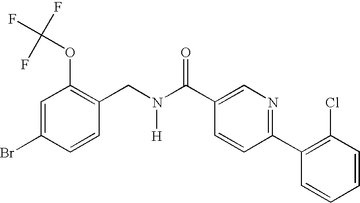

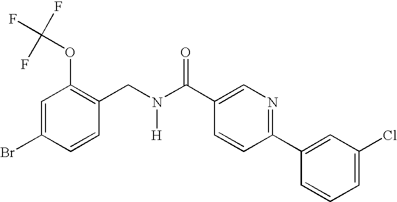

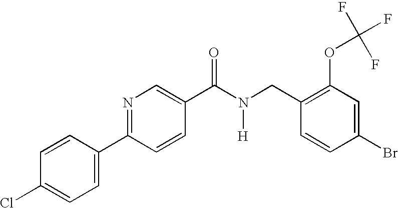

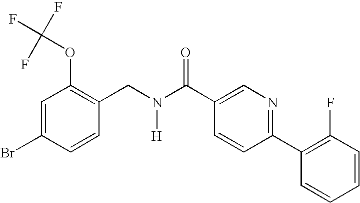

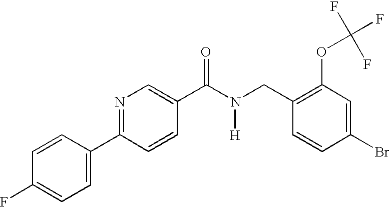

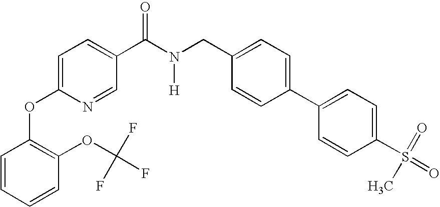

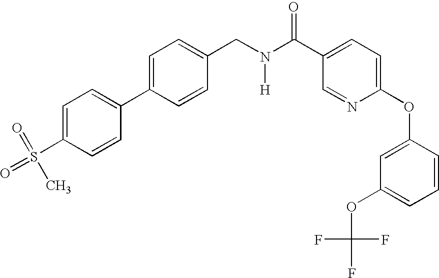

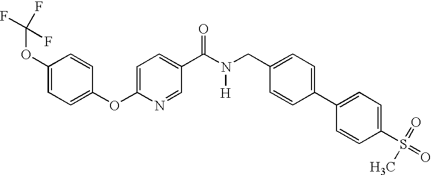

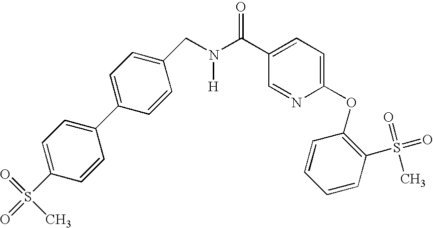

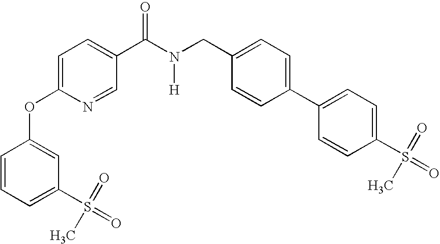

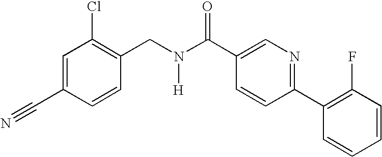

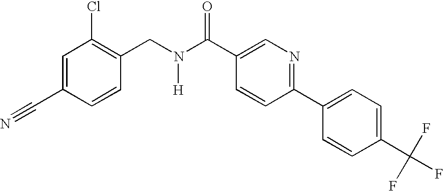

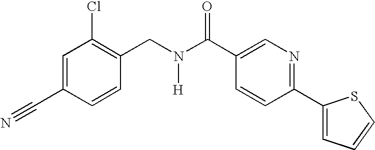

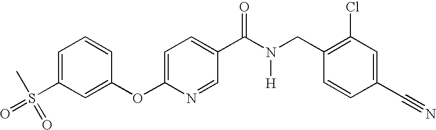

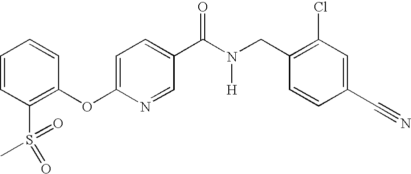

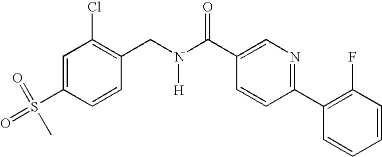

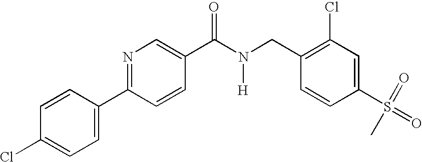

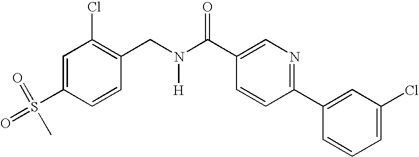

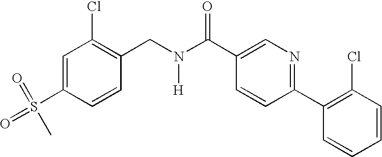

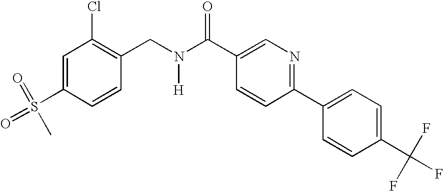

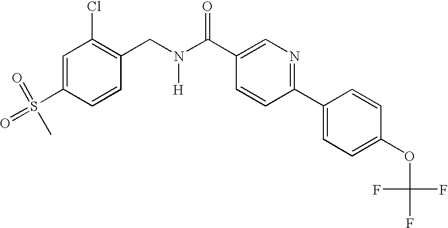

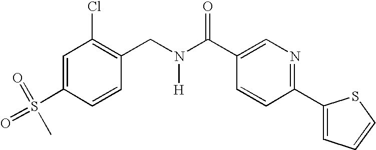

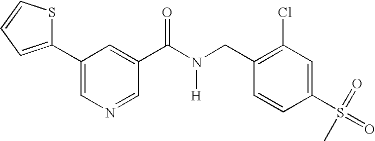

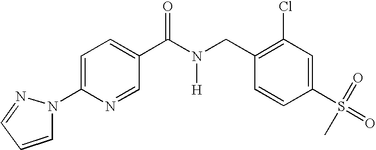

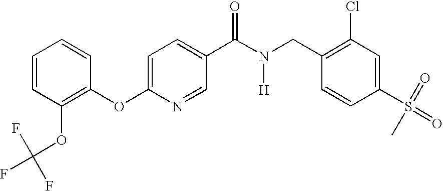

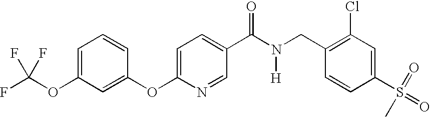

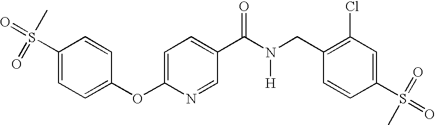

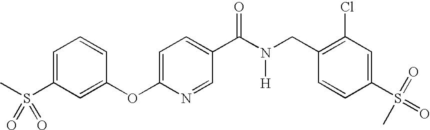

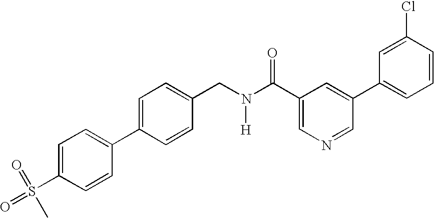

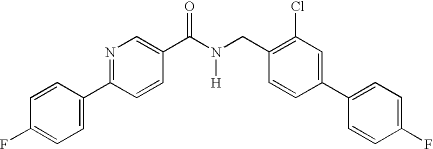

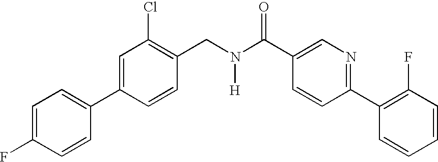

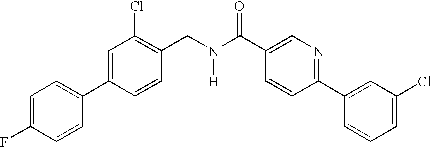

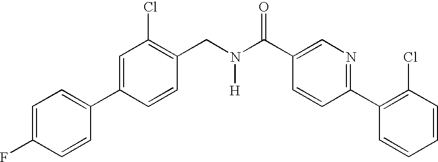

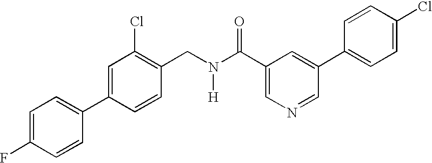

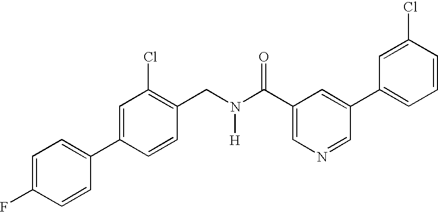

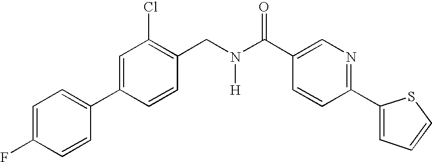

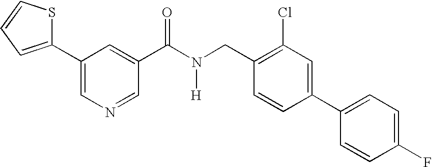

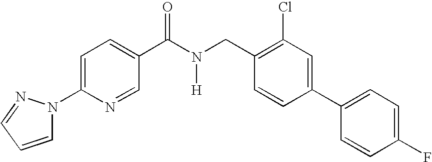

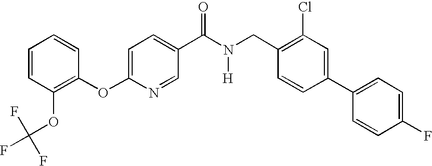

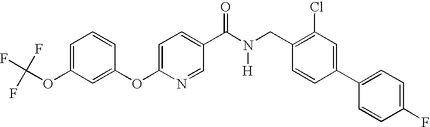

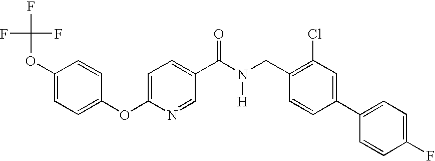

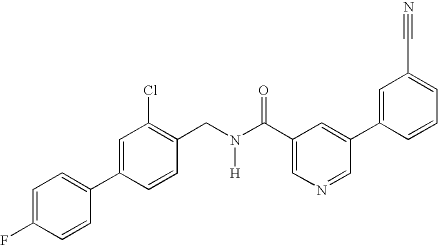

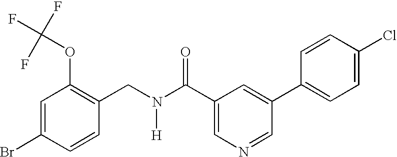

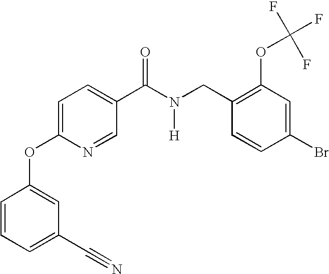

In another generic aspect of the invention, there is provided a compound of the formula (III):

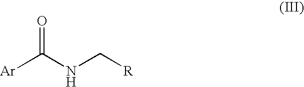

wherein for the Formula (III), the component

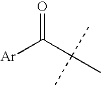

is chosen from A1-A49 in the table IV below; in combination with component B1

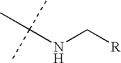

as shown in the table IV below;

TABLE IV

| | 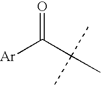 | | 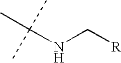 |
|---|---|---|---|
| A1 | 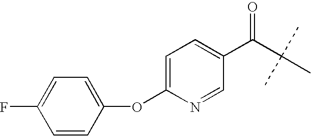 | B1 | 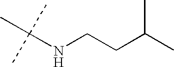 |
| A2 | 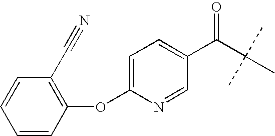 | | |
| A3 | 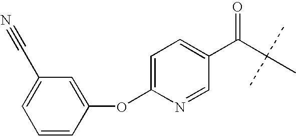 | | |
| A4 | 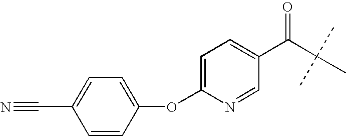 | | |
| A5 | 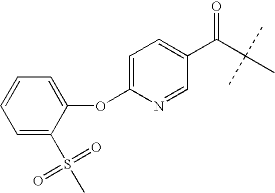 | | |
| A6 | 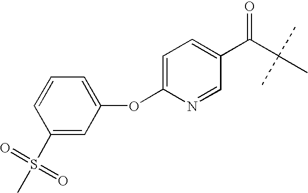 | | |
| A7 | 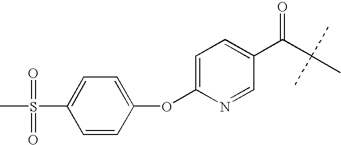 | | |
| A8 | 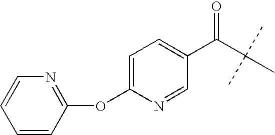 | | |
| A9 | 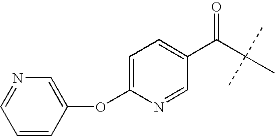 | | |
| A10 | 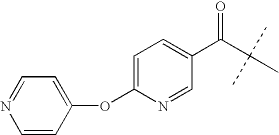 | | |
| A11 | 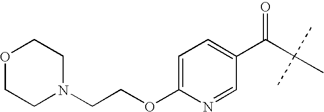 | | |
| A12 | 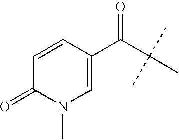 | | |
| A13 | 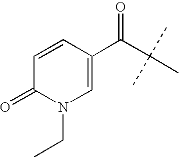 | | |
| A14 | 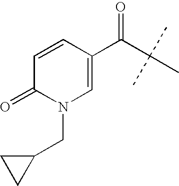 | | |
| A15 | 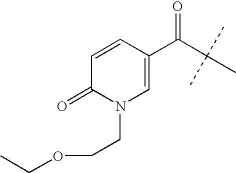 | | |
| A16 | 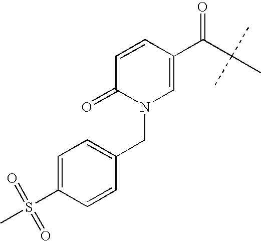 | | |
| A17 | 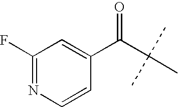 | | |
| A18 | 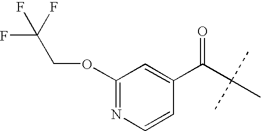 | | |
| A19 | 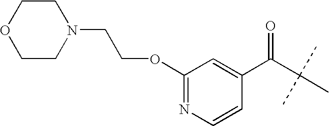 | | |
| A20 | 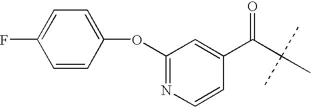 | | |
| A21 | 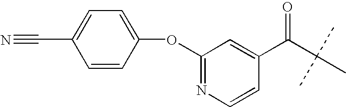 | | |
| A22 | 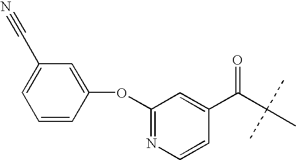 | | |
| A23 | 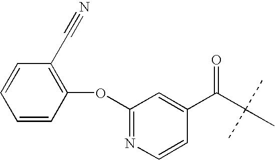 | | |
| A24 | 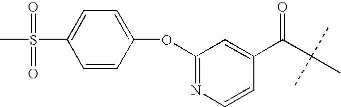 | | |
| A25 | 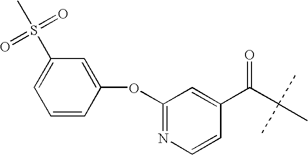 | | |
| A26 | 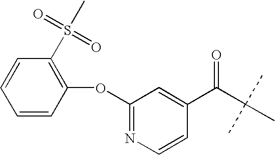 | | |
| A27 | 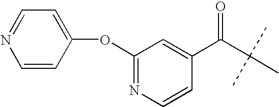 | | |
| A28 | 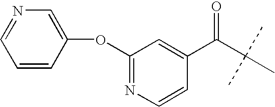 | | |
| A29 | 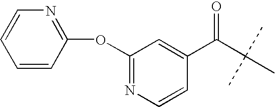 | | |
| A30 | 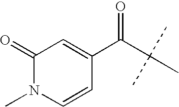 | | |
| A31 | 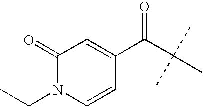 | | |
| A32 | 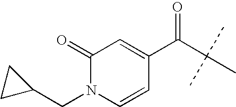 | | |
| A33 | 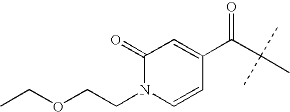 | | |
| A34 | 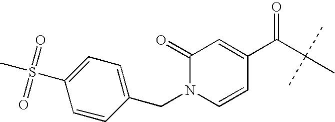 | | |
| A35 | 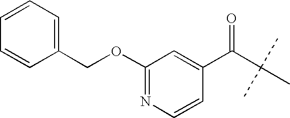 | | |
| A36 | 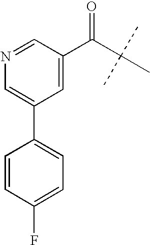 | | |
| A37 | 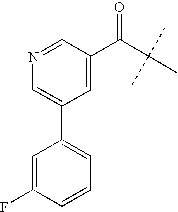 | | |
| A38 | 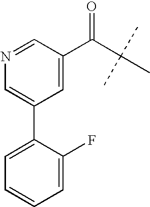 | | |
| A39 | 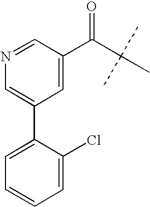 | | |
| A40 | 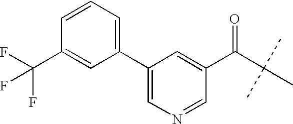 | | |
| A41 | 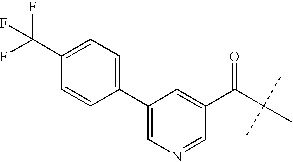 | | |
| A42 | 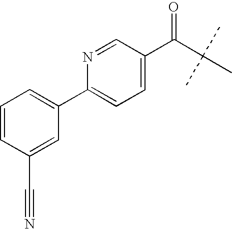 | | |
| A43 | 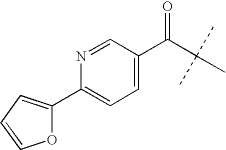 | | |
| A44 | 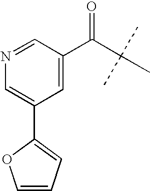 | | |
| A45 | 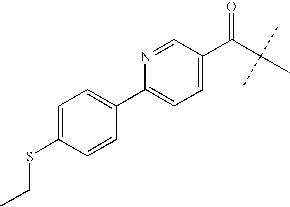 | | |
| A46 | 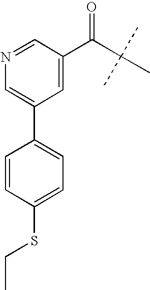 | | |
| A47 | 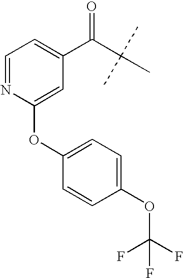 | | |
| A48 | 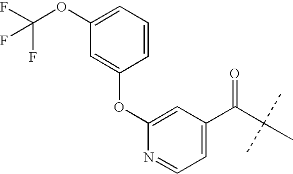 | | |
| A49 | 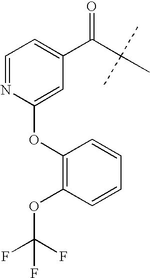 | | |

or the pharmaceutically acceptable salts thereof.

In all the compounds disclosed hereinabove in this application, in the event the nomenclature is in conflict with the structure, it shall be understood that the compound is defined by the structure.

The invention includes the use of any compounds of described above containing one or more asymmetric carbon atoms may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Some of the compounds of formulas (I, II, III) can exist in more than one tautomeric form. The invention includes methods using all such tautomers.

All terms as used herein in this specification, unless otherwise stated, shall be understood in their ordinary meaning as known in the art. For example, C₁₋₄alkoxy includes the organic radical C₁₋₄alkyl with a terminal oxygen, such as methoxy, ethoxy, propoxy, butoxy.

All organic radicals: alkyl, alkenyl and alkynyl groups, or such groups which are incorporated in other radicals such as acyl and alkoxy, shall be understood as being branched or unbranched where structurally possible and unless otherwise specified, and may be partially or fully halogenated.

The term “lower” referred to above and hereinafter in connection with organic radicals or compounds respectively defines such as branched or unbranched with up to and including 7, preferably up to and including 4 and advantageously one or two carbon atoms.

A cyclic group shall be understood to mean carbocycle, heterocycle or heteroaryl, each may be partially or fully halogenated.

An acyl group is a radical defined as —C(O)—R, where R is an organic radical or a cyclic group. Acyl represents, for example, carbocyclic or heterocyclic aroyl, cycloalkylcarbonyl, (oxa or thia)-cycloalkylcarbonyl, lower alkanoyl, (lower alkoxy, hydroxy or acyloxy)-lower alkanoyl, (mono- or di-carbocyclic or heterocyclic)-(lower alkanoyl or lower alkoxy-, hydroxy- or acyloxy-substituted lower alkanoyl), or biaroyl.

Carbocycles include hydrocarbon rings containing from three to fourteen carbon atoms. These carbocycles may be either aromatic either aromatic or non-aromatic ring systems. The non-aromatic ring systems may be mono- or polyunsaturated, monocyclic, bicyclic or tricyclic and may be bridged. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, benzyl, indanyl, indenyl, benzocyclobutanyl, dihydronaphthyl, tetrahydronaphthyl, naphthyl, decahydronaphthyl, benzocycloheptanyl, adamantyl, norbornyl, fluorene, and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms chosen from nitrogen, oxygen and sulfur. The heterocycle may be attached by any atom of the cycle, which results in the creation of a stable structure. Unless otherwise stated, heterocycles include but are not limited to, for example pyrrolidinyl, pyrrolinyl, morpholinyl, thiomorpholinyl, thiomorpholinyl sulfoxide, thiomorpholinyl sulfone, dioxalanyl, piperidinyl, piperazinyl, tetrahydrofuranyl, tetrahydropyranyl, tetrahydrofuranyl, 1,3-dioxolanone, 1,3-dioxanone, 1,4-dioxanyl, piperidinonyl, tetrahydropyrimidonyl, pentamethylene sulfide, pentamethylene sulfoxide, pentamethylene sulfone, tetramethylene sulfide, tetramethylene sulfoxide and tetramethylene sulfone.

The term “heteroaryl” shall be understood to mean an aromatic 5-8 membered monocyclic or 8-11 membered bicyclic ring containing 1-4 heteroatoms such as N,O and S. Unless otherwise stated, such heteroaryls include aziridinyl, thienyl, furanyl, isoxazolyl, oxazolyl, thiazolyl, thiadiazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl, naphthyridinyl, indazolyl, triazolyl, pyrazolo[3,4-b]pyrimidinyl, purinyl, pyrrolo[2,3-b]pyridinyl, pyrazolo[3,4-b]pyridinyl, tubercidinyl, oxazo[4,5-b]pyridinyl,

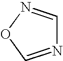

and imidazo[4,5-b]pyridinyl.

The term “heteroatom” as used herein shall be understood to mean atoms other than carbon such as oxygen, nitrogen, sulfur and phosphorous.

As used herein, “nitrogen” and “sulfur” include any oxidized form of nitrogen and sulfur and the quaternized form of any basic nitrogen. All heteroatoms in open chain or cyclic radicals include all oxidized forms.

In all alkyl groups or carbon chains one or more carbon atoms can be optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain. Such groups can be substituted as herein above described by groups such as oxo to result in definitions such as but not limited to: alkoxycarbonyl, acyl, amido and thioxo.

The term “aryl” as used herein shall be understood to mean aromatic carbocycle or heteroaryl as defined herein. Each aryl or heteroaryl unless otherwise specified includes it's partially or fully hydrogenated derivative and/or is partially or fully halogenated. For example, quinolinyl may include decahydroquinolinyl and tetrahydroquinolinyl, naphthyl may include it's hydrogenated derivatives such as tetrahydranaphthyl. Other partially or fully hydrogenated derivatives of the aryl and heteroaryl compounds described herein will be apparent to one of ordinary skill in the art.

The term “halogen” as used in the present specification shall be understood to mean bromine, chlorine, fluorine or iodine, preferably fluorine. The definitions “partially or fully halogenated”; partially or fully fluorinated; “substituted by one or more halogen atoms”, includes for example, mono, di or tri halo derivatives on one or more carbon atoms. For alkyl, a nonlimiting example would be —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The invention includes pharmaceutically acceptable derivatives of compounds of formulas (I, II, III). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the formulas (I, II, III).

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methane sulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the formulas (I, II, III). Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed herein above, thereby imparting the desired pharmacological effect.

The compounds described herein are either commercially available or can be made by methods and any necessary intermediates well known in the art.

In order that this invention be more fully understood, the following examples are set forth. These examples are for the purpose of illustrating preferred embodiments of this invention, and are not to be construed as limiting the scope of the invention in any way.

The examples which follow are illustrative and, as recognized by one skilled in the art, particular reagents or conditions could be modified as needed for individual compounds without undue experimentation. Starting materials used in the scheme below are either commercially available or easily prepared from commercially available materials by those skilled in the art.

### General Synthetic Methods

The invention also provides processes for making compounds of Formula (I) and (II). In all schemes, unless specified otherwise, Ar, X and Y in the formulas below shall have the meaning of Ar, X and Y in Formula (I) and (II) of the invention described herein above.

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography (TLC), if desired, and intermediates and products may be purified by chromatography on silica gel and/or by recrystallization. The appropriately substituted starting materials and intermediates used in the preparation of compounds of the invention are either commercially available or readily prepared by methods known in the literature to those skilled in the art, and are illustrated in the synthetic examples below. Reference in this regard can be made to U.S. provisional application Nos. 60/678,828 and 60/678,871, incorporated herein be reference.

Compounds of Formula (I) and (II) may be synthesized by the method illustrated in Scheme 1

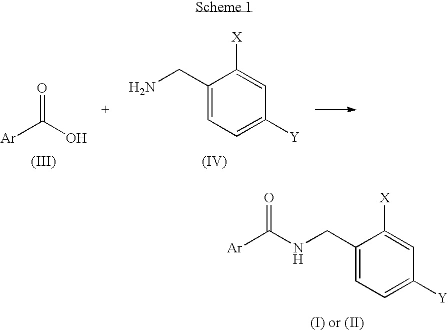

Amide coupling of the carboxylic acid (III) with the desired amine (IV) provides the desired compound of formula (I) or (II). Standard peptide coupling reactions known in the art (see for example M. Bodanszky, 1984, The Practice of Peptide Synthesis, Springer-Verlag) may be employed in these syntheses. An example of suitable coupling conditions is treatment of a solution of the carboxylic acid in a suitable solvent such as DMF with EDC, HOBT, and a base such as diisopropylethylamine, followed by the desired amine.

Alternatively, reaction of the carboxylic acid with reagents such as oxalyl chloride provides the corresponding acid chloride. Reaction of the acid chloride with the desired amine (IV) in a suitable solvent provides the compound of formula (I) or (II).

Further modification of the initial product of formula (I) or (II) by methods known in the art and illustrated in the Examples below, may be used to prepare additional compounds of this invention.

Compounds of Formula (I) and (II), wherein Ar is O-substituted pyridine, may be synthesized by the method illustrated in Scheme 2

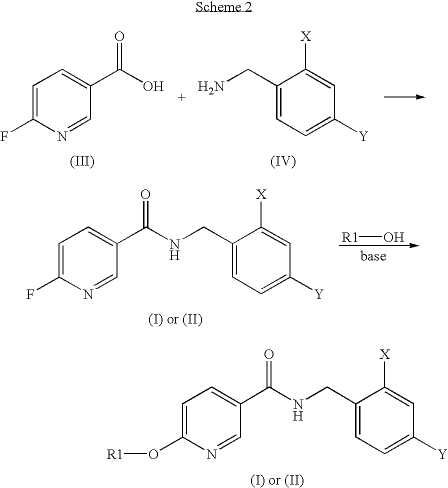

Amide coupling of the carboxylic acid (III) with the desired amine (IV) provides the desired compound of formula (I) or (II) wherein Ar is 2-fluoropyridine. Standard peptide coupling reactions known in the art (see for example M. Bodanszky, 1984, The Practice of Peptide Synthesis, Springer-Verlag) may be employed in these syntheses. Alternatively, reaction of the carboxylic acid with reagents such as oxalyl chloride provides the corresponding acid chloride. Reaction of the acid chloride with the desired amine (IV) in a suitable solvent provides the compound of formula (I) or (II). Reacting the coupled 2-fluoropyridine compound with suitable oxygen containing nucleophiles such as R1-OH, in a suitable solvent, in the presence of a suitable base provides the desired O-substituted product of formula (I) or (II)

Compounds of Formula (I) and (II), wherein Ar is N-substituted pyridinone, may be synthesized by the method illustrated in Scheme 3

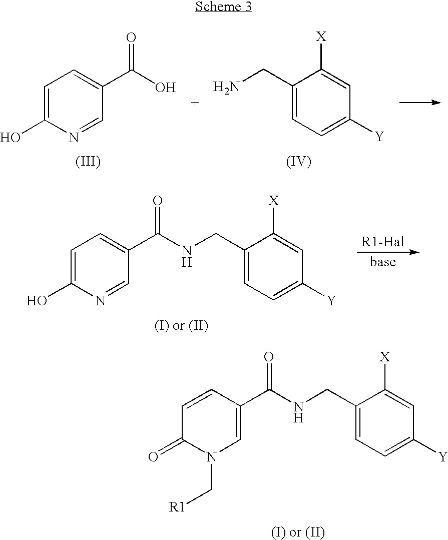

Amide coupling of the carboxylic acid (III) with the desired amine (IV) provides the desired compound of formula (I) or (II) wherein Ar is 2-hydroxypyridine. Standard peptide coupling reactions known in the art (see for example M. Bodanszky, 1984, The Practice of Peptide Synthesis, Springer-Verlag) may be employed in these syntheses. Alternatively, reaction of the carboxylic acid with reagents such as oxalyl chloride provides the corresponding acid chloride. Reaction of the acid chloride with the desired amine (IV) in a suitable solvent provides the compound of formula (I) or (II). Reacting the coupled 2-hydroxypyridine compound with suitable alkylating agents such as R1-Hal, wherein Hal is Cl, Br, or I, in a suitable solvent, in the presence of a suitable base provides the desired N-substituted pyridinone of formula (I) or (II)

The appropriately substituted starting materials and intermediates used in the preparation of compounds of the invention are either commercially available or readily prepared by methods known in the literature to those skilled in the art, and are illustrated in the synthetic examples below.

### EXAMPLE 1

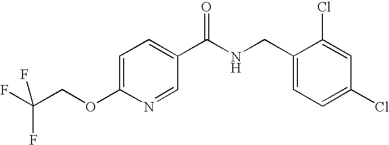

### N-(2,4-Dichloro-benzyl)-6-(2,2,2-trifluoro-ethoxy)-nicotinamide

To a solution of 6-(2,2,2-Trifluoroethoxy)nicotinic acid (0.224 g, 1.01 mmol) in N,N,dimethylformamide (5 mL) is added 2,4 dichlorobenzylamine (0.15 mL, 1.01 mmol) followed by the addition of 1-hydroxybenzotriazole (0.279 g, 2.06 mmol), 1-ethyl-3-(3-dimethylaminopropyl)-carbodiimide hydrochloride (0.420 g, 2.20 mmol) and diisopropylethylamine (0.72 mL, 4.40 mmol). The reaction is stirred overnight. The mixture is diluted with water and the product collected by vacuum filtration. The product is further purified by tirturation with hexanes/ether to give the title compound (0.215 g, 56%). LCMS: 380.11 (M+H⁺).

### EXAMPLE 2

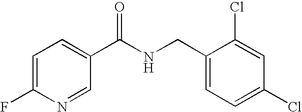

### N-(2,4-Dichloro-benzyl)-6-fluoro-nicotinamide

The compound is prepared and purified using the procedure from example 1, starting from 6-fluoronicotininc acid (2.28 g, 16.0 mmol), 2,4 dichlorobenzylamine (2.15 mL, 16.0 mmol), 1-hydroxybenzotriazole (5.0 g, 37.0 mmol), 1-ethyl-3-(3-dimethylaminopropyl)-carbodiimide hydrochloride (7.05 g, 37.0 mmol) and diisopropylethylamine (7.18 mL, 40.0 mmol) N,N,dimethylformamide (100 mL) to provide the title compound (3.9 g, 80.7%). LCMS: 300.00 (M+H⁺).

### EXAMPLE 3

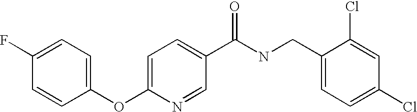

### N-(2,4-Dichloro-benzyl)-6-(4-fluoro-phenoxy)-nicotinamide

To a suspension of sodium hydride (60% in mineral oil, 50.0 mg 1.25 mmol), in N,N dimethylacetamide (3.0 mL) is added 4-fluorophenol (140.1 mg, 1.25 mmol) portiowise followed by N-(2,4-Dichloro-benzyl)-6-fluoro-nicotinamide (300 mg, 1.00 mmol). The reaction vessel is scaled and subjected to microwave irradiation at 140° C. for 45 min. The reaction is poured into ether/ethyl acetate and washed with water and brine. The organic layer is dried (MgSO₄) filtered and evaporated to dryness. The resulting solid is purified opn silica gel with dichloromethane/methanol as the eluent to provide the title compound (312.0 mg, 79.5%) LC/MS: 392.24 (M+H⁺).

### EXAMPLE 4

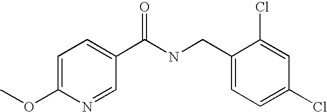

### N-(2,4-Dichloro-benzyl)-6-methoxy-nicotinamide

The compound is prepared and purified using the procedure from example 3, starting from sodium hydride (60% in mineral oil, 50.0 mg 1.25 mmol), methanol (0.078 mL, 1.75 mmol), N-(2,4-Dichloro-benzyl)-6-fluoro-nicotinamide (300 mg, 1.00 mmol), and N,N dimethylacetamide (3.0 mL) to provide the title compound (0.17 g, 54.5%). LCMS: found: 312.16 (M+H⁺).

### EXAMPLE 5

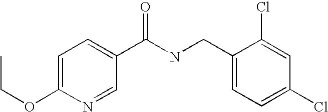

### N-(2,4-Dichloro-benzyl)-6-ethoxy-nicotinamide

The compound is prepared and purified using the procedure from example 3, starting from ethanol (0.102 mL, 1.75 mmol), sodium hydride (60% in mineral oil, 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.19 g, 60.4%). LCMS: 326.12 (M+H⁺).

### EXAMPLE 6

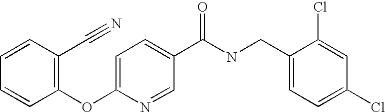

### 6-(2-Cyano-phenoxy)-N-(2,4-dichloro-benzyl)-nicotinamide

The compound is prepared and purified using the procedure from example 3, starting from 2-cyanophenol (0.148 g, 1.75 mmol), sodium hydride (60% in mineral oil, 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.10 g, 25.0%). LCMS: 399.17 (M+H⁺).

### EXAMPLE 7

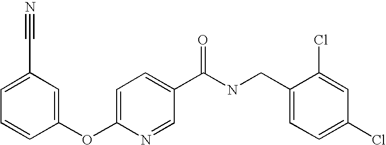

### 6-(3-Cyano-phenoxy)-N-(2,4-dichloro-benzyl)-nicotinamide

The compound is prepared and purified using the procedure from example 3, starting from 3-cyanophenol (0.148 g, 1.75 mmol), sodium hydride (60% in mineral oil, 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.10 g, 25.0%). LCMS: 399.11 (M+H⁺).

### EXAMPLE 8

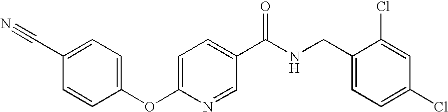

### 6-(4-Cyano-phenoxy)-N-(2,4-dichloro-benzyl)-nicotinamide

The compound is prepared and purified using the procedure from example 3, starting from 4-cyanophenol (0.148 g, 1.75 mmol), sodium hydride (60% in mineral oil, 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.10 g, 25.0%). LCMS: 399.21 (M+H⁺).

### EXAMPLE 9

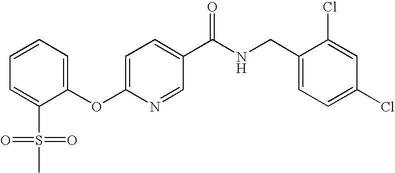

### N-(2,4-Dichloro-benzyl)-6-(2-methanesulfonyl-phenoxy)-nicotinamide

The compound is prepared and purified using the procedure from example 3, starting from 2-methanesulfonylphenol (0.215 g, 1.75 mmol), sodium hydride (60% in mineral oil, 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.10 g, 25.0%). LCMS: 452.82 (M+H⁺).

### EXAMPLE 10

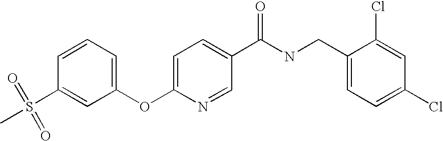

### N-(2,4-Dichloro-benzyl)-6-(3-methanesulfonyl-phenoxy)-nicotinamide

The compound is prepared and purified using the procedure from example 3, starting from 3-methanesulfonylphenol (0.215 g, 1.75 mmol, Bordwell, F. G.; et. al. J. Am. Chem. Soc. 1991, 113, 1736.), sodium hydride (60% in mineral oil, 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.10 g, 25.0%). LCMS: 452.15 (M+H⁺).

### EXAMPLE 11

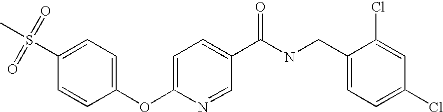

### N-(2,4-Dichloro-benzyl)-6-(4-methanesulfonyl-phenoxy)-nicotinamide

The compound is prepared and purified using the procedure from example 3, starting from 4-methanesulfonylphenol (0.215 g, 1.75 mmol), sodium hydride (60% in mineral oil, 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.10 g, 25.0%). LCMS: 452.17 (M+H⁺).

### EXAMPLE 12

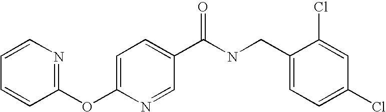

### N-(2,4-Dichloro-benzyl)-6-(pyridin-2-yloxy)-nicotinamide

The compound is prepared and purified using the procedure from example 3, starting from 2-hydroxypyridine (0.119 g, 1.75 mmol), sodium hydride (60% in mineral oil, 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.09 g, 25.6%). LCMS: 375.24 (M+H⁺).

### EXAMPLE 13

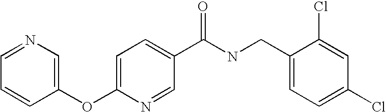

### N-(2,4-Dichloro-benzyl)-6-(pyridin-3-yloxy)-nicotinamide

The compound is prepared and purified using the procedure from example 3, starting from 3-hydroxypyridine (0.119 g, 1.75 mmol), sodium hydride (60% in mineral oil 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.151 g, 40.2%). LCMS: 375.25 (M+H⁺).

### EXAMPLE 14

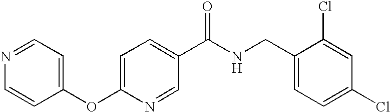

### N-(2,4-Dichloro-benzyl)-6-(pyridin-4-yloxy)-nicotinamide

The compound is prepared and purified using the procedure from example 3, starting from 4-hydroxypyridine (0.119 g, 1.75 mmol), sodium hydride (60% in mineral oil, 50.0 mg 1.25 mmol), N-(2,4-Dichloro-benzyl)-6-fluoro-nicotinamide (300 mg, 1.00 mmol), and N,N dimethylacetamide (3.0 mL), to provide the title compound (0.235 g, 62.6%). LCMS: 375.07 (M+H⁺).

### EXAMPLE 15

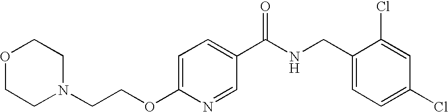

### N-(2,4-Dichloro-benzyl)-6-(2-morpholin-4-yl-ethoxy)-nicotinamide

The compound is prepared and purified using the procedure from example 3, starting from 2-morpholin-4-yl ethanol (0.182 mL, 1.50 mmol), sodium hydride (60% in mineral oil, 50.0 mg 1.25 mmol), N-(2,4-Dichloro-benzyl)-6 fluoro-nicotinamide (300 mg, 1.00 mmol), and N,N dimethylacetamide (3.0 mL), to provide the title compound (0.200 g, 48.6%). LCMS: 411.29 (M+H⁺).

### EXAMPLE 16

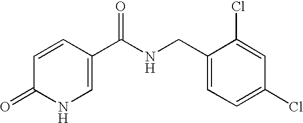

### N-(2,4-Dichloro-benzyl)-6-hydroxy-nicotinamide

The compound is prepared and purified using the procedure from example 1, starting from 6-hydroxynicotininc acid (0.42 g, 3.0 mmol), 2,4 dichlorobenzylamine (0.40 mL, 3.0 mmol), 1-hydroxybenzotriazole (0.81 g, 6.0 mmol), 1-ethyl-3-(3-dimethylaminopropyl)-carbodiimide hydrochloride (1.14 g, 6.0 mmol) and diisopropylethylamine (1.46 mL, 9.0 mmol) N,N,dimethylformamide (190 mL) to provide the title compound (0.69 g, 78%). LCMS: 298.14 (M+H⁺).

### EXAMPLE 17

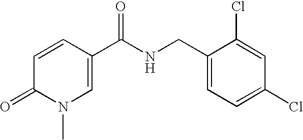

### 1-Methyl-6-oxo-1,6-dihydro-pyridine-3-carboxylic acid 2,4-dichloro-benzylamide

To a solution of N-(2,4-Dichloro-benzyl)-6-hydroxy-nicotinamide (0.29 g, 1.0 mmol) and potassium carbonate (0.15 g, 1.1 mmol) in 7.5 mL of acetonitrile is added methyl iodide (0.068 mL, 1.1 mmol) and the reaction is heated to 60 C. After 3 hours additional methyl iodide is added (0.068 mL, 1.1 mmol) and the reaction heated for 1 hour at 60 C. The reaction is brought to room temperature, concentrated in vacuo and taken up in ethyl acetate. The organic layer is washed with water, saturated sodium bicarbonate, brine, dried over magnesium sulfate, filtered and evaporated to dryness. The resulting solid is titurtated with ether/petrolium ether and purified on silica gel with dichlormethane/methanol as the eluent to give (0.113 g, 33%) of the title compound. LCMS: 312.98 (M+H⁺).

### EXAMPLE 18

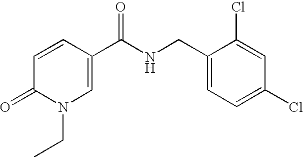

### 1-Ethyl-6-oxo-1,6-dihydro-pyridine-3-carboxylic acid 2,4-dichloro-benzylamide

The compound is prepared and purified using the procedure from example 17, starting from N-(2,4-Dichloro-benzyl)-6-hydroxy-nicotinamide (0.29 g, 1.0 mmol), ethyl iodide (0.16 mL, 2.0 mmol) and potassium carbonate (0.15 g, 1.1 mmol) in 7.5 mL of acetonitrile to provide the title compound (0.11 g, 32.6%). LCMS: 326.30 (M+H⁺).

### EXAMPLE 19

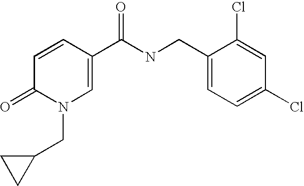

### 1-Cyclopropylmethyl-6-oxo-1,6-dihydro-pyridine-3-carboxylic acid 2,4-dichloro-benzylamide

The compound is prepared and purified using the procedure from example 17, starting from N-(2,4-Dichloro-benzyl)-6-hydroxy-nicotinamide (0.29 g, 1.0 mmol), cyclopropylmethylbromide (0.194 mL, 2.0 mmol) and potassium carbonate (0.15 g, 1.1 mmol) in 7.5 mL of acetonitrile to provide the title compound (0.248 g, 35.3%). LCMS: 352.30 (M+H⁺).

### EXAMPLE 20

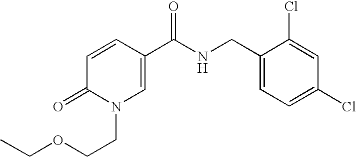

### 1-(2-Ethoxy-ethyl)-6-oxo-1,6-dihydro-pyridine-3-carboxylic acid 2,4-dichloro-benzylamide

The compound is prepared and purified using the procedure from example 17, starting from N-(2,4-Dichloro-benzyl)-6-hydroxy-nicotinamide (0.29 g, 1.0 mmol), bromoethoxyethane (0.194 mL, 2.0 mmol) and potassium carbonate (0.15 g, 1.1 mmol) in 7.5 mL of acetonitrile to provide the title compound (0.178 g, 24.1%). LCMS: 370.27 (M+H⁺).

### EXAMPLE 21

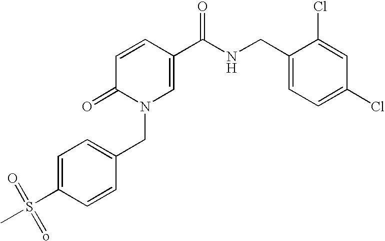

### 1-(4-Methanesulfonyl-benzyl)-6-oxo-1,6-dihydro-pyridine-3-carboxylic acid 2,4-dichloro-benzylamide

The compound is prepared and purified using the procedure from example 17, starting from N-(2,4-Dichloro-benzyl)-6-hydroxy-nicotinamide (0.29 g, 1.0 mmol), bromomethyl-4-methanesulfonylbenzene (0.498 g, 2.0 mmol) and potassium carbonate (0.15 g, 1.11 mmol) in 7.5 mL of acetonitrile to provide the title compound (0.068 g, 7.3%). LCMS: 466.17 (M+H⁺).

### EXAMPLE 22

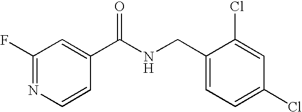

### N-(2,4-Dichloro-benzyl)-2-fluoro-isonicotinamide

The compound is prepared and purified using the procedure from example 1, starting from 2-fluoroisonicotininc acid (5.21 g, 37.5 mmol), 2,4 dichlorobenzylamine 5.0 mL, 37.5 mmol), 1-hydroxybenzotriazole (10.1 g, 75.0 mmol), 1-ethyl-3-(3-dimethylaminopropyl)-carbodiimide hydrochloride (14.3 g, 75.0 mmol) and diisopropylethylamine (20.1 mL, 112.0 mmol) N,N,dimethylformamide (190 mL) to provide the title compound (9.8 g, 87.4%). LCMS: 300.00 (M+H⁺).

### EXAMPLE 23

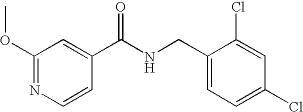

### N-(2,4-Dichloro-benzyl)-2-methoxy-isonicotinamide

The compound is prepared and purified using the procedure from example 3, starting from sodium hydride (60% in mineral oil, 70.0 mg 1.75 mmol), methanol (0.078 mL, 1.75 mmol), N-(2,4-Dichloro-benzyl)-2-fluoro-isonicotinamide (300 mg, 1.00 mmol), and N,N dimethylacetamide (3.0 mL) to provide the title compound (0.221 g, 70.8%). LCMS: 312.94 (M+H⁺).

### EXAMPLE 24

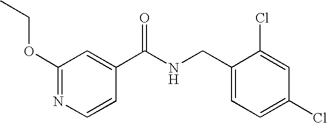

### N-(2,4-Dichloro-benzyl)-2-ethoxy-isonicotinamide

The compound is prepared and purified using the procedure from example 3, starting from sodium hydride (60% in mineral oil, 70.0 mg 1.75 mmol), ethanol (0.102 mL, 1.75 mmol), N-(2,4-Dichloro-benzyl)-2-fluoro-isonicotinamide (300 mg, 1.00 mmol), and N,N dimethylacetamide (3.0 mL) to provide the title compound (0.140 g, 42.9%). LCMS: 236.12 (M+H⁺).

### EXAMPLE 25

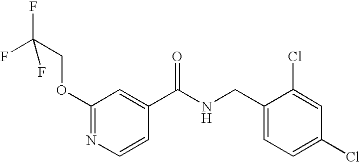

### N-(2,4-Dichloro-benzyl)-2-(2,2,2-trifluoro-ethoxy-isonicotinamide

The compound is prepared and purified using the procedure from example 3, starting from sodium hydride (60% in mineral oil, 50.0 mg 1.25 mmol), 2,2,2 trifluoroethanol (0.091 mL, 1.25 mmol), N-(2,4-Dichloro-benzyl)-2-fluoro-isonicotinamide (300 mg, 1.00 mmol), and N,N dimethylacetamide (3.0 mL) to provide the title compound (0.085 g, 22.4%). LCMS: 380.18 (M+H¹).

### EXAMPLE 26

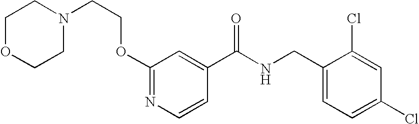

### N-(2,4-Dichloro-benzyl)-2-(2-morpholin-4-yl-ethoxy)-isonicotinamide

The compound is prepared and purified using the procedure from example 3, starting from sodium hydride (60% in mineral oil, 50.0 mg 1.25 mmol), 2-morpholin-4-yl ethanol (0.182 mL, 1.50 mmol), 1.25 mmol), N-(2,4-Dichloro-benzyl)-2-fluoro-isonicotinamide (300 mg, 1.00 mmol), and N,N dimethylacetamide (3.0 mL) to provide the title compound 0.212 g, 51.5%) LCMS: 411.31 (M+H⁺).

### EXAMPLE 27

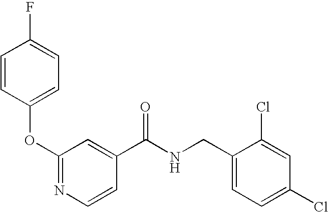

### N-(2,4-Dichloro-benzyl)-2-(4-fluoro-phenoxy)-isonicotinamide

The compound is prepared and purified using the procedure from example 3, starting from sodium hydride (60% in mineral oil, 50.0 mg 1.25 mmol), 4-fluorophenol (0.140 g, 1.25 mmol), N-(2,4-Dichloro-benzyl)-2-fluoro-isonicotinamide (300 mg, 1.00 mmol), and N,N dimethylacetamide (3.0 mL) to provide the title compound (0.17 g, 54.5%). LCMS: 392.13 (M+H⁺).

### EXAMPLE 28

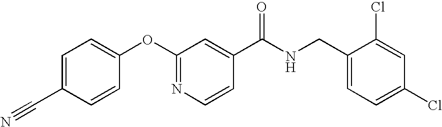

### 2-(4-Cyano-phenoxy)-N-(2,4-dichloro-benzyl)-isonicotinamide

The compound is prepared and purified using the procedure from example 3, starting from sodium hydride (60% in mineral oil, 50.0 mg 1.25 mmol), 4-cyanophenol (0.148 g, 1.25 mmol), N-(2,4-Dichloro-benzyl)-2-fluoro-isonicotinamide (300 mg, 1.00 mmol), and N,N dimethylacetamide (3.0 mL) to provide the title compound (0.034 g, 8.5%). LCMS: 399.19 (M+H⁺).

### EXAMPLE 29

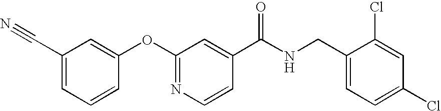

### 2-(3-Cyano-phenoxy)-N-(2,4-dichloro-benzyl)-isonicotinamide

The compound is prepared and purified using the procedure from example 3, starting from sodium hydride (60% in mineral oil, 50.0 mg 1.25 mmol), 3-cyanophenol (0.148 g, 1.25 mmol), N-(2,4-Dichloro-benzyl)-2-fluoro-isonicotinamide (300 mg, 1.00 mmol), and N,N dimethylacetamide (3.0 mL) to provide the title compound (0.100 g, 25.0%). LCMS: 399.19 (M+H⁺).

### EXAMPLE 30

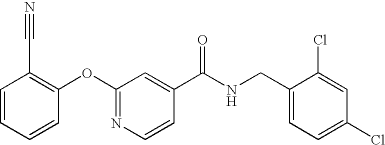

### 2-(2-Cyano-phenoxy)-N-(2,4-dichloro-benzyl)-isonicotinamide

The compound is prepared and purified using the procedure from example 3, starting from sodium hydride (60% in mineral oil, 50.0 mg 1.25 mmol), 2-cyanophenol (0.148 g, 1.25 mmol), N-(2,4-Dichloro-benzyl)-2-fluoro-isonicotinamide (300 mg, 1.00 mmol), and N,N dimethylacetamide (3.0 mL) to provide the title compound (0.091 g, 22.8%). LCMS: 399.19 (M+H⁺).

### EXAMPLE 31

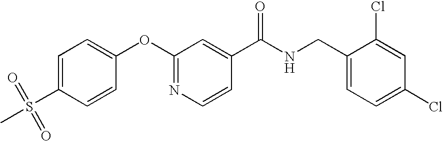

### N-(2,4-Dichloro-benzyl)-2-(4-methanesulfonyl-phenoxy)-isonicotinamide

The compound is prepared and purified using the procedure from example 3, starting from sodium hydride (60% in mineral oil 50.0 mg 1.25 mmol), 4-methylsulfonylphenol (0.215 g, 1.25 mmol), N-(2,4-Dichloro-benzyl)-2-fluoro-isonicotinamide (300 mg, 1.00 mmol), and N,N dimethylacetamide (3.0 mL) to provide the title compound (0.040 g, 8.8%). LCMS: 452.18 (M+H¹).

### EXAMPLE 32

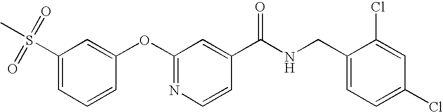

### N-(2,4-Dichloro-benzyl)-2-(3-methanesulfonyl-phenoxy-isonicotinamide

The compound is prepared and purified using the procedure from example 3, starting from sodium hydride (60% in mineral oil, 50.0 mg 1.25 mmol), 3-methylsulfonylphenol (0.215 g, 1.25 mmol, Bordwell, F. G.; et. al. J. Am. Chem. Soc. 1991, 113, 1736), N-(2,4-Dichloro-benzyl)-2-fluoro-isonicotinamide (300 mg, 1.00 mmol), and N,N dimethylacetamide (3.0 mL) to provide the title compound (0.133 g, 29.4%). LCMS: 452.18 (M+H⁺).

### EXAMPLE 33

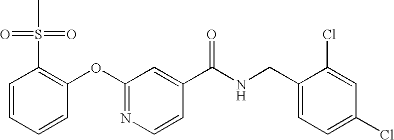

### N-(2,4-Dichloro-benzyl)-2-(2-methanesulfonyl-phenoxy)-isonicotinamide

The compound is prepared and purified using the procedure from example 3, starting from sodium hydride (60% in mineral oil, 50.0 mg 1.25 mmol), 2-methylsulfonylphenol (0.215 g, 1.25 mmol), N-(2,4-Dichloro-benzyl)-2-fluoro-isonicotinamide (300 mg, 1.00 mmol), and N,N dimethylacetamide (3.0 mL) to provide the title compound (0.085 g, 18.8%). LCMS: 452.18 (M+H⁺).

### EXAMPLE 34

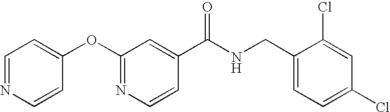

### N-(2,4-Dichloro-benzyl)-2-(pyridin-4-yloxy)-isonicotinamide

The compound is prepared and purified using the procedure from example 3, starting from sodium hydride (60% in mineral oil, 50.0 mg 1.25 mmol), 4-hydroxypyridine (0.120 g, 1.25 mmol), N-(2,4-Dichloro-benzyl)-2-fluoro-isonicotinamide (300 mg, 1.00 mmol), and N,N dimethylacetamide (3.0 mL) to provide the title compound (0.232 g, 61.8%). LCMS: 375.22 (M+H⁺).

### EXAMPLE 35

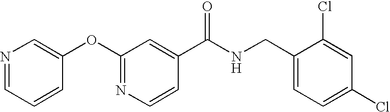

### N-(2,4-Dichloro-benzyl)-2-(pyridin-3-yloxy)-isonicotinamide

The compound is prepared and purified using the procedure from example 3, starting from sodium hydride (60% in mineral oil, 50.0 mg 1.25 mmol), 3-hydroxypyridine (0.120 g, 1.25 mmol), N-(2,4-Dichloro-benzyl)-2-fluoro-isonicotinamide (300 mg, 1.00 mmol), and N,N dimethylacetamide (3.0 mL) to provide the title compound (0.064 g, 17.1%). LCMS: 375.26 (M+H⁺).

### EXAMPLE 36

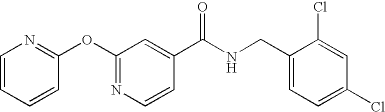

### N-(2,4-Dichloro-benzyl)-2-(pyridin-2-yloxy)-isonicotinamide

The compound is prepared and purified using the procedure from example 3, starting from sodium hydride (60% in mineral oil, 50.0 mg 1.25 mmol), 2-hydroxypyridine (0.120 g, 1.25 mmol), N-(2,4-Dichloro-benzyl)-2-fluoro-isonicotinamide (300 mg, 1.00 mmol), and N,N dimethylacetamide (3.0 mL) to provide the title compound (0.115 g, 30.6%). LCMS: 375.24 (M+H⁺).

### EXAMPLE 37

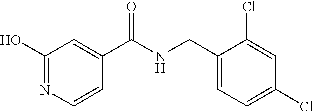

### N-(2,4-Dichloro-benzyl)-2-hydroxy-isonicotinamide

To 2-Benzyloxy-N-(2,4-dichloro-benzyl)-isonicotinamide (150 mg, 0.387 mmol) is added trifluororacetic acid (2.0 mL, 114 mmol), and the reaction stirred at room temperature for 16 hours. The excess trifluoroacetic acid is evaporated in vacuo and the resulting oil is treated with dietyl ether and a solid precipitated. The solid is collected washed with dietyl ether and dried to give the title compound (0.061 g, 38.3%) as the TFA salt. LCMS: 298.14 (M+H⁺).

### EXAMPLE 38

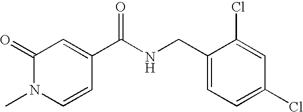

### 1-Methyl-2-oxo-1,2-dihydro-pyridine-4-carboxylic acid 2,4-dichloro-benzylamide

The compound is prepared and purified using the procedure from example 17, starting from N-(2,4-Dichloro-benzyl)-2-hydroxy-isonicotinamide (0.20 g, 0.67 mmol), methyliodide (0.063 mL, 1.0 mmol) and potassium carbonate (0.179 g, 1.38 mmol) in 6.3 mL of acetonitrile to provide the title compound (0.039 g, 12.5%). LCMS: 312.26 (M+H⁺).

### EXAMPLE 39

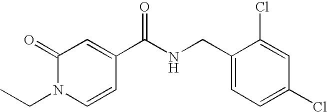

### 1-Ethyl-2-oxo-1,2-dihydro-pyridine-4-carboxylic acid 2,4-dichloro-benzylamide

The compound is prepared and purified using the procedure from example 17, starting from N-(2,4-Dichloro-benzyl)-2-hydroxy-isonicotinamide (0.20 g, 0.67 mmol), ethyliodide (0.079 mL, 1.0 mmol) and potassium carbonate (0.179 g, 1.38 mmol) in 6.3 mL of acetonitrile to provide the title compound (0.029 g, 8.9%). LCMS: 326.28 (M+H⁺).

### EXAMPLE 40

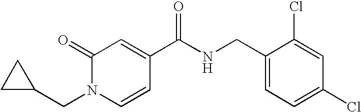

### 1-Cyclopropylmethyl-2-oxo-1,2-dihydro-pyridine-4-carboxylic acid 2,4-dichloro-benzylamide

The compound is prepared and purified using the procedure from example 17, starting from N-(2,4-Dichloro-benzyl)-2-hydroxy-isonicotinamide (0.20 g, 0.67 mmol), bromomethylcyclopropane (0.112 mL, 1.0 mmol) and potassium carbonate (0.179 g, 1.38 mmol) in 6.3 mL of acetonitrile to provide the title compound (0.0391 g, 8.4%). LCMS: 326.28 (M+H⁺).

### EXAMPLE 41

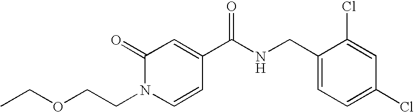

### 1-(2-Ethoxy-ethyl)-2-oxo-1,2-dihydro-pyridine-4-carboxylic acid 2,4-dichloro-benzylamide

The compound is prepared and purified using the procedure from example 17, starting from N-(2,4-Dichloro-benzyl)-2-hydroxy-isonicotinamide (0.20 g, 0.67 mmol), bromoethoxyethane (0.112 mL, 1.0 mmol) and potassium carbonate (0.179 g, 1.38 mmol) in 6.3 mL of acetonitrile to provide the title compound (0.031 g, 8.4%). LCMS: 370.28 (M+H⁺).

### EXAMPLE 42

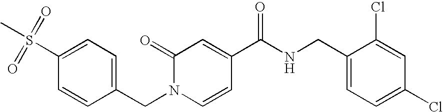

### 1-(4-Methanesulfonyl-benzyl)-2-oxo-1,2-dihydro-pyridine-4-carboxylic acid 2,4-dichloro-benzylamide

The compound is prepared and purified using the procedure from example 17, starting from N-(2,4-Dichloro-benzyl)-2-hydroxy-isonicotinamide (0.20 g, 0.67 mmol), bromomethyl-4-methanesulfonylbenzene (0.249 g, 1.0 mmol), and potassium carbonate (0.179 g, 1.38 mmol) in 6.3 mL of acetonitrile to provide the title compound (0.044 g, 9.5%). LCMS: 466.16 (M+H⁺).

### EXAMPLE 43

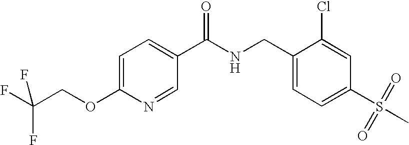

### N-(2-Chloro-4-methanesulfonyl-benzyl)-6-(2,2,2-trifluoro-ethoxy)-nicotinamide

### Step A: 2-Chloro-4-methanesulfonyl-benzamide

2-Chloro-4-methanesulfonyl-benzoic acid (21.0 g, 89.1 mmol) is suspended in acetonitrile (200 mL) and di-tert-butyl dicarbonate (27.0 g, 124 mmol) is added in one portion. The resulting mixture is stirred for 15 minutes and ammonium bicarbonate (79.10 mmol) followed by pyridine (11.2 mL 124 mmol) are added to the reaction. The reaction is stirred for 16 hours at room temperature and then the solvents evaporated in vacuo. The residue is triturated with 10% NaOH and water until the solutions are clear and then the solids washed with water and 5% ether in petrolium ether. The solid is collected and dried in vacuo to give the desired compound (17.6 g, 84%). LCMS: 234.03 (M+H⁺).

### Step B: 2-Chloro-4-methanesulfonyl-benzylamine hydrochloride

To a solution of borane in THF (1M, 120 mL, 120 mmol) is added the compound from Step A over 5 minutes. The resulting suspension is heated to reflux and reacted for 16 hours. The reaction is cooled with an ice bath and excess borane is quenched by the slow addition of 6N HCl. The addition of HCl is stopped after gas evolution ceases and the resulting white solid precipitate is collected by vacuum filtration. The white solid is washed with 6N HCl and tetrahydrofuran/diethylether (1:1) and dried in vacuo to yield the desired product (8.20 g, 88%) LCMS: 222.23 (M+H⁺).

### Step C: N-(2-Chloro-4-methanesulfonyl-benzyl)-6-(2,2,2-trifluoro-ethoxy)-nicotinamide

The compound is prepared and purified using the procedure from example 1, starting from 6-(2,2,2 trifluoroethoxy)nicotinic acid (0.175 g, 0.79 mmol), 2-Chloro-4-methanesulfonyl-benzylamine hydrochloride (0.173 g, 0.79 mmol), 1-hydroxybenzotriazole (0.220 g, 1.62 mmol), 1-ethyl-3-(3-dimethylaminopropyl)-carbodiimide hydrochloride (0.303 g, 1.62 mmol) and diisopropylethylamine (0.5 mL, 3.8 mmol) N,N,dimethylformamide (4.0 mL) to provide the title compound (0.260 g, 77.7%). LCMS: 343.23 (M+H⁺).

### EXAMPLE 44

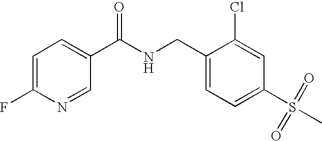

### N-(2-Chloro-4-methanesulfonyl-benzyl)-6-fluoro-nicotinamide

The compound is prepared and purified using the procedure from example 1, starting from 6-fluoronicotinic acid (1.1 g, 7.80 mmol), 2-Chloro-4-methanesulfonyl-benzylamine hydrochloride (2.0 g, 7.80 mmol), 1-hydroxybenzotriazole (2.11 g, 15.6 mmol), 1-ethyl-3-(3-dimethylaminopropyl)-carbodiimide hydrochloride (2.97 g, 15.6 mmol) and diisopropylethylamine (5.5 mL, 31.0 mmol) N,N,dimethylformamide (40 mL) to provide the title compound (2.32 g, 86.8%). LCMS: 343.23 (M+H⁺).

### EXAMPLE 45

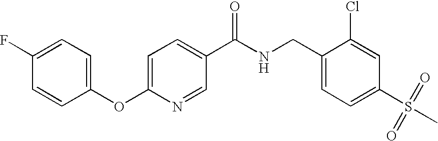

### N-(2-Chloro-4-methanesulfonyl-benzyl)-6-(4-fluoro-phenoxy)-nicotinamide

The compound is prepared and purified using the procedure from example 3, starting from sodium hydride (60% in mineral oil, 24.0 mg 0.600 mmol), 4-fluorophenol (0.067 g, 0.60 mmol), N-(2-Chloro-4-methanesulfonyl-benzyl)-6-fluoro-nicotinamide (0.171 g, 0.500 mmol), and N,N dimethylacetamide (3.0 mL) to provide the title compound (0.056 g, 25.8%). LCMS: 435.21 (M+H⁺).

### EXAMPLE 46

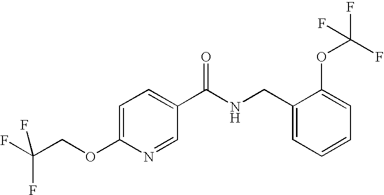

### 6-(2,2,2-Trifluoro-ethoxy)-N-(2-trifluoromethoxy-benzyl)-nicotinamide

The compound is prepared and purified using the procedure from example 1, starting from 6-(2,2,2 trifluoroethoxy)nicotinic acid (0.300 g, 1.35 mmol), 2-trifluoromethoxybenzylamine (0.267 g, 1.35 mmol), 1-hydroxybenzotriazole (0.378 g, 2.80 mmol), 1-ethyl-3-(3-dimethylaminopropyl)-carbodiimide hydrochloride (0.534 g, 2.80 mmol) and diisopropylethylamine (1.29 mL, 7.0 mmol). N,N,dimethylformamide (7.0 mL) to provide the title compound that is further purified on silica gel with dichloromethane/methanol as the eluent (0.350 g, 65.4%). LCMS: 395.23 (M+H⁺).

### EXAMPLE 47

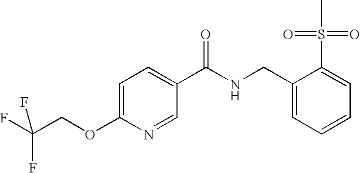

### N-(2-Methanesulfonyl-benzyl)-6-(2,2,2-trifluoro-ethoxy)-nicotinamide

The compound is prepared and purified using the procedure from example 1, starting from 6-(2,2,2 trifluoroethoxy)nicotinic acid (0.300 g, 1.35 mmol), 2-methylsulfonylbenzylamine hydrochloride (0.310 g, 1.40 mmol, WO2001038323), 1-hydroxybenzotriazole (0.378 g, 2.80 mmol), 1-ethyl-3-(3-dimethylaminopropyl)-carbodiimide hydrochloride (0.533 g, 2.80 mmol) and diisopropylethylamine (1.21 mL, 7.0 mmol) N,N,dimethylformamide (7.0 mL) to provide the title compound that is further purified on silica gel with dichloromethane/methanol as the eluent (0.326 g, 61.9%). LCMS: 389.24 (M+H⁺).

### EXAMPLE 48

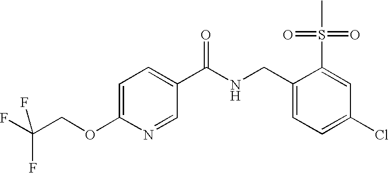

### N-(4-Chloro-2-methanesulfonyl-benzyl)-6-(2,2,2-trifluoro-ethoxy)-nicotinamide

### Step A: 4-Chloro-2-methylsulfanyl-benzamide

A solution of 2,4-dichlorobenzamide (5.00 g, 26.2 mmol) in dimethylformamide (131 mL) is treated with sodium thiomethoxide (3.20 g, 45.9 mmol) and heated at 60° C. After 2 h the reaction is cooled to room temperature and water is added. The solvent is removed in vacuo to give a white solid that is used without further purification.

### Step B: 4-Chloro-2-methylsulfanyl-benzylamine

Borane—THF (40 mL, 40 mmol) is added to the compound from Step A (2.66 g, 13.2 mmol) and allowed to stir for 16 h. The reaction is quenched by the slow addition of methanol. The solvents are removed from the reaction in vacuo, and resulting solid purified on silica gel using ethyl acetate/methanol as the eluent to give the desired product (1.20 g, 48%).

### Step C: 4-Chloro-2-methanesulfinyl-benzylamine para toluenesulfonic Acid Salt

A suspension of the compound from Step B (1.49 g, 7.94 mmol) in dichloromethane (80 mL) is treated with di-tert-butyl dicarbonate (1.73 g, 7.94 mmol) and triethylamine (1.10 mL, 7.94 mmol) and reacted until complete consumption as monitored by LC/MS. The solvents are removed in vacuo and the crude residue treated with dichloromethane (80 mL) and scuba (1.51 g, 8.73 mmol) and reacted until complete consumption as monitored by LC/MS. The solvents are removed in vacuo and the residue taken up in dichloromethane (5 mL) and trifluoroacetic acid (10 mL). After one hour para-toluene sulfonic acid is added (7.94 mmol) and the reaction is stirred for 30 minutes, The solvents are evaporated in vacuo to give a white solid (2.01 g, 64%), that was used without further purification.

### Step D: (4-Chloro-2-methanesulfonyl-benzyl)-carbamic acid tert-butyl ester

Aluminum oxide (9.68 g, 89.0 mmol) is added to water (2 mL) and stirred for 5 minutes. The compound from Step C (4.61 g, 11.7 mmol) is dissolved in chloroform (185 mL) and added to solution followed by oxone (19.3 g, 30.0 mmol). The reaction is heated at reflux for 16 hour cooled to room temperature, filtered and concentrated to give the desired product as colorless solid (2.01 g, 53.8%) that is in the next step without further purification.

### Step E: 4-Chloro-2-methanesulfonyl-benzylamine

The compound from Step D (2.01 g, 6.3 mmol) is dissolved in dichloromethane (10 mL) and trifluoroacetic acid (20 mL). The reaction is stirred for 1 hour and then the solvent removed in vacuo. Dichloromethane is added and evaporated three times and then the solid is dissolved in dichloromethane (10 mL) and para-toluene sulfonic acid (1.20 g 6.30 mmol) is added. The reaction is stirred for 1 hour then filtered and dried to give the desired product (2.00 g, 80.1%).

### Step F: N-(4-Chloro-2-methanesulfonyl-benzyl)-6-(2,2,2-trifluoro-ethoxy)-nicotinamide

The compound is prepared and purified using the procedure from example 1, starting from 6-(2,2,2 trifluoroethoxy)nicotinic acid (0.300 g, 1.35 mmol), 4-Chloro-2-methanesulfonyl-benzylamine (0.548.6 g, 1.40 mmol), 1-hydroxybenzotriazole (0.378 g, 2.80 mmol), 1-ethyl-3-(3-dimethylaminopropyl)-carbodiimide hydrochloride (0.533 g, 2.80 mmol) and diisopropylethylamine (1.21 mL, 7.0 mmol) N,N,dimethylformamide (7.0 mL) to provide the title compound that is further purified on silica gel with dichloromethane/methanol as the eluent (0.344 g, 60.0%). LCMS: 423.88 (M+H⁺).

### EXAMPLE 49

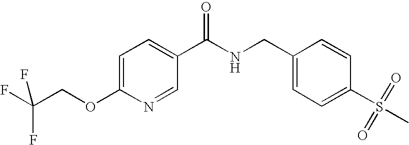

### N-(4-Methanesulfonyl-benzyl)-6-(2,2,2-trifluoro-ethoxy-nicotinamide

The compound is prepared and purified using the procedure from example 1, starting from 6-(2,2,2 trifluoroethoxy)nicotinic acid (0.300 g, 1.35 mmol), 4-methanesulfonyl-benzylamine (0.300 g, 1.35 mmol, Fuller, T.; et. al. J. Chem. Soc. 1945, 633.), 1-hydroxybenzotriazole (0.378 g, 2.80 mmol), 1-ethyl-3-(3-dimethylaminopropyl)-carbodiimide hydrochloride (0.533 g, 2.80 mmol) and diisopropylethylamine (1.21 mL, 7.0 mmol) N,N,dimethylformamide (7.0 mL) to provide the title compound that is further purified on silica gel with dichloromethane/methanol as the eluent (0.278 g, 52.8%). LCMS: 389.25 (M+H⁺).

### EXAMPLE 50

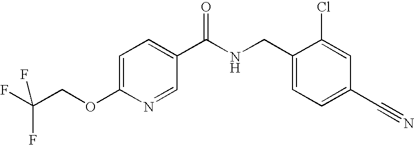

### N-(2-Chloro-4-cyano-benzyl)-6-(2,2,2-trifluoro-ethoxy)-nicotinamide

The compound is prepared and purified using the procedure from example 1, starting from 6-(2,2,2 trifluoroethoxy)nicotinic acid (0.148 g, 0.67 mmol), 4-aminomethyl-3-chlorobenzonitrile (0.112 g, 0.67 mmol, synthesized according to Gilbert, A. M.; et. al. J. Med Chem. 2000, 43, 1203-1214.), 1-hydroxybenzotriazole (0.182 g, 1.35 mmol), 1-ethyl-3-(3-dimethylaminopropyl)-carbodiimide hydrochloride (0.257 g, 1.35 mmol) and diisopropylethylamine (0.41 mL, 2.30 mmol) N,N,dimethylformamide (3.5 mL) to provide the title compound that is further purified on silica gel with dichloromethane/methanol as the eluent (0.220 g, 88.5%). LCMS: 370.03 (M+H⁺).

### EXAMPLE 51

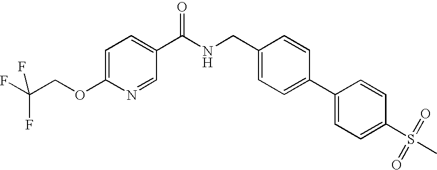

### N-(4′-Methanesulfonyl-biphenyl-4-ylmethyl)-6-(2,2,2-trifluoro-ethoxy)-nicotinamide

### Step A 4′-Methanesulfonyl-biphenyl-4-carbonitrile

4-methylsulfonylbornic acid (6.0 g, 30.0 mmol), 4bromobenzonitrile (4.91 g, 27.0 mmol), Pd₂(dba)₃ (0.180 g, 0.198 mmol) and [(t-bu)₃PH]BF₄ (0.120 g, 0.198 mmol) are weighed into a flask and the flask is sealed with a septum and purged with nitrogen for 5 minutes. Potassium flouride (5.22 g, 90.0 mmol) is added followed by THF (17 mL) and the reaction sealed, purged with nitrogen, and heated at 45° C. for 16 hours. The reaction is poured though a plug of silica gel and the silica gel washed with copius amound of THF. The solvents are evaporated in vacuo and the remaining solid is triturated with ethyl acetate/ether to provide the desired product (3.00 g, 38.9%). LCMS: 258.31 (M+H⁺).

### Step B (4′-Methanesulfonyl-biphenyl-4-ylmethyl)-carbamic Acid tert-butyl Ester

4′-Methanesulfonyl-biphenyl-4-carbonitrile (0.500 g, 1.94 mmol), di-t-butyl-dicarbamate (1.40 g, 6.41 mmol) and Pd/C (0.500 g, 10 mol % wet) are added to a parr apparatus and suspended in 25 mL of ethanol. The reaction vessel is purged thrice with hydrogen gas and then the pressure is increased to 60 psi and the solution shaken overnight. After 16 hours solution is filtered though a pad of celite and the celite washed with ethanol. The combined washings are evaporated in vacuo and the resulting solid purified on silica gel with hexanes/ethyl acetate as the eluent to give the desired product (0.390 g, 55.5%). LCMS: 362.45 (M+H⁺).

### Step C C-(4′-Methanesulfonyl-biphenyl-4-yl)methylamine

(4′-Methanesulfonyl-biphenyl-4-ylmethyl)-carbamic acid tert-butyl ester (0.390, 1.07 mmol) is dissolved in 5 mL of dioxane and HCl (4.0M in dioxane, 25 mL) is added to the reaction and the reaction stirred for 2 hours. The solid precipitate is collected and washed with ether to give the desired product (0.300 g, 93.4%). LCMS: 362.45 (M+H⁺).

### Step D

The compound is prepared and purified using the procedure from example 1, starting from 6-(2,2,2 trifluroethoxy)nicotinic acid (0.148 g, 0.67 mmol), C-(4′-Methanesulfonylbiphenyl-4-yl)methylamine hydrochloride (0.200 g, 0.67 mmol), 1-hydroxybenzotriazole (0.182 g, 1.35 mmol), 1-ethyl-3-(3-dimethylaminopropyl)-carbodiimide hydrochloride (0.257 g, 1.35 mmol) and diisopropylethylamine (0.41 mL, 2.30 mmol) N,N,dimethylformamide (3.5 mL) to provide the title compound that is further purified on silica gel with dichloromethane/methanol as the eluent (0.232 g, 74.5%). LCMS: 465.08 (M+H⁺).

### EXAMPLE 52

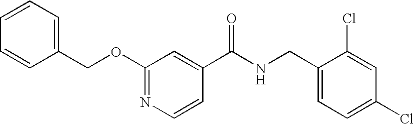

### 2-Benzyloxy-N-(2,4-dichloro-benzyl)-isonicotinamide

The compound is prepared and purified using the procedure from example 3, starting from sodium hydride (60% in mineral oil, 50.0 mg 1.25 mmol), benzylalcohol (0.108 mL, 1.05 mmol), N-(2,4-Dichloro-benzyl)-2-fluoro-isonicotinamide (300 mg, 1.00 mmol), and N,N dimethylacetamide (3.0 mL) to provide the title compound (0.150 g, 38.6%). LCMS: 388.92 (M+H⁺).

### EXAMPLE 53

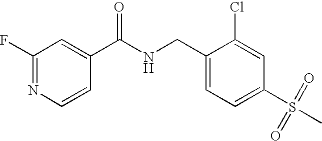

### N-(2-Chloro-4-methanesulfonyl-benzyl)-2-fluoro-isonicotinamide

The compound is prepared and purified using the procedure from example 1, starting from 2-fluoroisonicotinic acid (1.1 g, 7.80 mmol)₃ 2-Chloro-4-methanesulfonyl-benzylamine hydrochloride (2.0 g, 7.80 mmol), 1-hydroxybenzotriazole (2.11 g, 15.6 mmol), 1-ethyl-3-(3-dimethylaminopropyl)-carbodiimide hydrochloride (2.97 g, 15.6 mmol) and diisopropylethylamine (5.5 mL, 31.0 mmol) N,N,dimethylformamide (40 mL) to provide the title compound (2.00 g, 74.8%). LCMS: 343.23 (M+H⁺).

### EXAMPLE 54

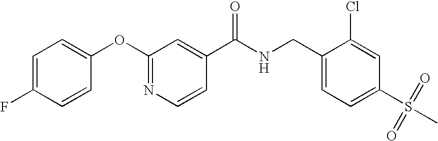

### N-(2-Chloro-4-methanesulfonyl-benzyl)-2-(4-fluoro-phenoxy)-isonicotinamide

The compound is prepared and purified using the procedure from example 3, starting from sodium hydride (60% in mineral oil, 50.0 mg 1.25 mmol), 4-fluorophenol (0.067 g, 0.112 mmol), N-(2-Chloro-4-methanesulfonyl-benzyl)-2-fluoro-isonicotinamide (0.171 g, 0.500 mmol), and N,N dimethylacetamide (3.0 mL) to provide the title compound (0.058 g, 26.7%). LCMS: 435.20 (M+H⁺).

### EXAMPLE 55

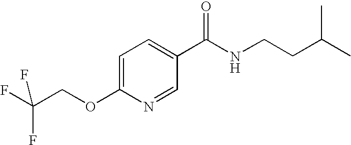

### N-(3-Methyl-butyl)-6-(2,2,2-trifluoro-ethoxy)-nicotinamide

The compound is prepared and purified using the procedure from example 1, starting from 2-fluoroisonicotinic acid (1.1 g, 7.80 mmol), 2-Chloro-4-methanesulfonyl-benzylamine hydrochloride (2.0 g, 7.80 mmol), 1-hydroxybenzotriazole (2.11 g, 15.6 mmol), 1-ethyl-3-(3-dimethylaminopropyl)-carbodiimide hydrochloride (2.97 g, 15.6 mmol) and diisopropylethylamine (5.5 mL, 31.0 mmol) N,N,dimethylformamide (40 mL) to provide the title compound (2.00 g, 74.8%). LCMS: 343.23 (M+H⁺).

### EXAMPLE 56

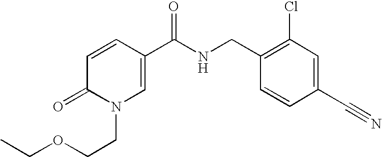

### 1-(2-Ethoxy-ethyl)-6-oxo-1,6-dihydro-pyridine-3-carboxylic acid 2-chloro-4-cyano-benzylamide

The compound is prepared and purified using the procedure from example 1, starting from 1-(2-Ethoxyethyl)-6-oxo-1,6-dihydropyridine-3-carboxylic acid (0.200 g, 0.947 mmol), C-(4′-4-aminomethyl-3-chlorobenzonitrile (0.157 g, 0.946 mmol, from example 50), 1-hydroxybenzotriazole (0.256 g, 1.89 mmol), 1-ethyl-3-(3-dimethylaminopropyl)-carbodiimide hydrochloride (0.361 g, 1.89 mmol) and diisopropylethylamine (0.595 mL, 3.31 mmol) N,N,dimethylformamide (4.2 mL) to provide the title compound that is further purified on silica gel with dichloromethane/methanol as the eluent (0.153 g, 44.9%). LCMS: 360.09 (M+H⁺).

### EXAMPLE 57

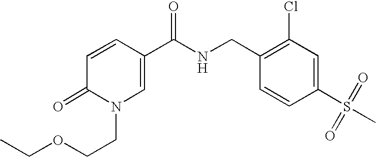

### 1-(2-Ethoxy-ethyl-6-oxo-1,6-dihydro-pyridine-3-carboxylic acid 2-chloro-4-methanesulfonyl-benzylamide

The compound is prepared and purified using the procedure from example 1, starting from 1-(2-Ethoxyethyl)-6-oxo-1,6-dihydropyridine-3-carboxylic acid (0.200 g, 0.947 mmol), C-(4′-4-aminomethyl-3-chlorobenzonitrile (0.252 g, 0.946 mmol), 1-hydroxybenzotriazole (0.256 g, 1.89 mmol), 1-ethyl-3-(3-dimethylaminopropyl)-carbodiimide hydrochloride (0.361 g, 1.89 mmol) and diisopropylethylamine (0.595 mL, 3.31 mmol) N,N,dimethylformamide (4.2 mL) to provide the title compound that is further purified on silica gel with dichloromethane/methanol as the eluent (0.143 g, 44.9%) LCMS: 413.05 (M+H⁺).

### EXAMPLE 58

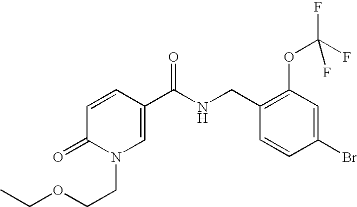

### 1-(2-Ethoxy-ethyl)-6-oxo-1,6-dihydro-pyridine-3-carboxylic acid 4-bromo-2-trifluoromethoxy-benzylamide

### Step A 4-Bromo-2-trifluoromethoxybenzaldehyde

4-Bromo-2 trifluoromethoxyiodobenzene (17.0 g, 46.0 mmol) is dissolved in 400 mL of THF and cooled to −78° C. Butyl lithium (1.5M in hexanes, 29.3 mL, 47.0 mmol) is added drop wise to the cooled solution over the course of 30 minutes. The reaction is stirred for an addition 30 minutes at −78° C. and then warmed to 0° C. and stirred for an additional 3 hours. The reaction is warmed to room temperature and stirred for 2 hours and is quenched by the slow addition of water. The solvents are removed in vacuo and the oil taken up in ethyl acetate and washed with 1N HCl, water, and brine. The ethyl acetate is dried (magnesium sulfate), filtered and evaporated to dryness to give a red solid that is used without further purification (9.80 g, 78.6%)

### Step B 4-Bromo-2-trifluoromethoxybenzylamine hydrochloride

4-Bromo-2-trifluoromethoxybenzaldehyde (8.20 g, 30.0 mmol) is dissolved in 150 mL of methanol (7N ammonia) and stirred overnight. After 16 hours the reaction is treated with sodium borahydride (2.48 g, 65.0 mmol) and stirred at room temperature for 3 hours. The solvents are then evaporated and the residue taken up slowly in 5% HCl and washed with diethyl ether. The aqueous layer is basified with sodium hydroxide and the product extracted with dichloromethane. The dichloromethane is dried with magnesium sulfate, filtered and evaporated to dryness to give an oil that is taken up in diethyl ether and treated with HCl (4N in ether) at which time a solid precipitates. The solid is collected was washed with ether and hexanes to give the desired product (7.20 g, 77.1%). LCMS: 271.19 (M+H⁺).

### Step C

The compound is prepared and purified using the procedure from example 1, starting from 1-(2-Ethoxyethyl)-6-oxo-1,6-dihydropyridine-3-carboxylic acid (0.689 g, 3.26 mmol), 4-Bromo-2-trifluoromethoxybenzylamine hydrochloride (1.00 g, 3.26 mmol), 1-hydroxybenzotriazole (0.882 g, 6.52 mmol), 1-ethyl-3-(3-dimethylaminopropyl)-carbodiimide hydrochloride (1.24 g, 6.52 mmol) and diisopropylethylamine (2.05 mL, 11.0 mmol) N,N,dimethylformamide (10.0 mL) to provide the title compound that is further purified on silica gel with dichloromethane/methanol as the eluent (1.20 g, 79.4%). LCMS: 464.13 (M+H⁺).

### EXAMPLE 59

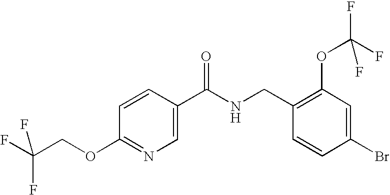

### N-(4-Bromo-2-trifluoromethoxy-benzyl)-6-(2,2,2-trifluoro-ethoxy)-nicotinamide

The compound is prepared and purified using the procedure from example 1, starting from 1-(2-6-(2,2,2-trifluoro-ethoxy)-nicotinic acid (0.360 g, 1.63 mmol), 4-Bromo-2-trifluoromethoxybenzylamine hydrochloride (0.500 g, 1.63 mmol, example 59), 1-hydroxybenzotriazole (0.441 g, 3.26 mmol), 1-ethyl-3-(3-dimethylaminopropyl)-carbodiimide hydrochloride (0.622 g, 3.26 mmol) and diisopropylethylamine (1.02 mL, 5.70 mmol) N,N,dimethylformamide (10.0 mL) to provide the title compound that is further purified on silica gel with dichloromethane/methanol as the eluent (0.655 g, 84.9%). LCMS: 474.06 (M+H⁺).

High Throughput Synthesis of benzamide sEH inhibitors:

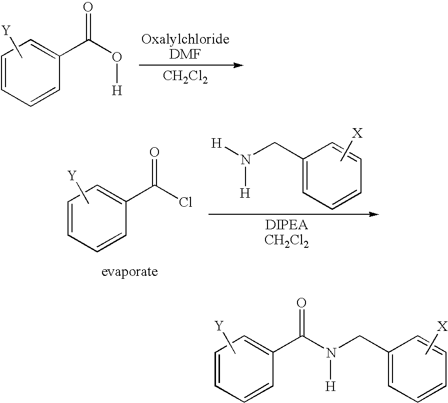

The acids (0.125 mmol) are weighed into individual reaction tubes and dissolved or suspended in 500 μL of CH₂Cl₂. Oxalyl chloride (0.163 mmol, 82 μL of a 2M solution in CH₂Cl₂, 1.3 eq.) is then added followed by 10 μL of DMF. The reactions are shaken at room temp for 1 hour, then the solvent is removed in vacuo. To each of the residues is added fresh CH₂Cl₂ (500 μL) followed by a solution of benzylamine (0.125 mmol) and diisopropylethylamine (0.188 mmol, 24 mg, 33 μL, 1.5 eq) in 500 μL CH₂Cl₂. For one benzylamine (2-Chloro-4-methanesulfonyl-benzylamine), the starting amine is supplied as the hydrochloride salt, so an additional 0.188 mmol diisopropylethylamine is used. The resultant reactions are shaken overnight at room temp.

After shaking for ˜15 hours, the final compounds are purified by passing the reaction mixtures through SPE cartridges containing 200 mg BondElut NH₂ and 200 mg BondElut CBA (Varian, Part #7553502C). The compounds are eluted with CH₂Cl₂ (5×500 μL) into pretared vials. The solvent is removed in vacuo and compounds that are found to be >80% product by LCMS (ELSD and UV) and ¹H NMR are submitted for biological testing.

Some samples are found to contain diisopropylethylamine hydrochloride by ¹H NMR. To these samples is added 500 μL CH₂Cl₂ and the material is refiltered through fresh SPE cartridges containing 200 mg BondElut NH₂ and 200 mg BondElut CBA (Varian, Part #7553502C) and eluted as above. The solvent is removed in vacuo and compounds that are found to be >80% product by LCMS (ELSD and UV) and ¹H NMR are submitted for biological testing.

### LC/MS for First Generation Library Compounds:

| Structure | Mw | MW from LCMS |
|---|---|---|
| 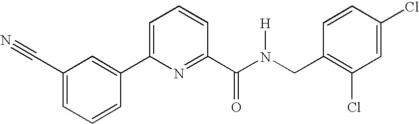 | 382.2 | 383 |
| 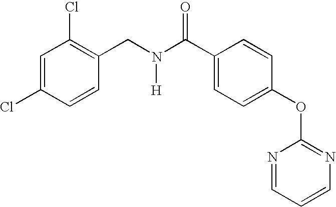 | 374.2 | 375 |
| 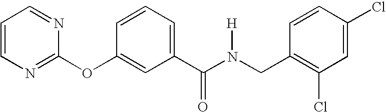 | 374.2 | 375 |
| 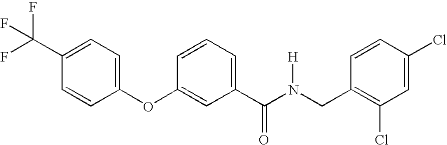 | 440.2 | 441 |
| 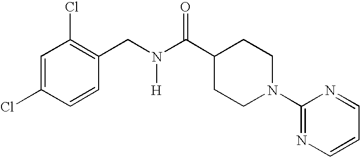 | 365.3 | 366 |
| 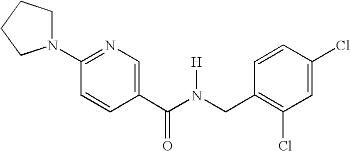 | 350.2 | 351 |
| 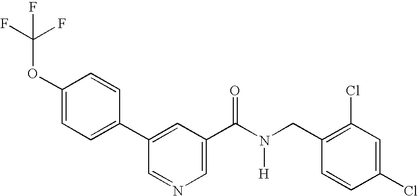 | 441.2 | 442 |
| 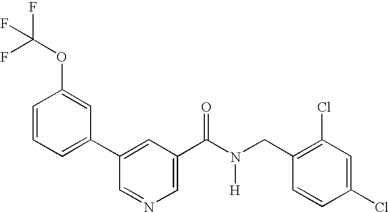 | 441.2 | 442 |
| 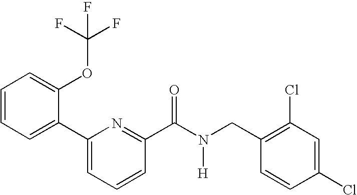 | 441.2 | 442 |
| 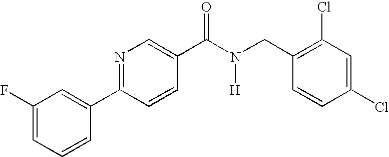 | 375.2 | 376 |
| 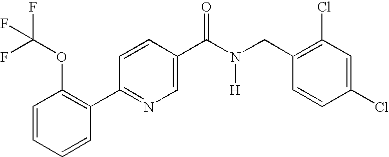 | 441.2 | 442 |
| 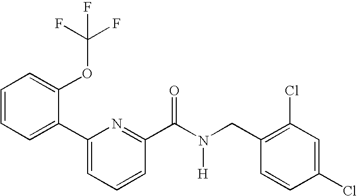 | 441.2 | 442 |
| 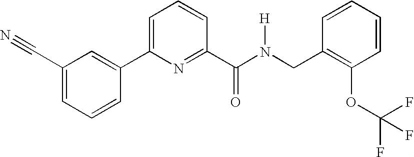 | 397.4 | 398 |
| 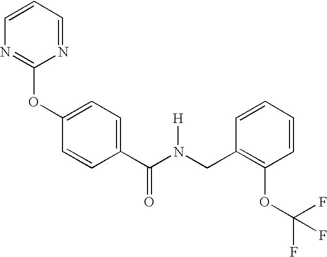 | 389.3 | 390 |
| 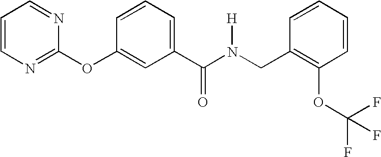 | 389.3 | 390 |
| 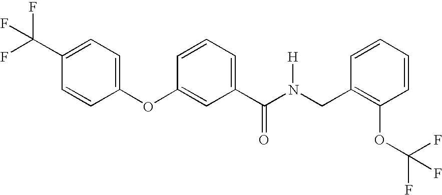 | 455.4 | 456 |
| 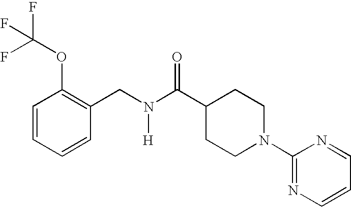 | 380.4 | 381 |
| 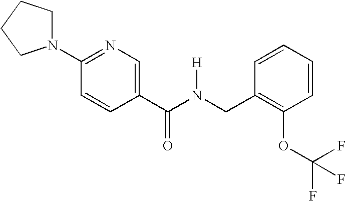 | 365.4 | 366 |
| 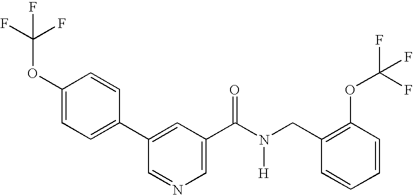 | 456.3 | 457 |
| 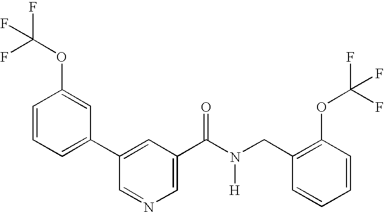 | 456.3 | 457 |
| 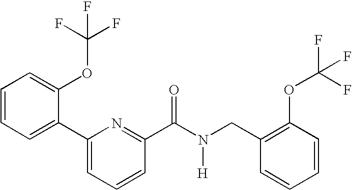 | 456.3 | 457 |
| 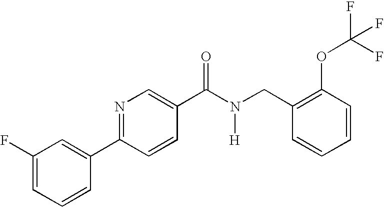 | 390.3 | 391 |
| 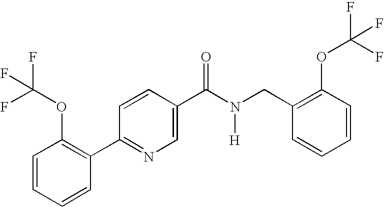 | 456.3 | 457 |
| 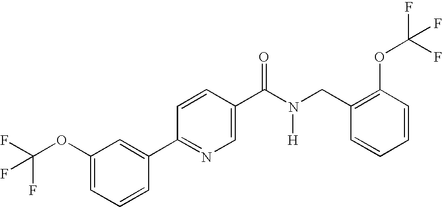 | 456.3 | 457 |
| 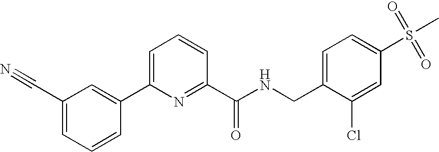 | 425.9 | 426 |
| 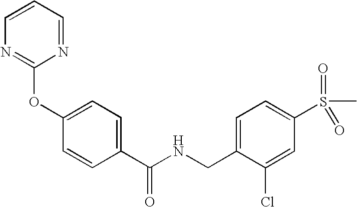 | 417.9 | 418 |
| 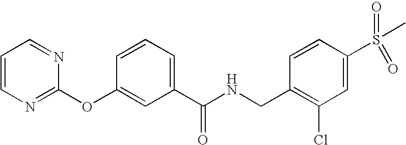 | 417.9 | 418 |
| 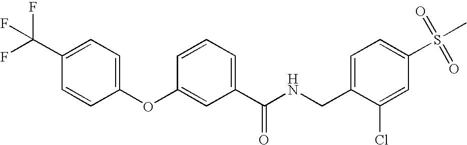 | 483.9 | 484 |
| 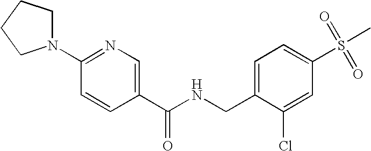 | 393.9 | 394 |
| 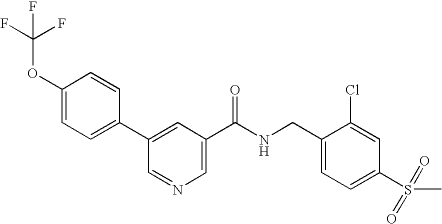 | 484.9 | 485 |
| 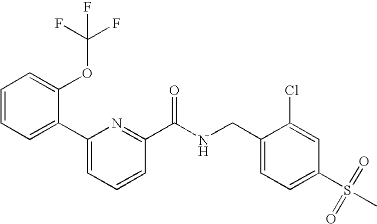 | 484.9 | 485 |
| 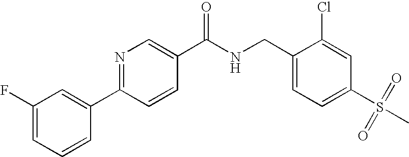 | 418.9 | 419 |
| 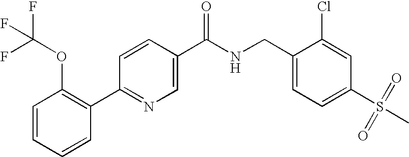 | 484.9 | 485 |
| 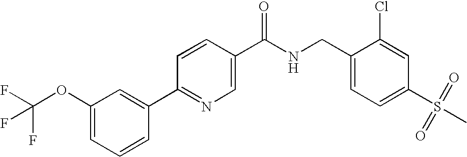 | 484.9 | 485 |
| 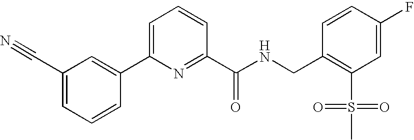 | 409.4 | 410 |
| 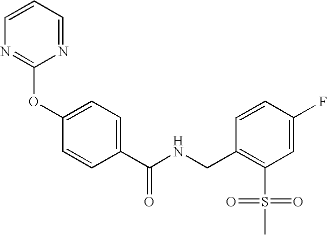 | 401.4 | 402 |
| 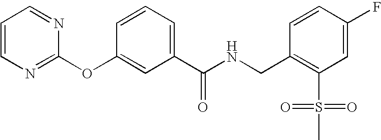 | 401.4 | 402 |
| 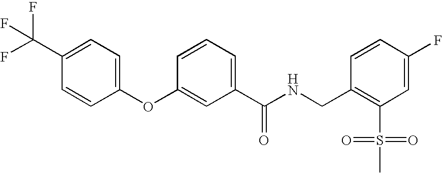 | 467.4 | 468 |
| 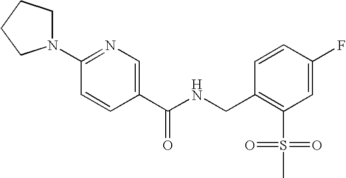 | 377.4 | 378 |
| 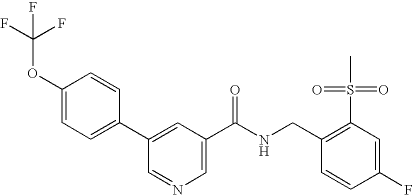 | 468.4 | 469 |
| 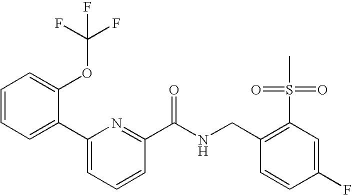 | 468.4 | 469 |
| 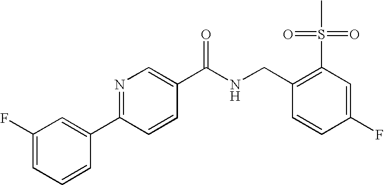 | 402.4 | 403 |
| 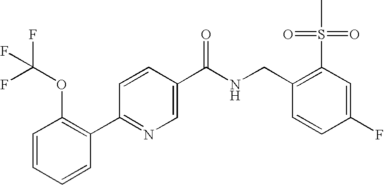 | 468.4 | 469 |
| 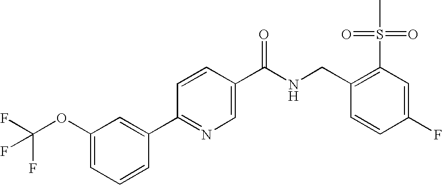 | 468.4 | 469 |
| 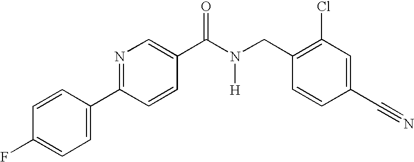 | 365.8 | 366 |
| 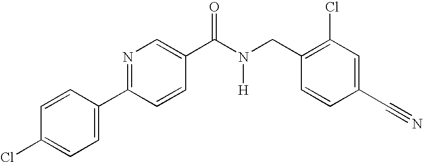 | 382.2 | 383 |
| 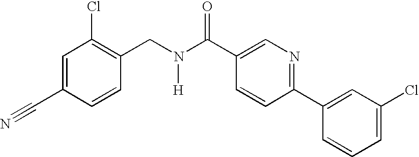 | 382.2 | 383 |
| 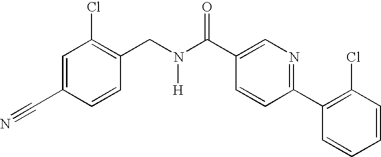 | 382.2 | 383 |
| 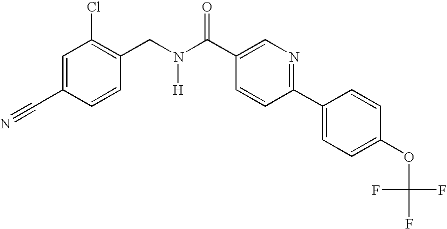 | 431.8 | 432 |
| 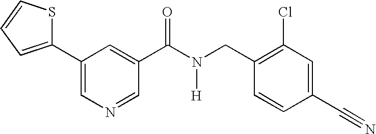 | 353.8 | 354 |
| 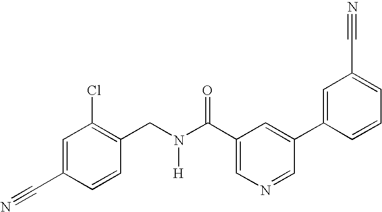 | 372.8 | 373 |
| 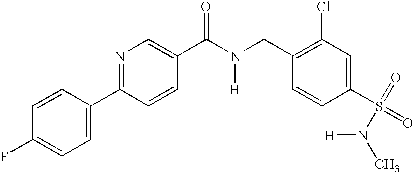 | 433.9 | 434 |
| 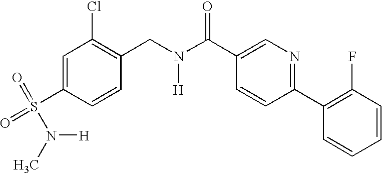 | 433.9 | 434 |
| 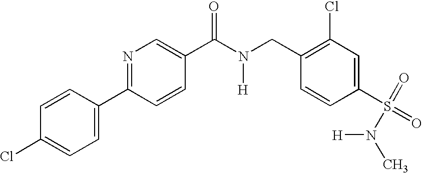 | 450.3 | 451 |
| 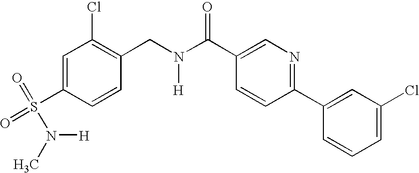 | 450.3 | 451 |
| 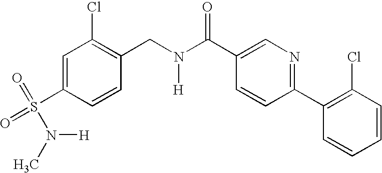 | 450.3 | 451 |
| 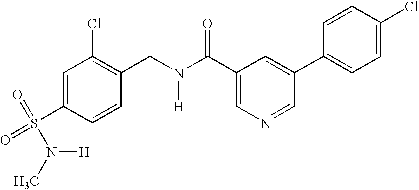 | 450.3 | 451 |
| 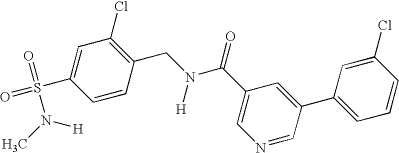 | 450.3 | 451 |
| 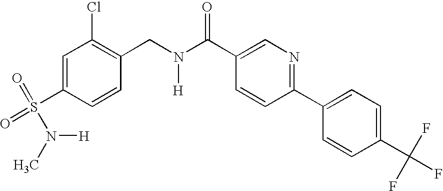 | 483.9 | 484 |
| 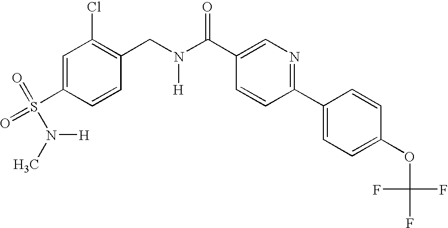 | 499.9 | 500 |
| 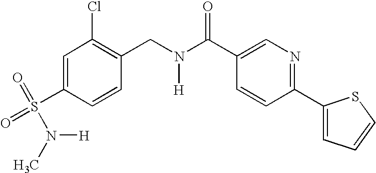 | 421.9 | 423 |
| 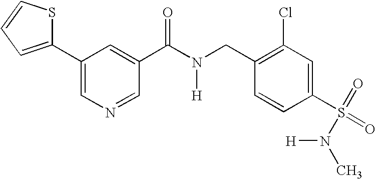 | 421.9 | 423 |
| 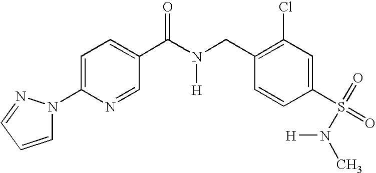 | 405.9 | 406 |
| 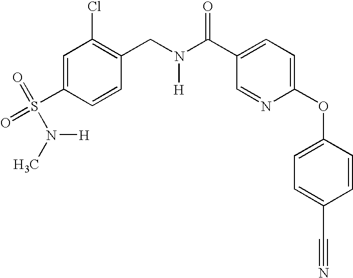 | 456.9 | 458 |
| 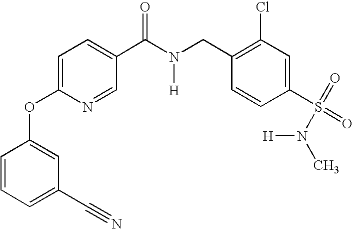 | 456.9 | 458 |
| 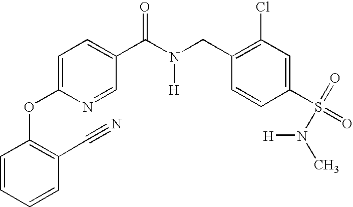 | 456.9 | 458 |
| 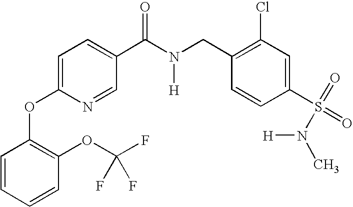 | 515.9 | 516 |
| 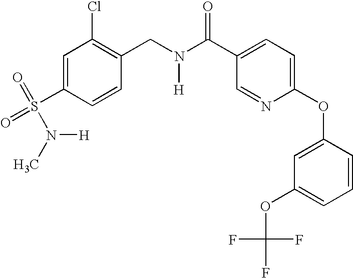 | 515.9 | 516 |
| 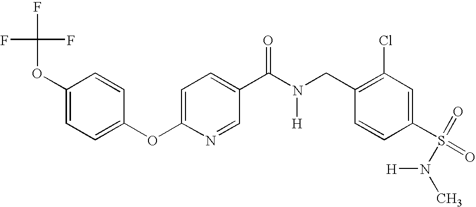 | 515.9 | 516 |
| 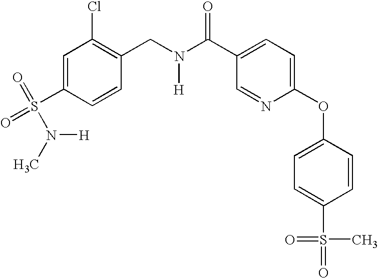 | 510.0 | 511 |
| 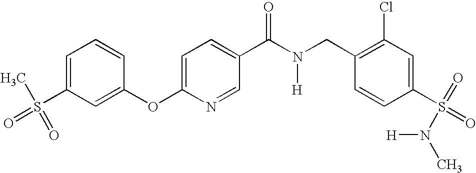 | 510.0 | 511 |
| 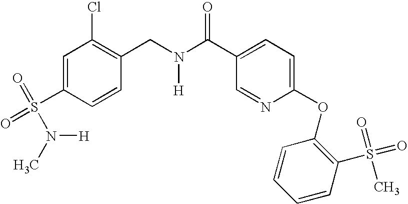 | 510.0 | 511 |
| 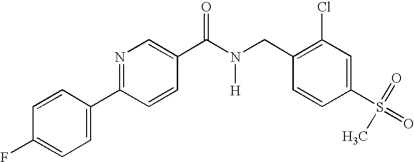 | 418.9 | 419 |
| 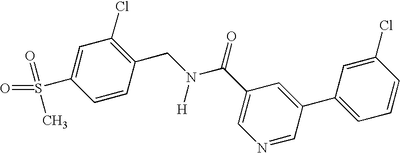 | 435.3 | 436 |
| 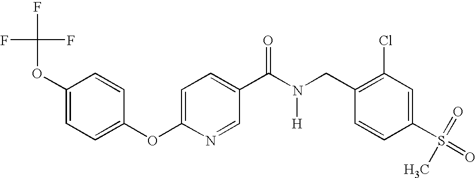 | 500.9 | 501 |
| 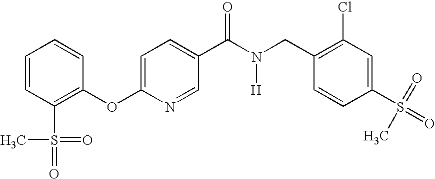 | 495.0 | 496 |
| 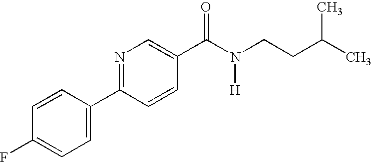 | 286.3 | 287 |
| 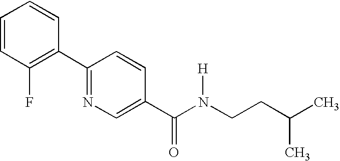 | 286.3 | 287 |
| 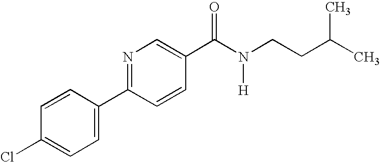 | 302.8 | 303 |
| 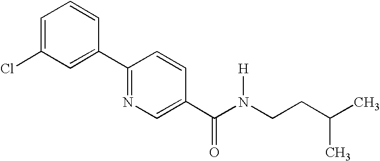 | 302.8 | 303 |
| 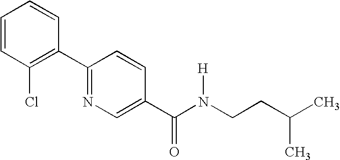 | 302.8 | 303 |
| 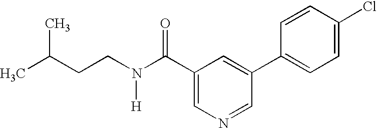 | 302.8 | 303 |
| 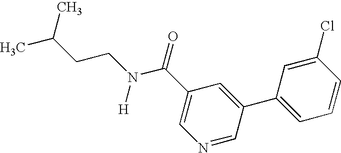 | 302.8 | 303 |
| 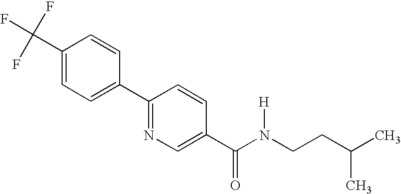 | 336.4 | 337 |
| 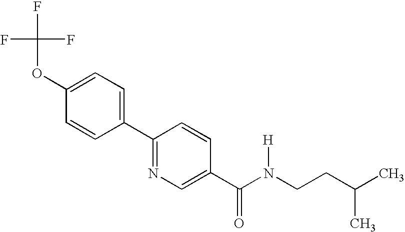 | 352.4 | 353 |
| 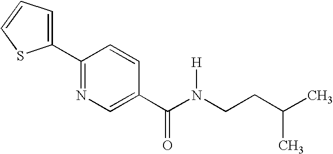 | 274.4 | 275 |
| 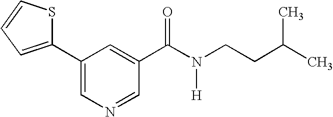 | 274.4 | 275 |
| 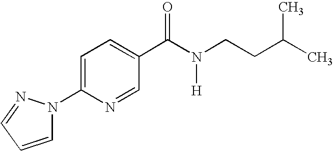 | 258.3 | 259 |
| 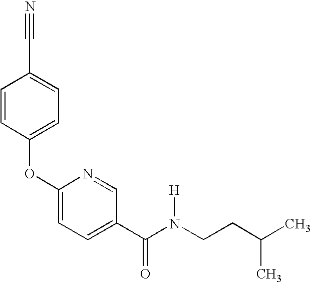 | 309.4 | 310 |
| 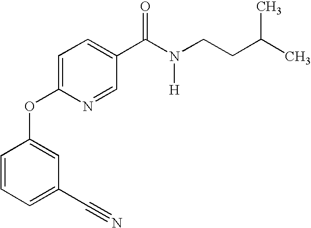 | 309.4 | 310 |
| 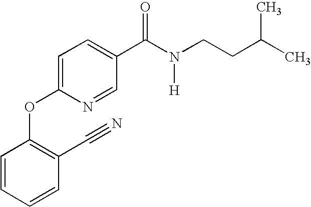 | 309.4 | 310 |
| 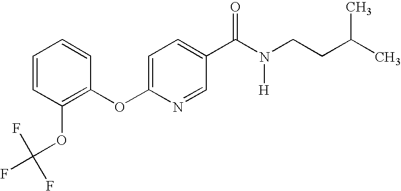 | 368.4 | 369 |
| 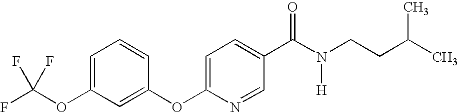 | 368.4 | 369 |
| 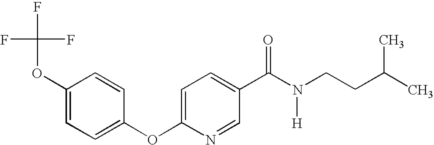 | 368.4 | 369 |
| 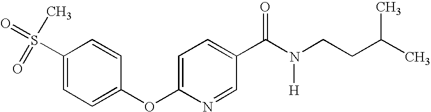 | 362.4 | 363 |
| 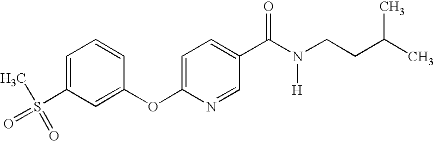 | 362.4 | 363 |
| 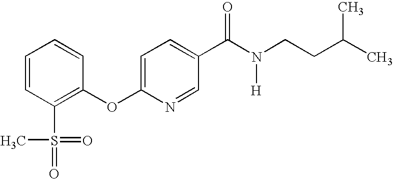 | 362.4 | 363 |
| 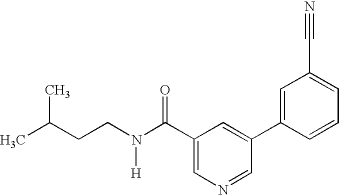 | 293.4 | 294 |
| 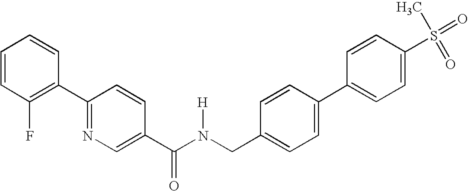 | 460.5 | 461 |
| 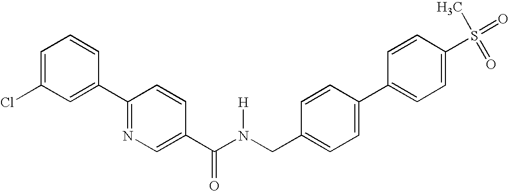 | 477.0 | 478 |
| 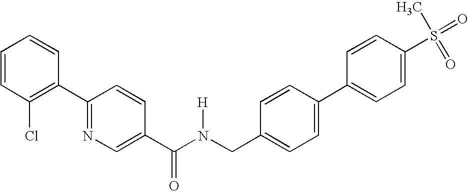 | 477.0 | 478 |
| 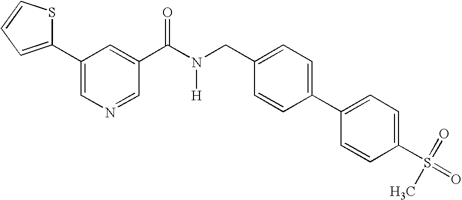 | 448.6 | 449 |
| 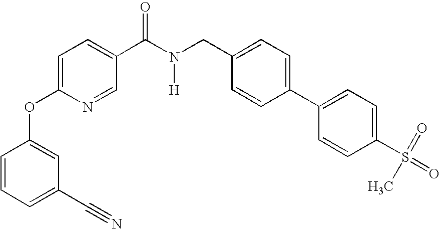 | 483.5 | 484 |
| 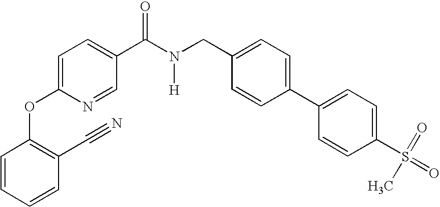 | 483.5 | 484 |
| 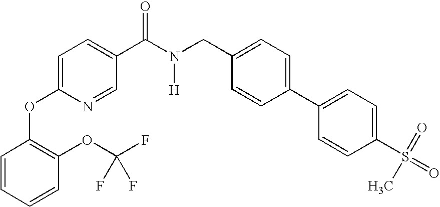 | 542.5 | 543 |
| 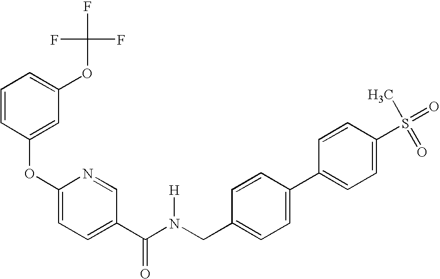 | 542.5 | 543 |
| 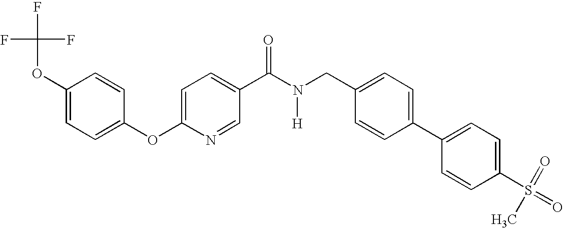 | 542.5 | 543 |
| 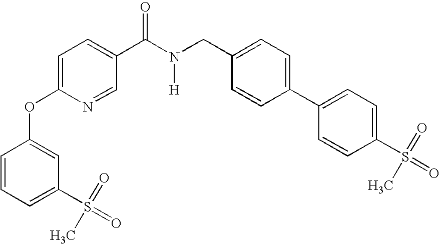 | 536.6 | 537 |
| 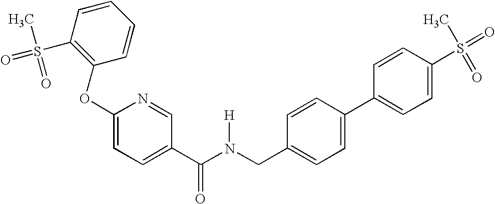 | 536.6 | 537 |
| 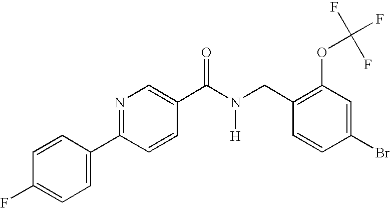 | 469.2 | 470 |
| 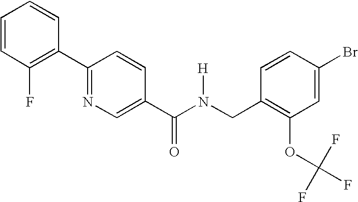 | 469.2 | 470 |
| 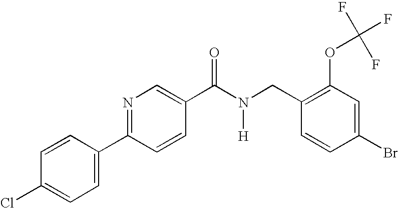 | 485.7 | 486 |
| 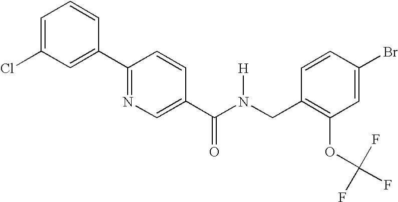 | 485.7 | 486 |
| 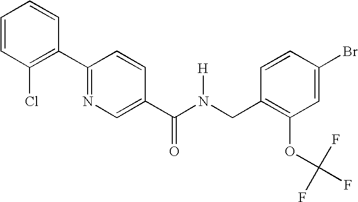 | 485.7 | 486 |
| 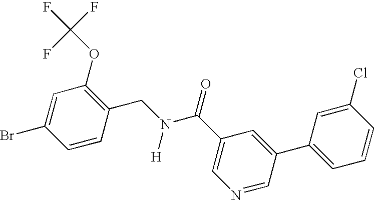 | 485.7 | 486 |
| 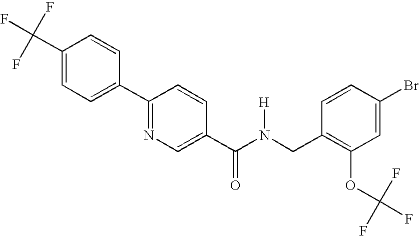 | 519.2 | 520 |
| 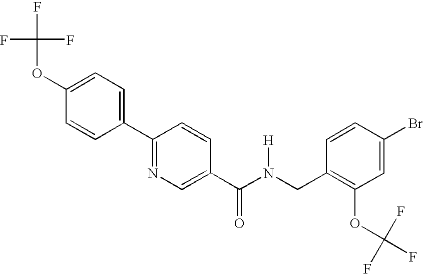 | 535.2 | 536 |
| 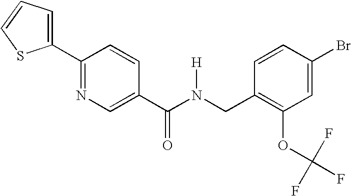 | 457.3 | 458 |
| 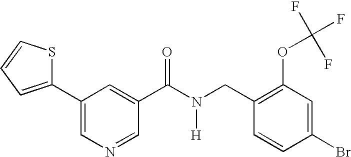 | 457.3 | 458 |
| 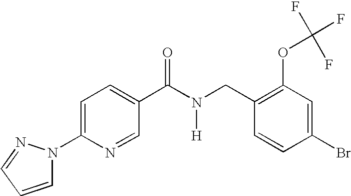 | 441.2 | 442 |
| 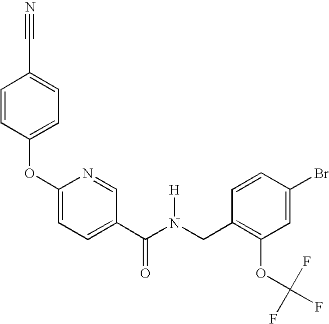 | 492.3 | 493 |
| 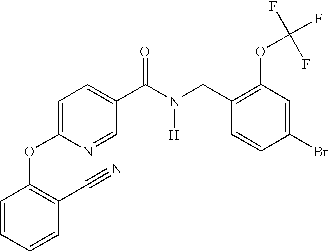 | 492.3 | 493 |
| 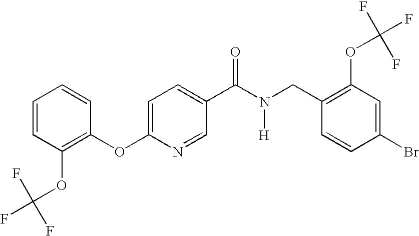 | 551.2 | 552 |
| 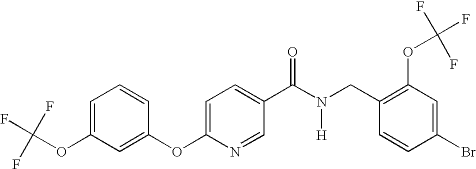 | 551.2 | 552 |
| 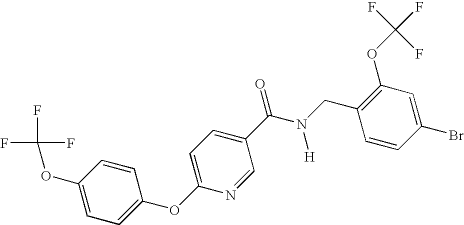 | 551.2 | 552 |
| 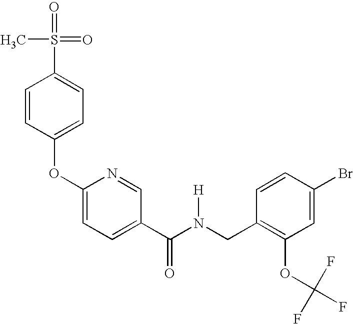 | 545.3 | 546 |
| 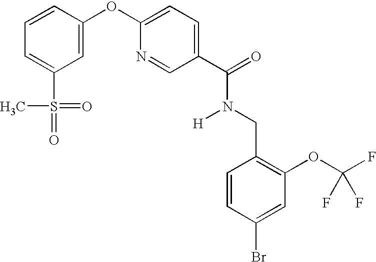 | 545.3 | 546 |
| 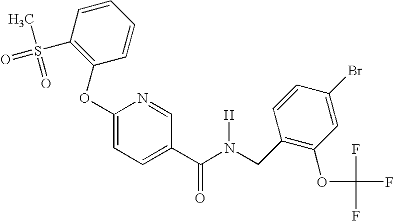 | 545.3 | 546 |
| 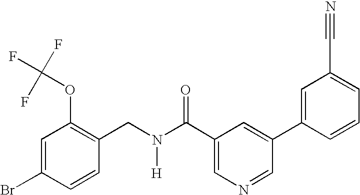 | 476.3 | 477 |
| 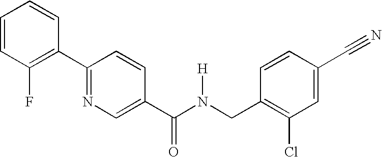 | 365.8 | 366 |
| 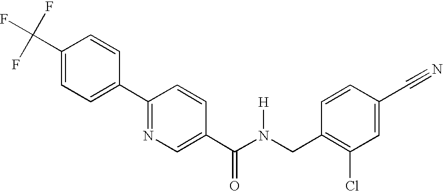 | 415.8 | 416 |
| 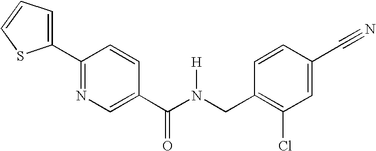 | 353.8 | 354 |
| 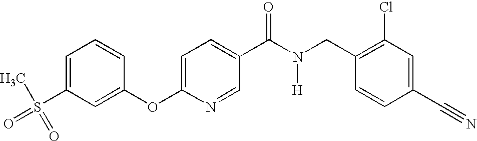 | 441.9 | 442 |
| 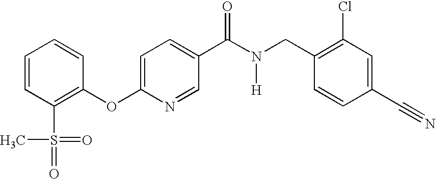 | 441.9 | 442 |
| 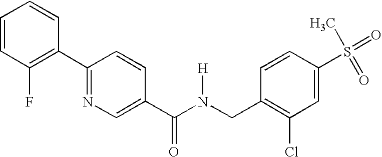 | 418.9 | 419 |
| 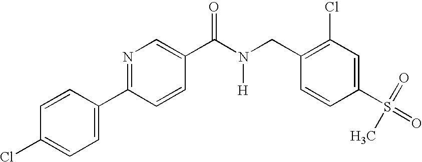 | 435.3 | 436 |
| 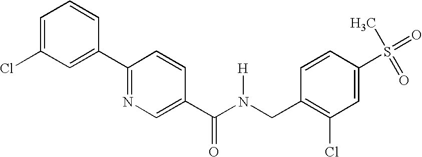 | 435.3 | 436 |
| 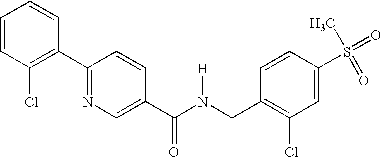 | 435.3 | 436 |
| 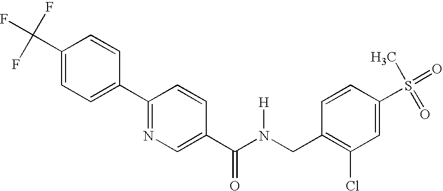 | 468.9 | 469 |
| 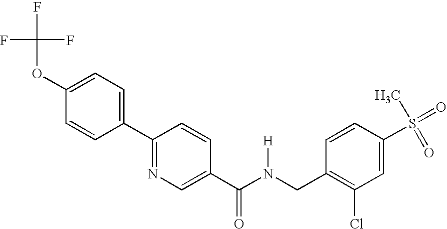 | 484.9 | 485 |
| 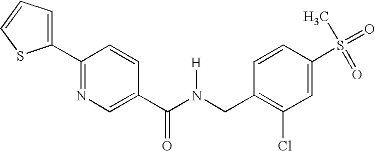 | 406.9 | 408 |
| 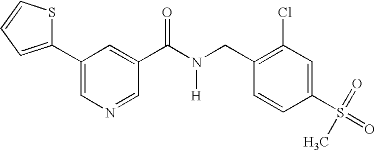 | 406.9 | 408 |
| 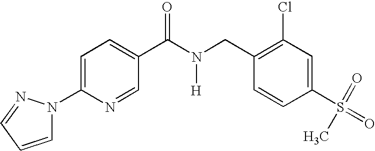 | 390.8 | 391 |
| 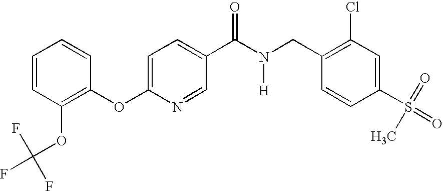 | 500.9 | 501 |
| 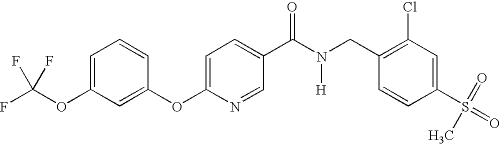 | 500.9 | 501 |
| 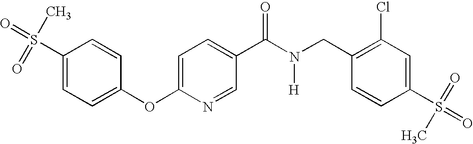 | 495.0 | 496 |
| 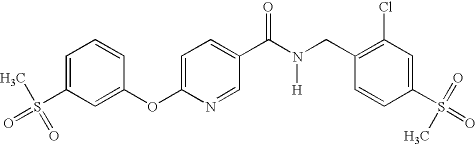 | 495.0 | 496 |
| 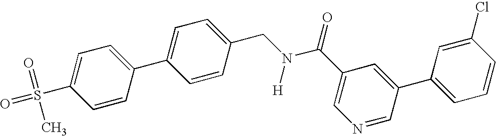 | 477.0 | 478 |
| 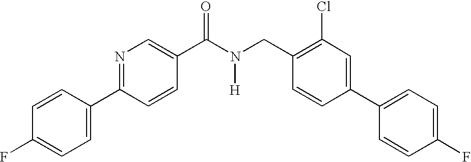 | 434.9 | 435 |
| 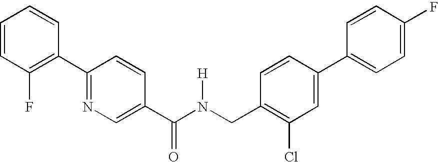 | 434.9 | 435 |
| 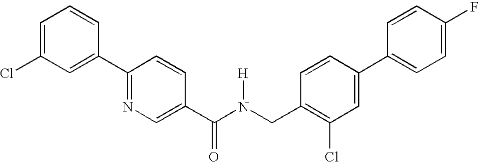 | 451.3 | 452 |
| 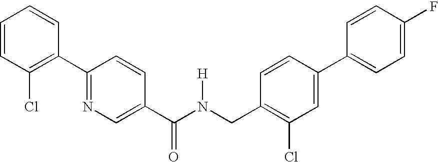 | 451.3 | 452 |
| 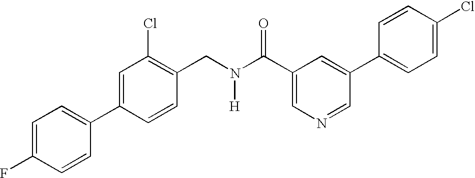 | 451.3 | 452 |
| 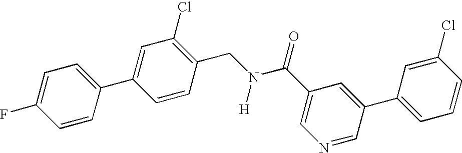 | 451.3 | 452 |
| 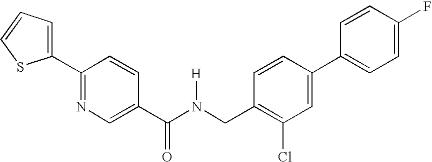 | 422.9 | 424 |
| 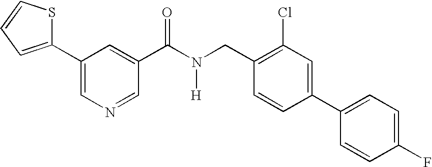 | 422.9 | 424 |
| 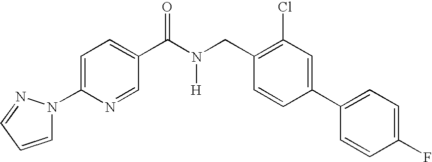 | 406.8 | 407 |
| 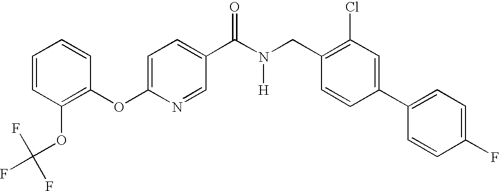 | 516.9 | 517 |
| 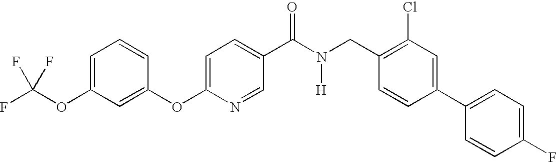 | 516.9 | 517 |
| 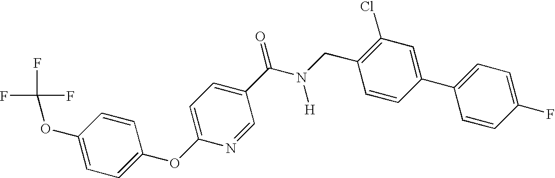 | 516.9 | 517 |
| 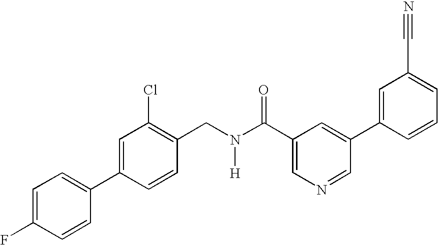 | 441.9 | 442 |
| 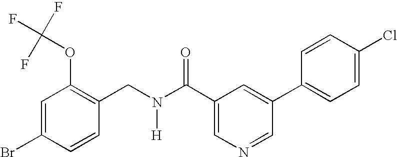 | 485.7 | 486 |
| 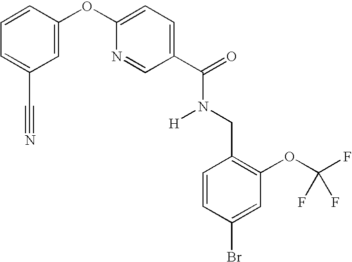 | 492.3 | 493 |
| 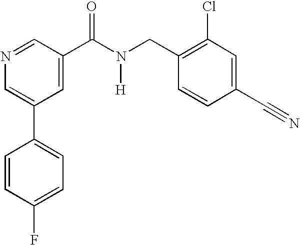 | 365.8 | 366 |
| 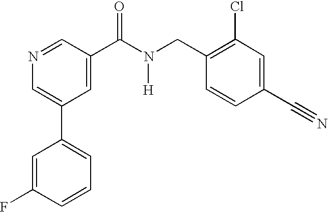 | 365.8 | 366 |
| 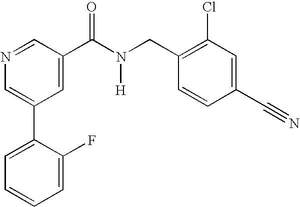 | 365.8 | 366 |
| 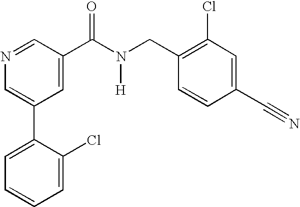 | 382.2 | 383 |
| 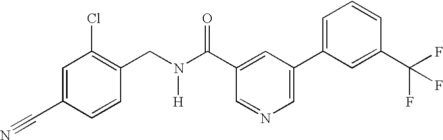 | 415.8 | 416 |
| 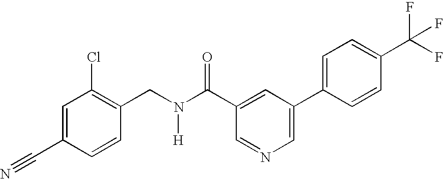 | 415.8 | 416 |
| 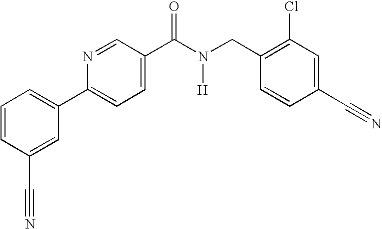 | 372.8 | 373 |
| 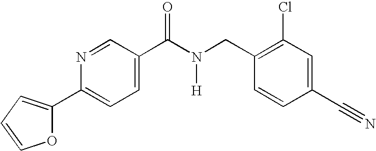 | 337.8 | 338 |
| 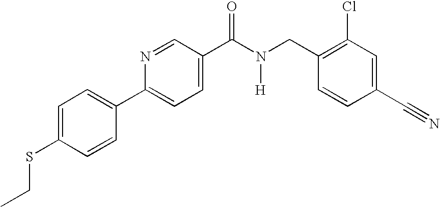 | 407.9 | 408 |
| 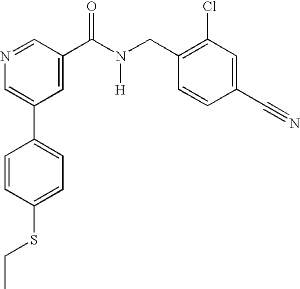 | 407.9 | 408 |
| 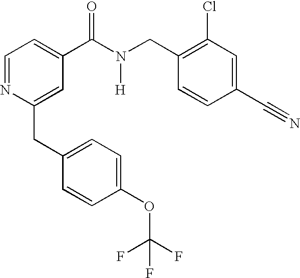 | 447.8 | 448 |
| 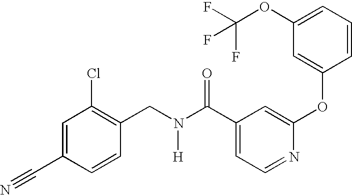 | 447.8 | 448 |
| 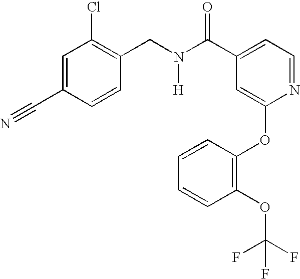 | 447.8 | 448 |
| 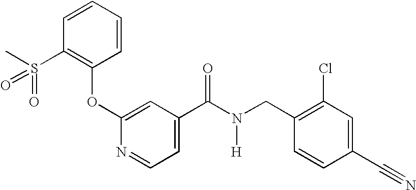 | 441.9 | 442 |
| 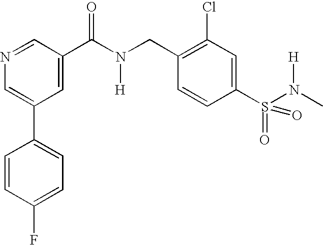 | 433.9 | 434 |
| 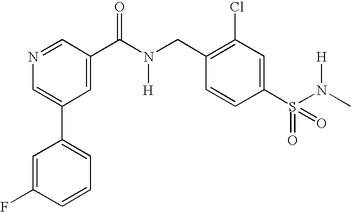 | 433.9 | 434 |
| 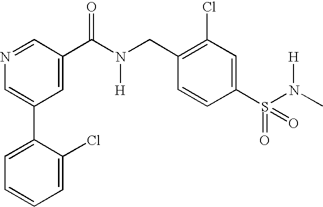 | 450.3 | 451 |
| 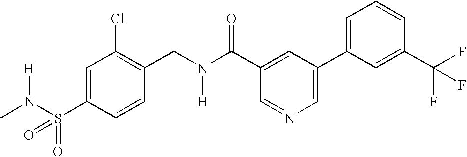 | 483.9 | 484 |
| 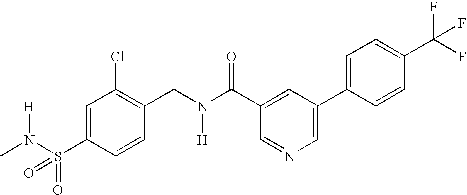 | 483.9 | 484 |
| 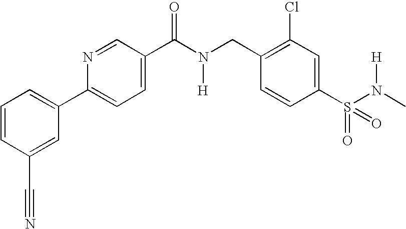 | 440.9 | 441 |
| 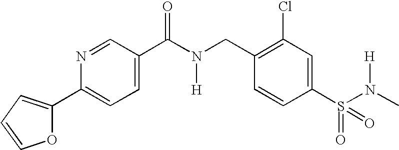 | 405.9 | 406 |
| 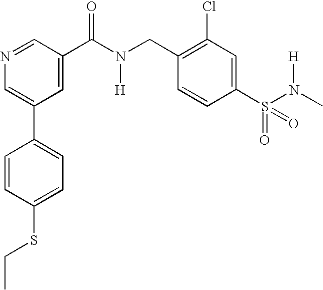 | 476.0 | 477 |
| 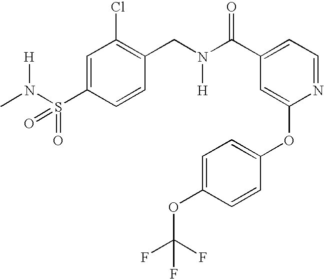 | 515.9 | 516 |
| 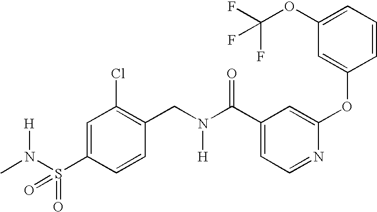 | 515.9 | 516 |
| 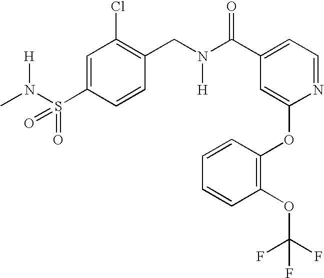 | 515.9 | 516 |
| 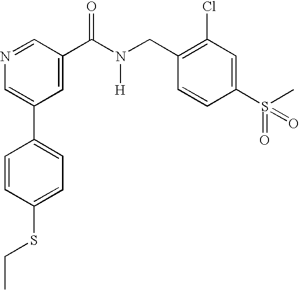 | 461.0 | 461 |
| 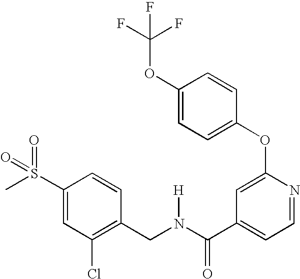 | 500.9 | 501 |
| 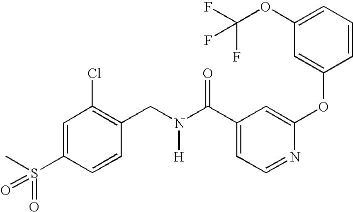 | 500.9 | 501 |
| 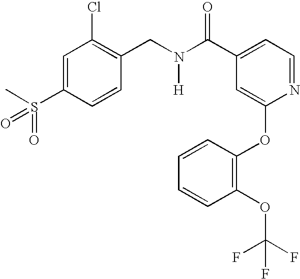 | 500.9 | 501 |
| 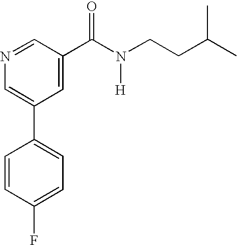 | 286.3 | 287 |
| 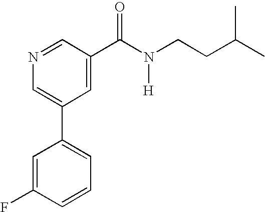 | 286.3 | 287 |
| 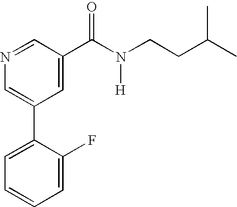 | 286.3 | 287 |
| 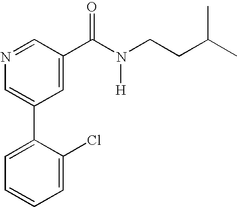 | 302.8 | 303 |
| 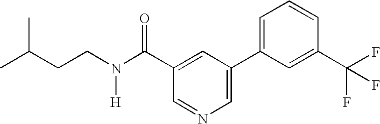 | 336.4 | 337 |
| 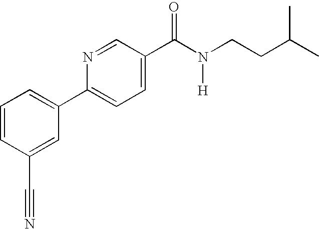 | 293.4 | 294 |
| 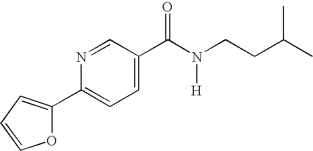 | 258.3 | 259 |
| 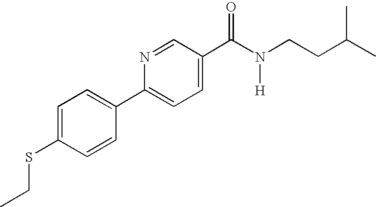 | 328.5 | 329 |
| 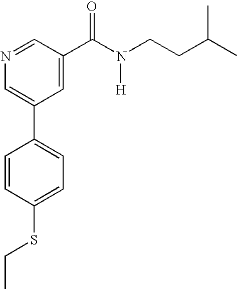 | 328.5 | 329 |
| 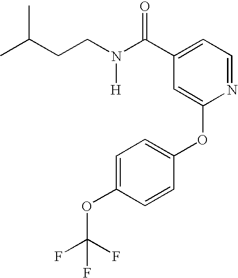 | 368.4 | 369 |
| 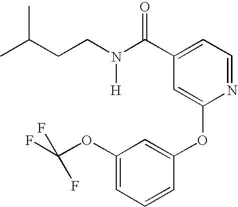 | 368.4 | 369 |
| 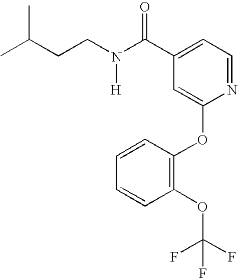 | 368.4 | 369 |
| 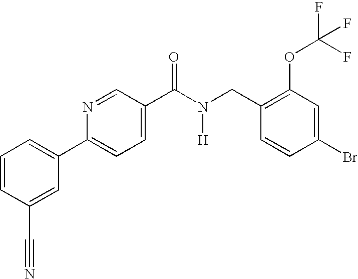 | 476.2 | 477 |
| 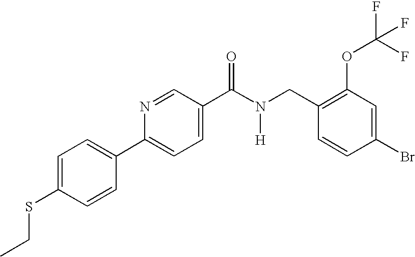 | 511.4 | 512 |
| 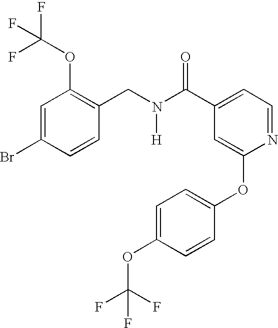 | 551.2 | 552 |
| 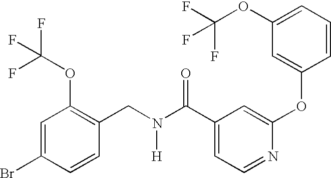 | 551.2 | 552 |
| 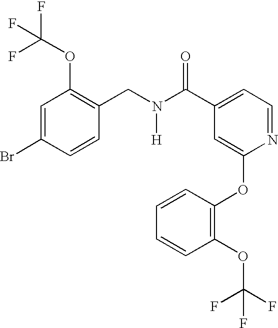 | 551.2 | 552 |
| 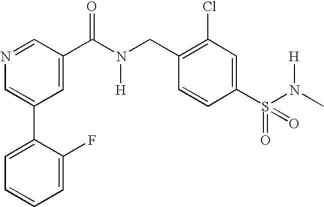 | 433.9 | 434 |
| 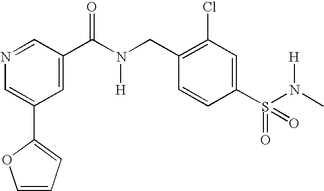 | 405.9 | 406 |
| 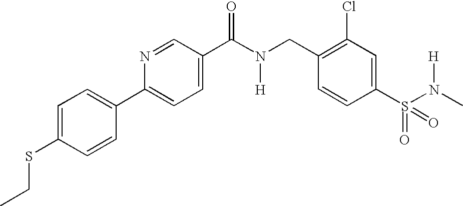 | 476.0 | 477 |
| 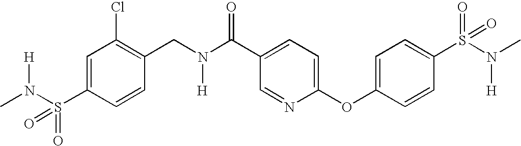 | 525.0 | 525 |
| 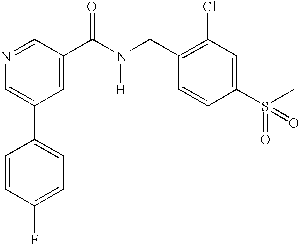 | 418.9 | 419 |
| 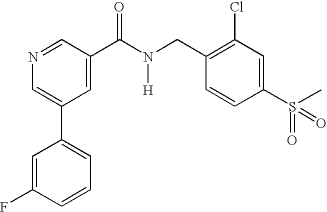 | 418.9 | 419 |
| 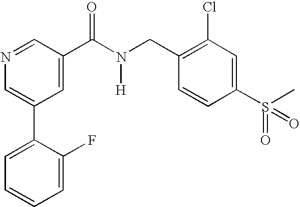 | 418.9 | 419 |
| 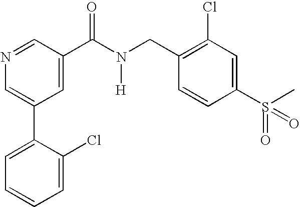 | 435.3 | 436 |
| 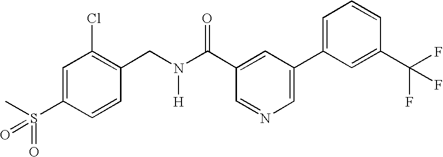 | 468.9 | 469 |
| 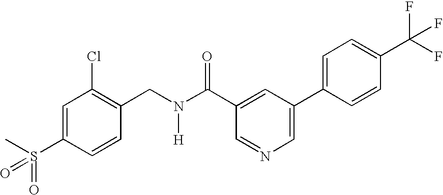 | 468.9 | 469 |
| 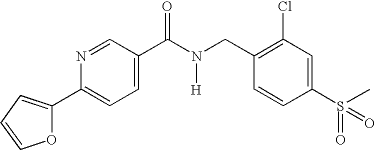 | 390.8 | 391 |
| 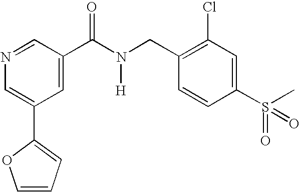 | 390.8 | 391 |
| 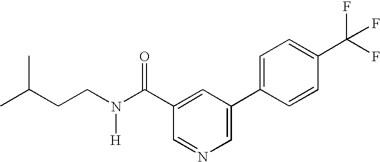 | 336.4 | 337 |
| 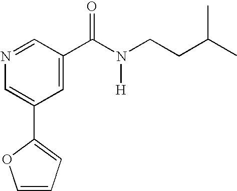 | 258.3 | 259 |
| 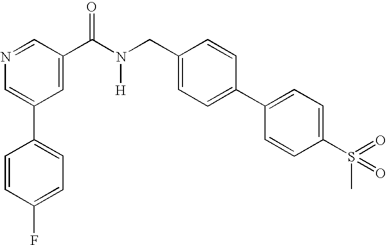 | 460.5 | 461 |
| 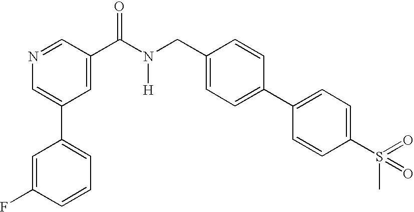 | 460.5 | 461 |
| 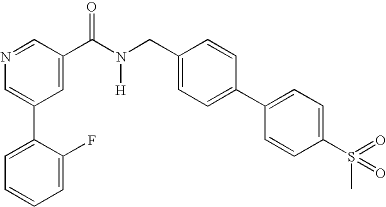 | 460.5 | 461 |
| 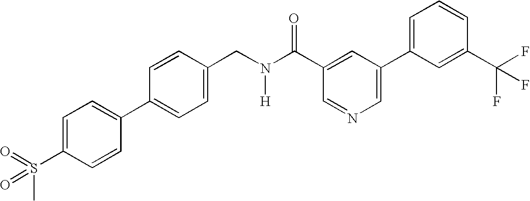 | 510.5 | 511 |
| 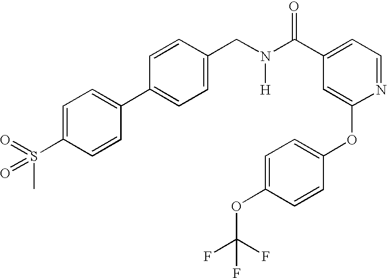 | 542.5 | 543 |
| 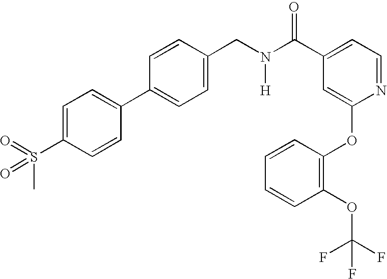 | 542.5 | 543 |
| 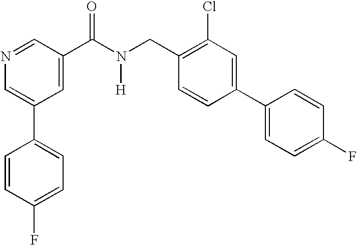 | 434.9 | 435 |
| 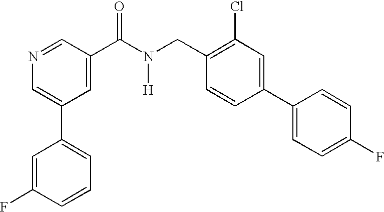 | 434.9 | 435 |
| 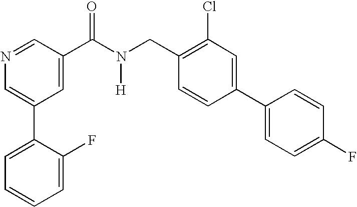 | 434.9 | 435 |
| 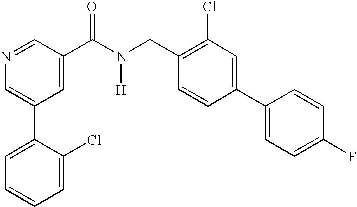 | 451.3 | 452 |
| 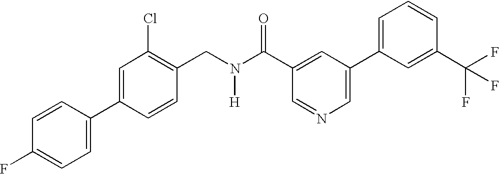 | 484.9 | 485 |
| 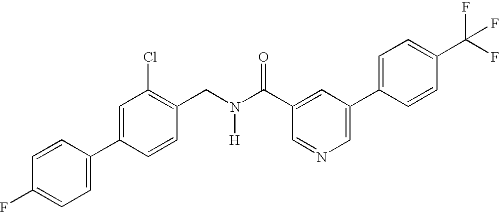 | 484.9 | 485 |
| 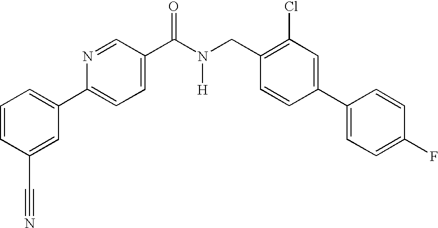 | 441.9 | 442 |
| 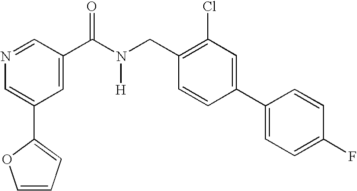 | 406.8 | 407 |
| 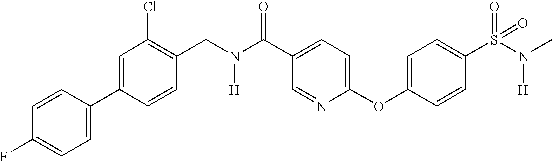 | 526.0 | 526 |
| 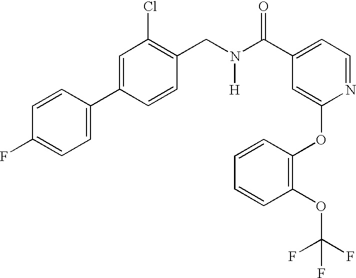 | 516.9 | 517 |
| 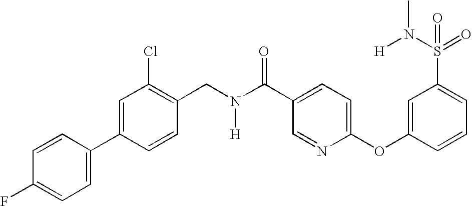 | 526.0 | 526 |
| 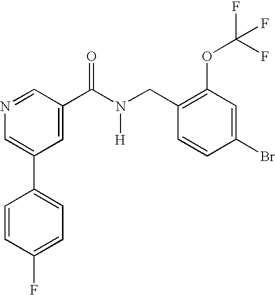 | 469.2 | 470 |
| 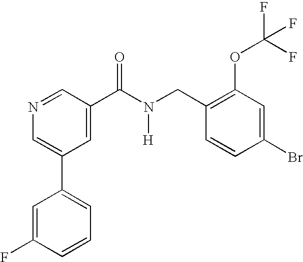 | 469.2 | 470 |
| 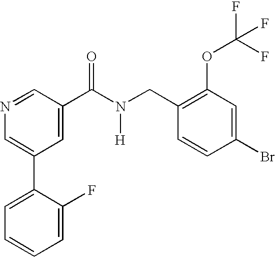 | 469.2 | 470 |
| 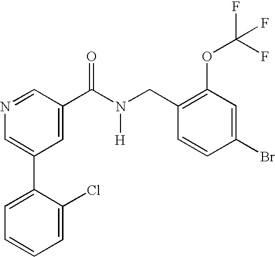 | 485.7 | 486 |
| 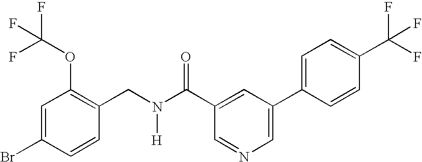 | 519.2 | 520 |
| 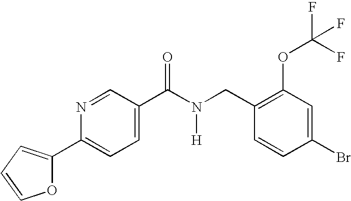 | 441.2 | 442 |
| 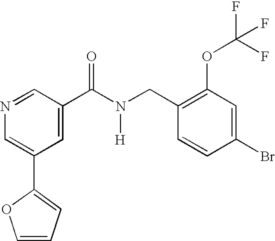 | 441.2 | 442 |
| 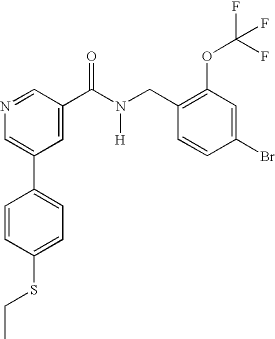 | 511.4 | 512 |
| 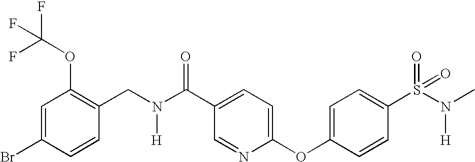 | 560.3 | 561 |
| 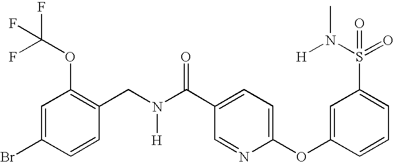 | 560.3 | 561 |

### METHODS OF USE

In accordance with the invention, there are provided methods of using the compounds as described herein and their pharmaceutically acceptable derivatives. The compounds used in the invention prevent the degradation of sEH substrates that have beneficial effects or prevent the formation of metabolites that have adverse effects. The inhibition of sEH is an attractive means for preventing and treating a variety of cardiovascular diseases or conditions e.g., endothelial dysfunction. Thus, the methods of the invention are useful for the treatment of such conditions. These encompass diseases including, but not limited to, type 1 and type 2 diabetes, insulin resistance syndrome, hypertension, atherosclerosis, coronary artery disease, angina, ischemia, ischemic stroke, Raynaud's disease and renal disease.

For therapeutic use, the compounds may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds described herein may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition.

Advantageously, the compounds may then be administered together in a single dosage form. In some embodiments, the pharmaceutical compositions comprising such combinations of compounds contain at least about 5%, but more preferably at least about 20%, of a compound (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the above-described compounds include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

The term “patient” includes both human and non-human mammals.

The term “effective amount” means an amount of a compound according to the invention which, in the context of which it is administered or used, is sufficient to achieve the desired effect or result. Depending on the context, the term effective amount may include or be synonymous with a pharmaceutically effective amount or a diagnostically effective amount.

The terms “pharmaceutically effective amount” or “therapeutically effective amount” means an amount of a compound according to the invention which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the invention which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the invention, and the age, body weight, general health, sex, and diet of the patient. Such a therapeutically effective amount can be determined routinely by one of ordinary skill in the art having regard to their own knowledge, the prior art, and this disclosure.

The term “diagnostically effective amount” means an amount of a compound according to the invention which, when used in a diagnostic method, apparatus, or assay, is sufficient to achieve the desired diagnostic effect or the desired biological activity necessary for the diagnostic method, apparatus, or assay. Such an amount would be sufficient to elicit the biological or medical response in a diagnostic method, apparatus, or assay, which may include a biological or medical response in a patient or in a in vitro or in vivo tissue or system, that is sought by a researcher or clinician. The amount of a compound according to the invention which constitutes a diagnostically effective amount will vary depending on such factors as the compound and its biological activity, the diagnostic method, apparatus, or assay used, the composition used for administration, the time of administration, the route of administration, the rate of excretion of the compound, the duration of administration, drugs and other compounds used in combination with or coincidentally with the compounds of the invention, and, if a patient is the subject of the diagnostic administration, the age, body weight, general health, sex, and diet of the patient. Such a diagnostically effective amount can be determined routinely by one of ordinary skill in the art having regard to their own knowledge, the prior art, and this disclosure.

The terms “treating” or “treatment” mean the treatment of a disease-state in a patient, and include:
(i) preventing the disease-state from occurring in a patient, in particular, when such patient is genetically or otherwise predisposed to the disease-state but has not yet been diagnosed as having it;
(ii) inhibiting or ameliorating the disease-state in a patient, i.e., arresting or slowing its development; or
(iii) relieving the disease-state in a patient, i.e., causing regression or cure of the disease-state.

### In Vitro Assay for Inhibition of hsEH

This high throughput screen identifies compounds that inhibit the interaction of human soluble epoxide hydrolase (sEH) with a tetramethyl rhodamine (TAMRA)-labeled probe. The UHTS employs the Zymark Allegro modular robotic system to dispense reagents, buffers, and test compounds into either 96-well or 384-well black microtiter plates (from Costar). The assay buffer is: 20 mM TES, 200 mM NaCl, 0.05% w/v CHAPS, 1 mM TCEP, pH=7.0. Test compounds dissolved in neat DMSO at 5 mg/mL are diluted to 0.5 mg/mL in neat DMSO. The 0.5 mg/mL solutions are further diluted to 30 μg/mL in assay buffer containing DMSO such that the final concentration of DMSO is 30%. For 384-well format, a mixture of 10.35 nM human sEH and 2.59 nM probe is prepared in assay buffer and 60 μL is added to each well for a final sEH concentration of 10 nM and a final probe concentration of 2.5 nM. 2.1 μL of diluted test compound is then added to each well, where the final assay concentration will be 1 μg/mL test compound and 1% DMSO. The final volume in each well is 62.1 μL. Positive controls are reaction mixtures containing no test compound; negative controls (blanks) are reaction mixtures containing 3 μM BI00611349XX. For 96-well format, the final concentration of all reaction components remains the same. 135 μL sEH/probe mixture is added to wells containing 15 μL test compound so that the final well volume is 150 mL. After incubating the reaction for 30 minutes at room temperature, the plates are read for fluorescence polarization in the LJL Analyst set to 530 nm excitation, 580 nm emission, using the Rh 561 dichroic mirror.

### In Vitro Assay for Inhibition of rsEH

This screen identifies compounds that inhibit the interaction of rat soluble epoxide hydrolase (sEH) with a tetramethyl rhodamine (TAMRA)-labeled probe. The assay employs a Multimek, a Multidrop, and manual multi-channel pipettors to dispense reagents, buffers, and test compounds into 96-well black microtiter plates (Costar 3792). The assay buffer is: 20 mM TES, 200 mM NaCl, 0.05% w/v CHAPS, 1 mM TCEP, pH=7.0. Test compounds dissolved in neat DMSO at 10 mM are diluted to 1.5 mM in neat DMSO. The 1.5 mM solutions are serially diluted using 3-fold dilutions in neat DMSO in polypropylene plates. Assay buffer is added to the wells such that the compounds are diluted 10-fold and the DMSO concentration is 10%. A mixture of 11.1 nM rat sEH and 2.78 nM probe is prepared in assay buffer. 15 uL of diluted test compound is added to each well, where the final maximum assay concentration will be 3 uM test compound and 1% DMSO. 135 uL of sEH/probe mixture is added to each well for a final sEH concentration of 10 nM and a final probe concentration of 2.5 nM. The final volume in each well is 150 uL. Positive controls are reaction mixtures containing no test compound; negative controls (blanks) are reaction mixtures containing 3 uM BI00611349XX. After incubating the reaction for 30 minutes at room temperature, the plates are read for fluorescence polarization in the LJL Analyst set to 530 mm excitation, 580 nm emission, using the Rh 561 dichroic mirror.

1: A compound of the formula (I): 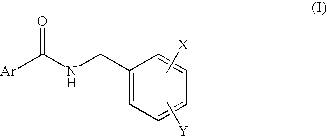
wherein:
Ar is phenyl or pyridinyl each optionally substituted with one to three substituent groups chosen from C₁₋₄ alkyl, C₃₋₆ cycloalkyl, C₂₋₄ alkynyl, C₁₋₄ alkyloxycarbonyl, C₁₋₄ alkylamidocarbonyl, C₁₋₄ dialkylamidocarbonyl, C₁₋₄ alkoxy, C₃₋₆ cycloalkylamino, di(C₃₋₆ cyclo)alkylamino, C₁₋₄ alkylsulfonyl, C₁₋₄ alkylheterocyclyl, halogen, hydroxyl, phenyl, naphthyl, heterocyclyl and heteroaryl
wherein each substituent group is optionally independently substituted with one to three substituents chosen from amino, cyano, carboxy, carboxamido, halogen, hydroxyl, sulfonyl, sulfonamide, C₁₋₄ alkyl, C₃₋₆ cycloalkyl, C₂₋₄ alkynyl, C₁₋₄ alkyloxycarbonyl, C₁₋₄ alkylamidocarbonyl, C₁₋₄ dialkylamidocarbonyl, C₁₋₄ alkoxy, C₃₋₆ cycloalkylamino, di(C₃₋₆ cyclo)alkylamino, C₁₋₄ alkylsulfonyl, C₁₋₄ alkylheterocyclyl, phenyl or pyridinyl optionally substituted with one to three substituents chosen from amino, cyano, halogen, hydroxyl and C₁₋₄ alkylsulfonyl, C₁₋₆ alkyl optionally substituted with hydroxyl, amino, C₁₋₄ alkoxy, C₁₋₄ alkylamino, C₁₋₄ alkylthio, or one to three fluorine atoms;
X and Y are optionally independently hydrogen, halogen, cyano, NH—R, OR, R, SO₂R or S(O)₂NRR, wherein R is independently hydrogen, aryl or C₁₋₆ alkyl optionally substituted with hydroxyl, amino, C₁₋₄ alkoxy, C₁₋₄ alkylamino, C₁₋₄ alkylthio, or one to three fluorine atoms;
and wherein both X and Y are not hydrogen,
or the pharmaceutically acceptable salts thereof.

2: The compound according to claim 1 and wherein:
Ar is pyridinyl optionally substituted with one to three substituent groups chosen from C₁₋₄ alkyl, C₃₋₆ cycloalkyl, C₂₋₄ alkynyl, C₁₋₄ alkyloxycarbonyl, C₁₋₄ alkylamidocarbonyl, C₁₋₄ dialkylamidocarbonyl, C₁₋₄ alkoxy, C₃₋₆ cycloalkylamino, di(C₃₋₆ cyclo)alkylamino, C₁₋₄ alkylsulfonyl, C₁₋₄ alkylheterocyclyl, halogen, hydroxyl, pyridinyl, pyrazinyl, pyridazinyl, quinolinyl and isoquinolinyl;
wherein each substituent group is optionally independently substituted with one to three substituents chosen from amino, cyano, carboxy, carboxamido, halogen, hydroxyl, sulfonyl, sulfonamide, C₁₋₄ alkylsulfonyl, C₁₋₄ alkylheterocyclyl, phenyl or pyridinyl optionally substituted with one to three substituents chosen from amino, cyano, halogen, hydroxyl and C₁₋₄ alkylsulfonyl, C₁₋₆ alkyl optionally substituted with hydroxyl, amino, C₁₋₄ alkoxy, C₁₋₄ alkylamino, C₁₋₄ alkylthio, or one to three fluorine atoms;
X and Y are optionally independently hydrogen, halogen, cyano, NH—R, OR, R, SO₂R, wherein R is independently hydrogen, aryl optionally substituted with one to three substituents chosen from amino, cyano, halogen, hydroxyl and C₁₋₄ alkylsulfonyl, C₁₋₆ alkyl optionally substituted with hydroxyl, amino, C₁₋₄ alkoxy, C₁₋₄ alkylamino, C₁₋₄ alkylthio, or one to three fluorine atoms.

3: The compound according to claim 2 and wherein:
Ar is pyridinyl optionally substituted with one to three substituent groups chosen from C₁₋₄ alkyl, C₁₋₄ alkoxy, C₃₋₆ cycloalkylamino, di(C₃₋₆ cyclo)alkylamino, C₁₋₄ alkylsulfonyl, C₁₋₄ alkylheterocyclyl, halogen and hydroxyl,
wherein each substituent group is optionally independently substituted with one to three substituents chosen from amino, cyano, carboxy, carboxamido, halogen, hydroxyl, sulfonyl, sulfonamide, C₁₋₄ alkylsulfonyl, C₁₋₄ alkylheterocyclyl, phenyl or pyridinyl optionally substituted with one to three substituents chosen from amino, cyano, halogen, hydroxyl and C₁₋₄ alkylsulfonyl, C₁₋₆ alkyl optionally substituted with hydroxyl, amino, C₁₋₄ alkoxy, C₁₋₄ alkylamino, C₁₋₄ alkylthio, or one to three fluorine atoms;
X and Y are optionally independently hydrogen, chloro, bromo, cyano, OR, SO₂R, wherein R is independently hydrogen, C₁₋₄ alkyl or aryl optionally substituted with one to three substituents chosen from amino, cyano, halogen, hydroxyl and C₁₋₄ alkylsulfonyl.

4: A compound of the formula (II): 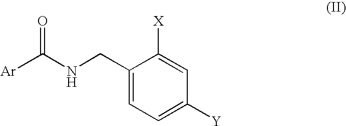
wherein for the Formula (II), the component 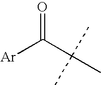
is chosen from A1-A59 in the table I below; in combination with any component 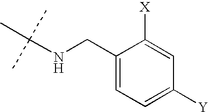
chosen from B1-B9 in the table I below;
TABLE I

| | 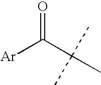 | | 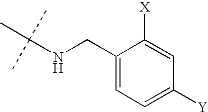 |
|---|---|---|---|
| A1 | 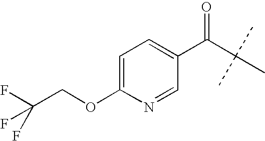 | B1 | 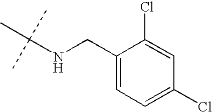 |
| A2 | 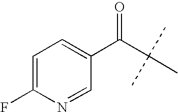 | B2 | 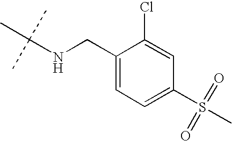 |
| A3 | 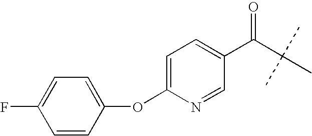 | B3 | 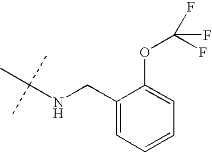 |
| A4 | 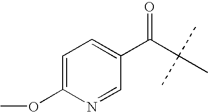 | B4 | 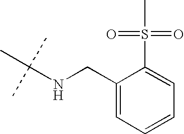 |
| A5 | 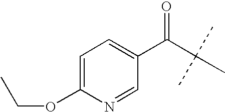 | B5 | 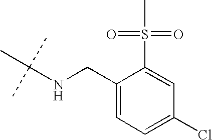 |
| A6 | 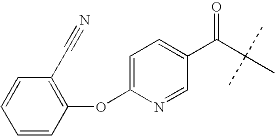 | B6 | 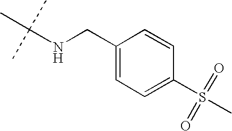 |
| A7 | 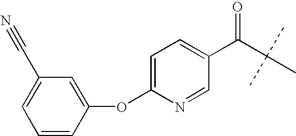 | B7 | 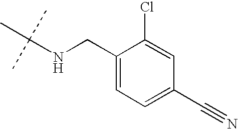 |
| A8 | 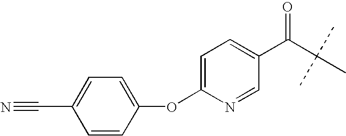 | B8 | 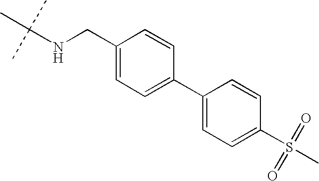 |
| A9 | 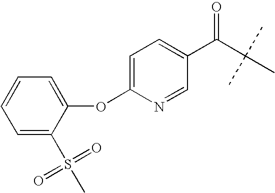 | B9 | 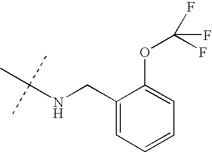 |
| A10 | 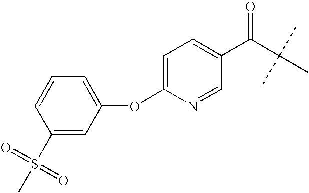 | | |
| A11 | 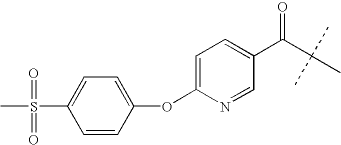 | | |
| A12 | 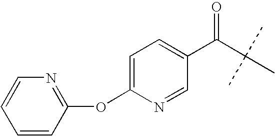 | | |
| A13 | 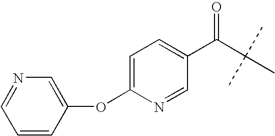 | | |
| A14 | 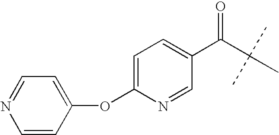 | | |
| A15 | 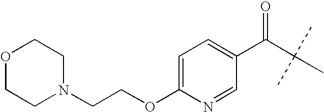 | | |
| A16 | 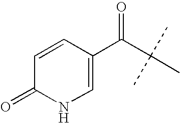 | | |
| A17 | 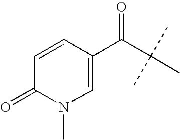 | | |
| A18 | 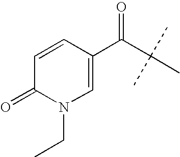 | | |
| A19 | 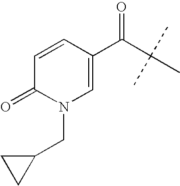 | | |
| A20 | 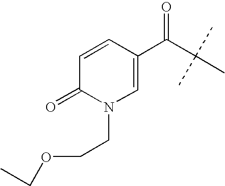 | | |
| A21 | 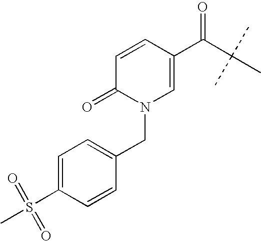 | | |
| A22 | 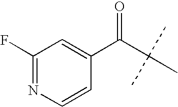 | | |
| A23 | 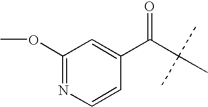 | | |
| A24 | 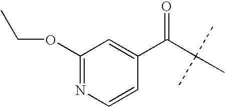 | | |
| A25 | 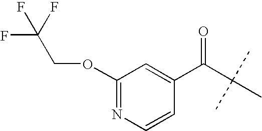 | | |
| A26 | 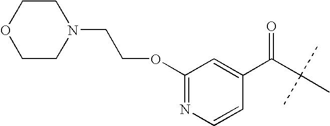 | | |
| A27 | 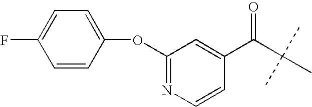 | | |
| A28 | 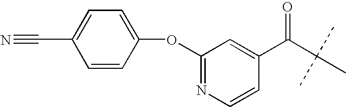 | | |
| A29 | 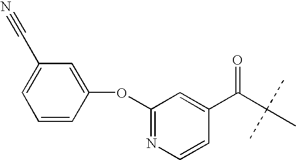 | | |
| A30 | 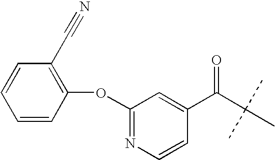 | | |
| A31 | 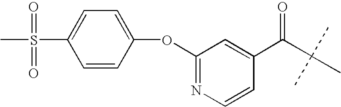 | | |
| A32 | 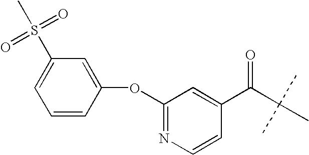 | | |
| A33 | 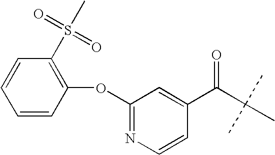 | | |
| A34 | 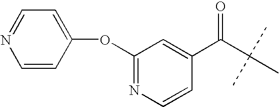 | | |
| A35 | 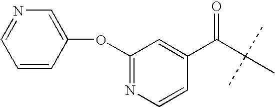 | | |
| A36 | 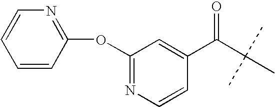 | | |
| A37 | 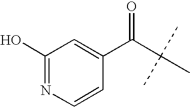 | | |
| A38 | 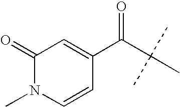 | | |
| A39 | 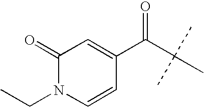 | | |
| A40 | 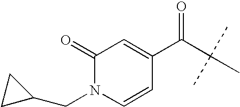 | | |
| A41 | 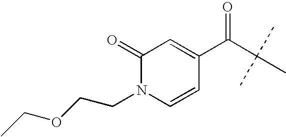 | | |
| A42 | 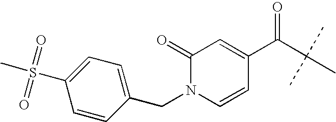 | | |
| A43 | 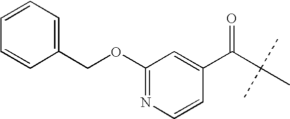 | | |
| A44 | 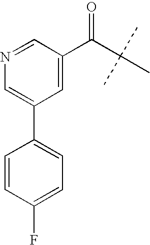 | | |
| A45 | 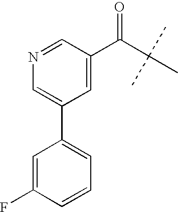 | | |
| A46 | 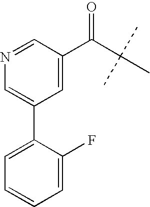 | | |
| A47 | 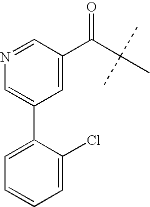 | | |
| A48 | 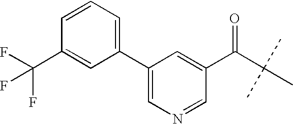 | | |
| A49 | 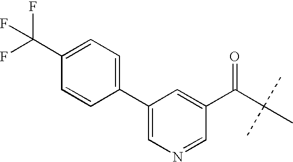 | | |
| A50 | 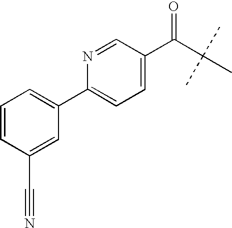 | | |
| A51 | 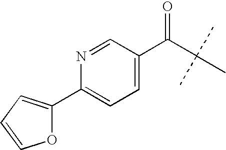 | | |
| A52 | 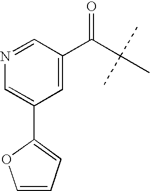 | | |
| A53 | 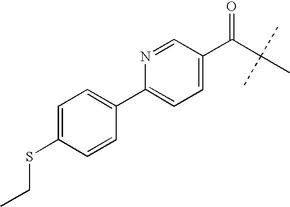 | | |
| A54 | 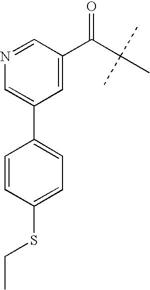 | | |
| A55 | 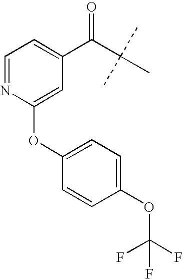 | | |
| A56 | 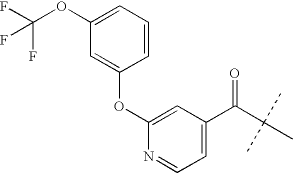 | | |
| A57 | 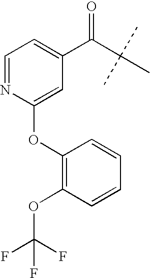 | | |
| A58 | 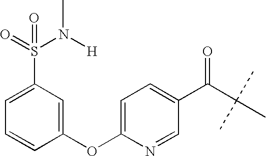 | | |
| A59 | 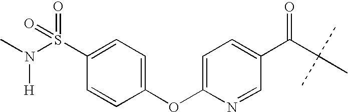 | | |

or the pharmaceutically acceptable salts thereof.

5: A compound of the formula (III): 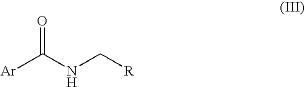
wherein for the Formula (III), the component 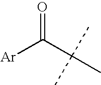
is chosen from A1-A49 in the table IV below; in combination with component B1 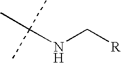
as shown in the table IV below;
TABLE IV

| | 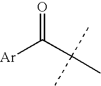 | | 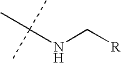 |
|---|---|---|---|
| A1 | 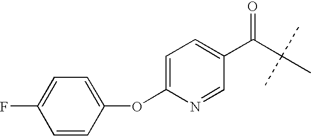 | B1 | 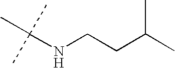 |
| A2 | 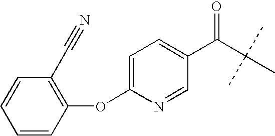 | | |
| A3 | 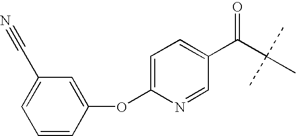 | | |
| A4 | 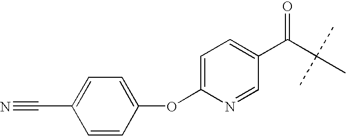 | | |
| A5 | 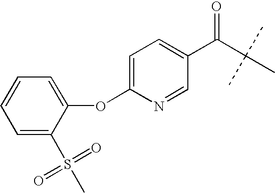 | | |
| A6 | 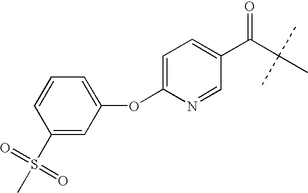 | | |
| A7 | 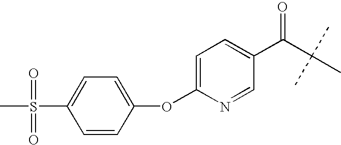 | | |
| A8 | 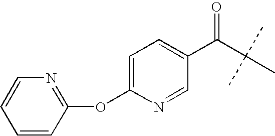 | | |
| A9 | 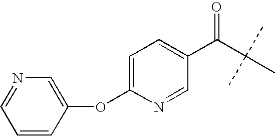 | | |
| A10 | 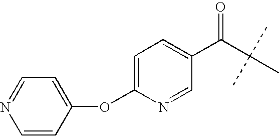 | | |
| A11 | 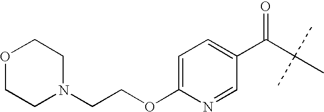 | | |
| A12 | 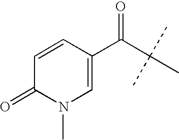 | | |
| A13 | 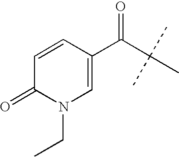 | | |
| A14 | 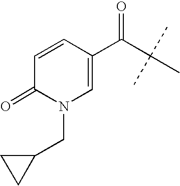 | | |
| A15 | 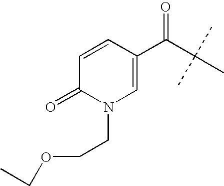 | | |
| A16 | 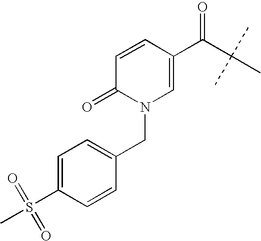 | | |
| A17 | 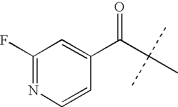 | | |
| A18 | 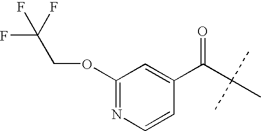 | | |
| A19 | 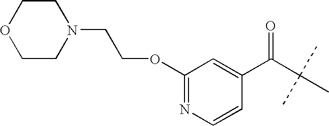 | | |
| A20 | 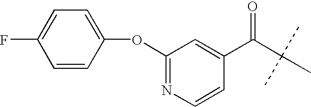 | | |
| A21 | 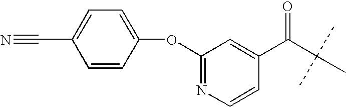 | | |
| A22 | 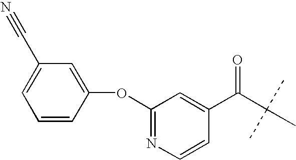 | | |
| A23 | 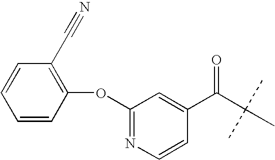 | | |
| A24 | 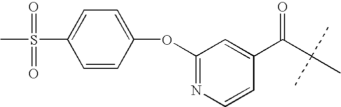 | | |
| A25 | 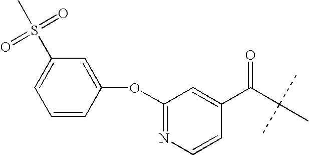 | | |
| A26 | 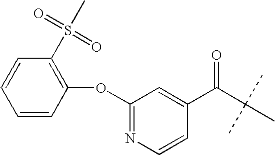 | | |
| A27 | 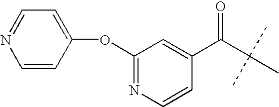 | | |
| A28 | 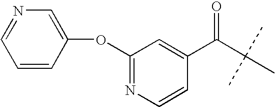 | | |
| A29 | 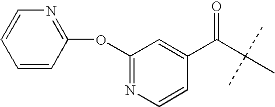 | | |
| A30 | 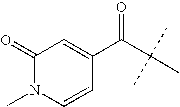 | | |
| A31 | 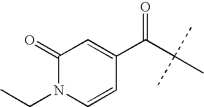 | | |
| A32 | 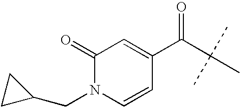 | | |
| A33 | 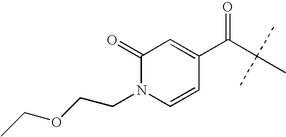 | | |
| A34 | 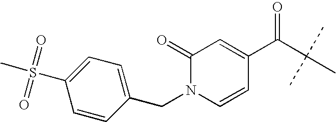 | | |
| A35 | 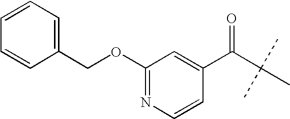 | | |
| A36 | 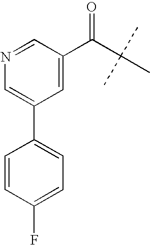 | | |
| A37 | 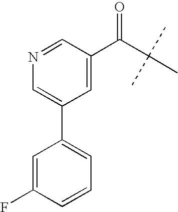 | | |
| A38 | 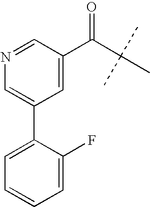 | | |
| A39 | 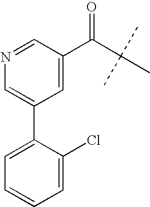 | | |
| A40 | 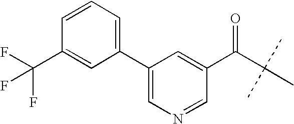 | | |
| A41 | 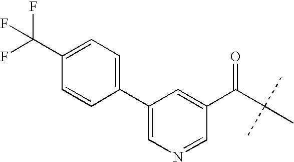 | | |
| A42 | 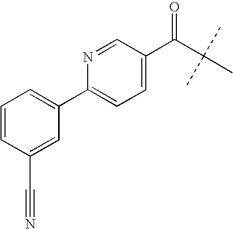 | | |
| A43 | 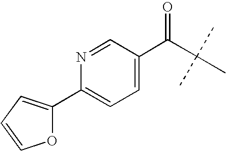 | | |
| A44 | 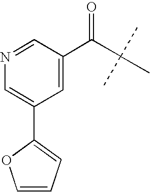 | | |
| A45 | 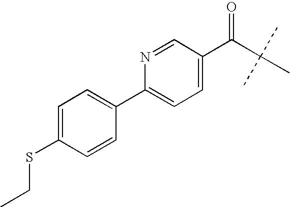 | | |
| A46 | 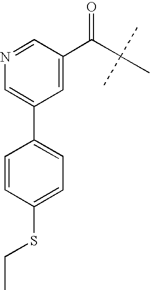 | | |
| A47 | 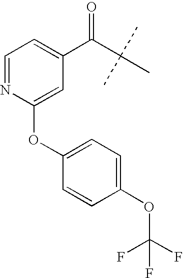 | | |
| A48 | 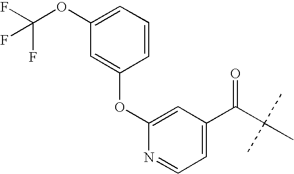 | | |
| A49 | 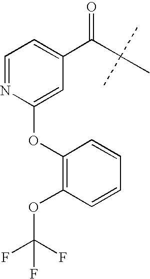 | | |

or the pharmaceutically acceptable salts thereof.

6: A compound chosen from 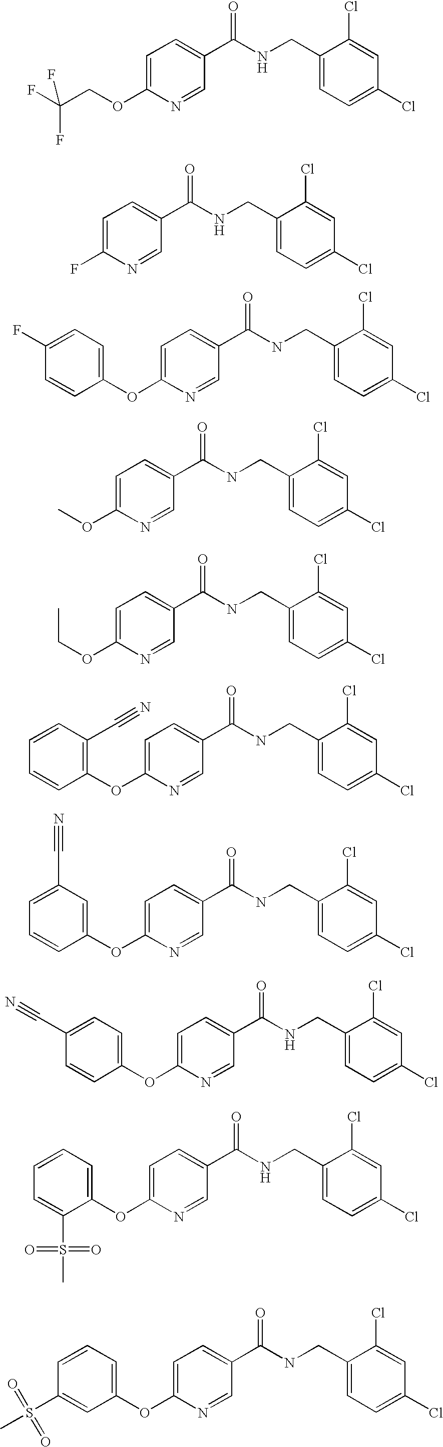 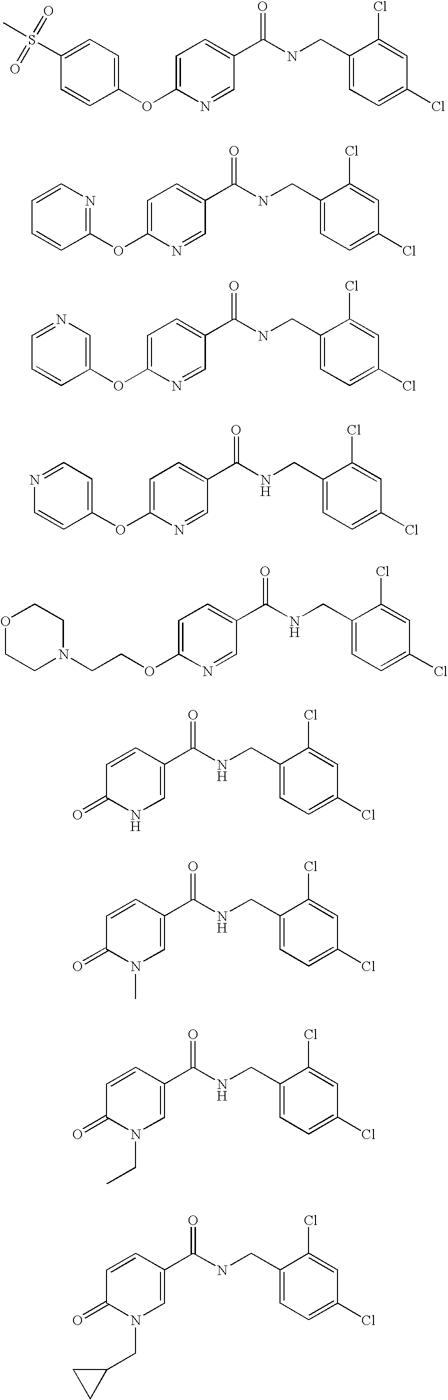 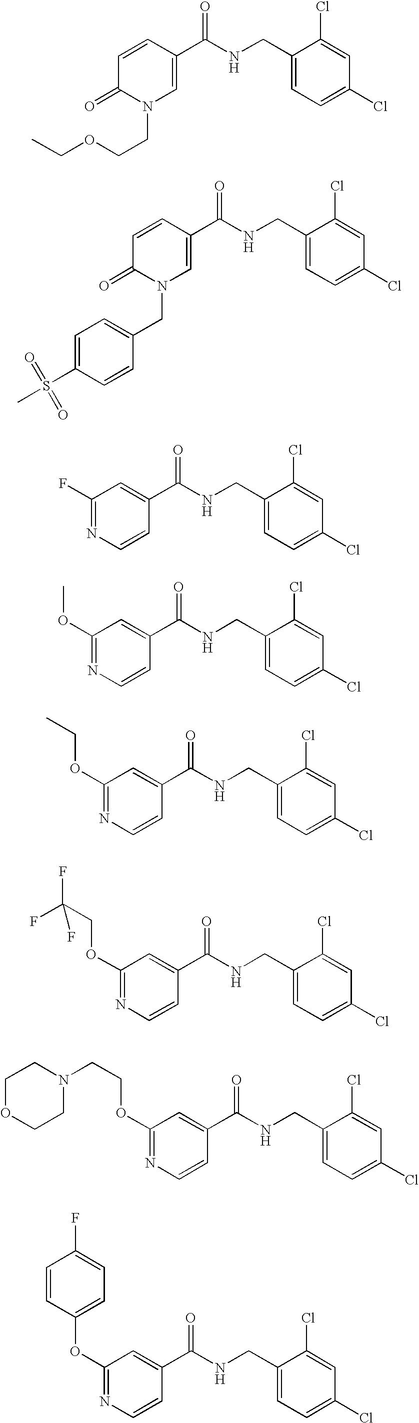 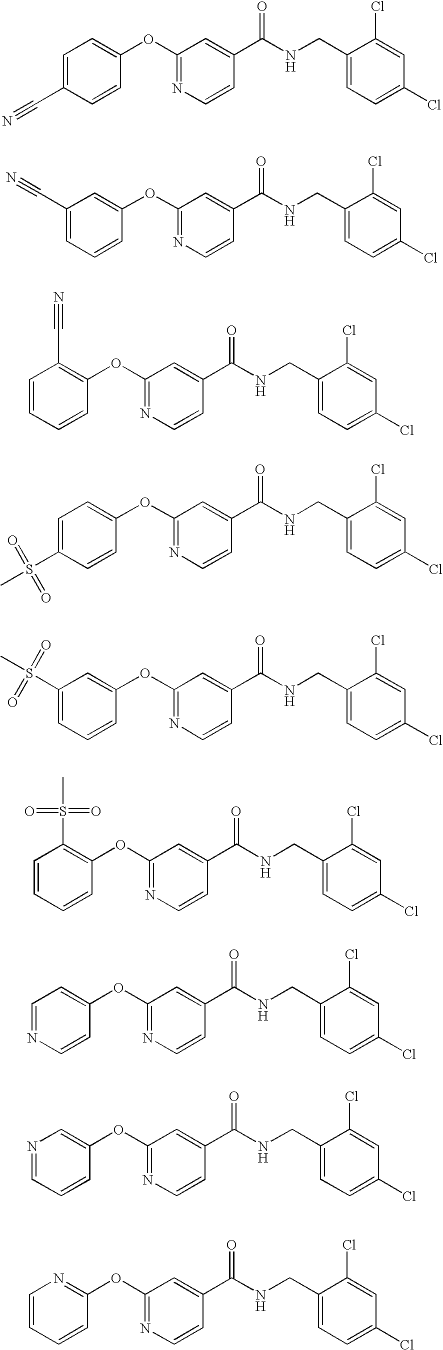 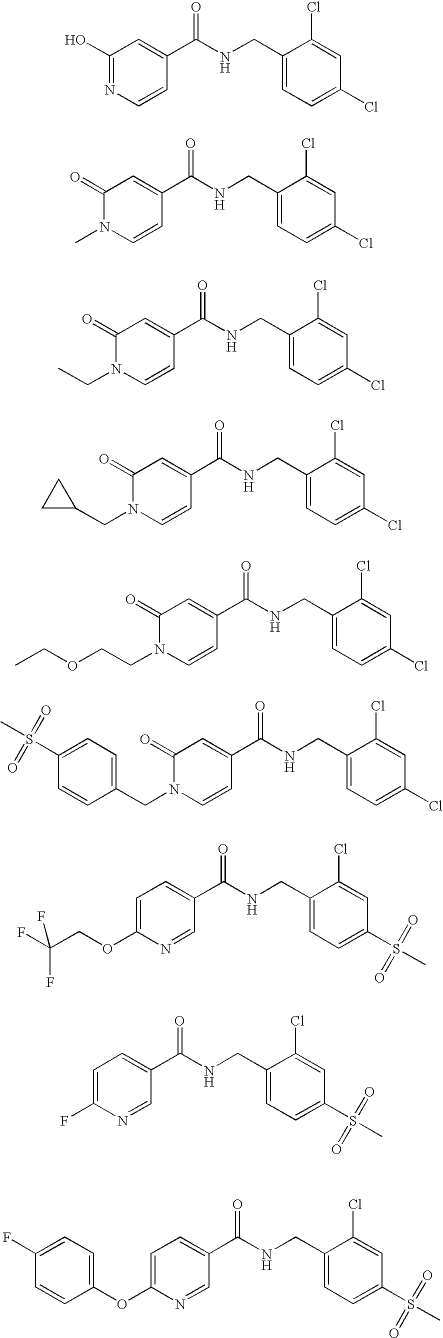 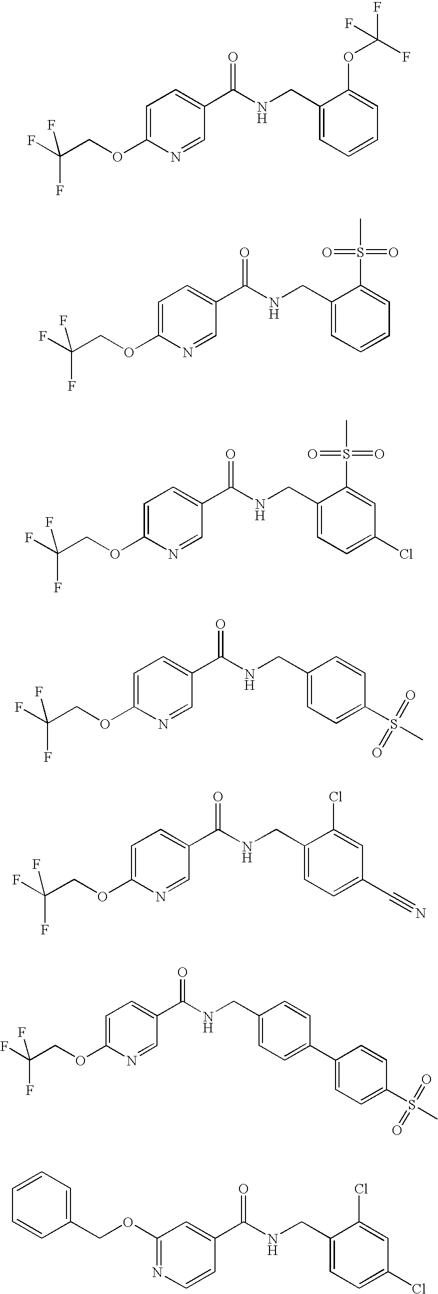 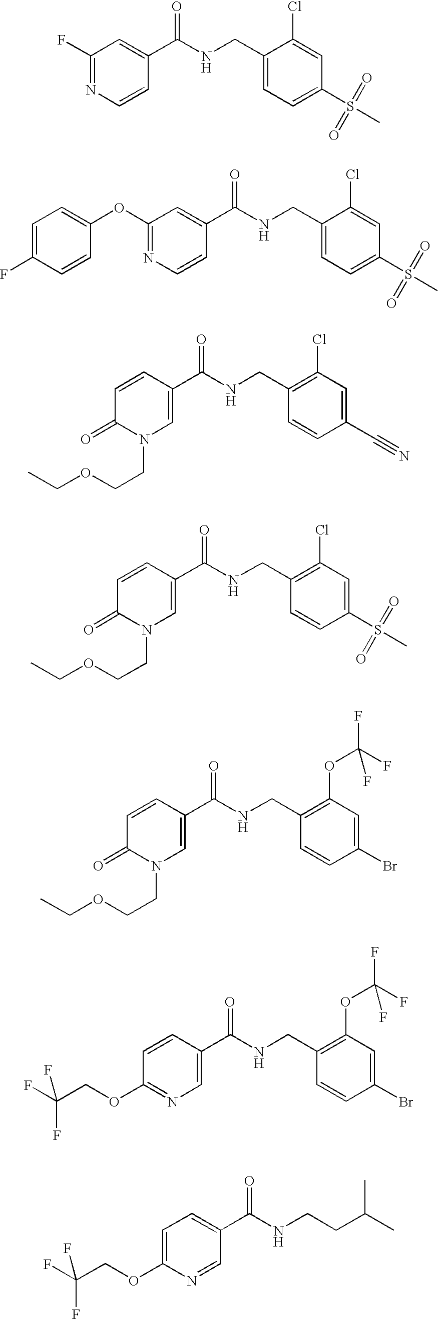
or the pharmaceutically acceptable salts thereof.

7: A compound chosen from

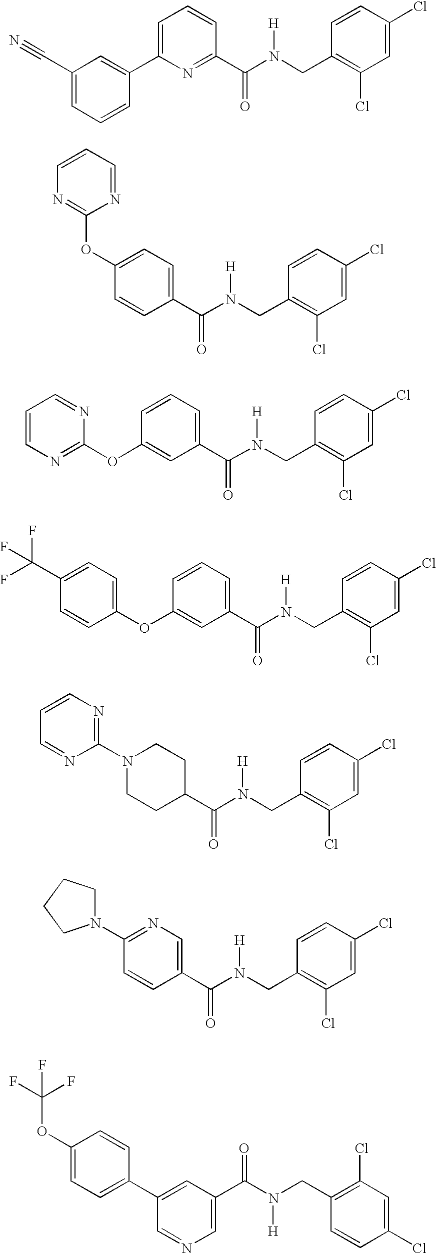

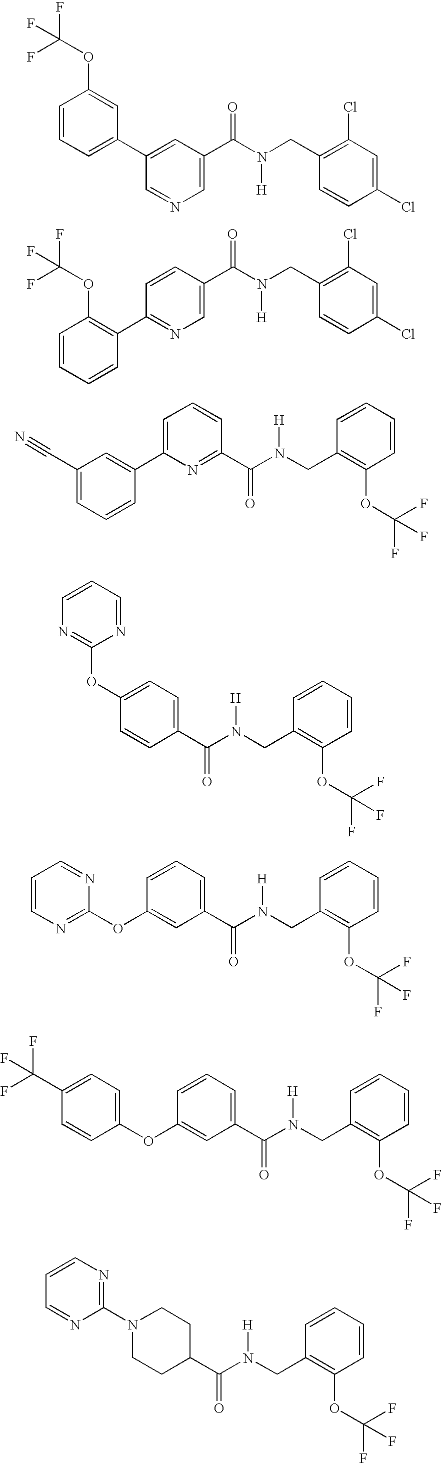

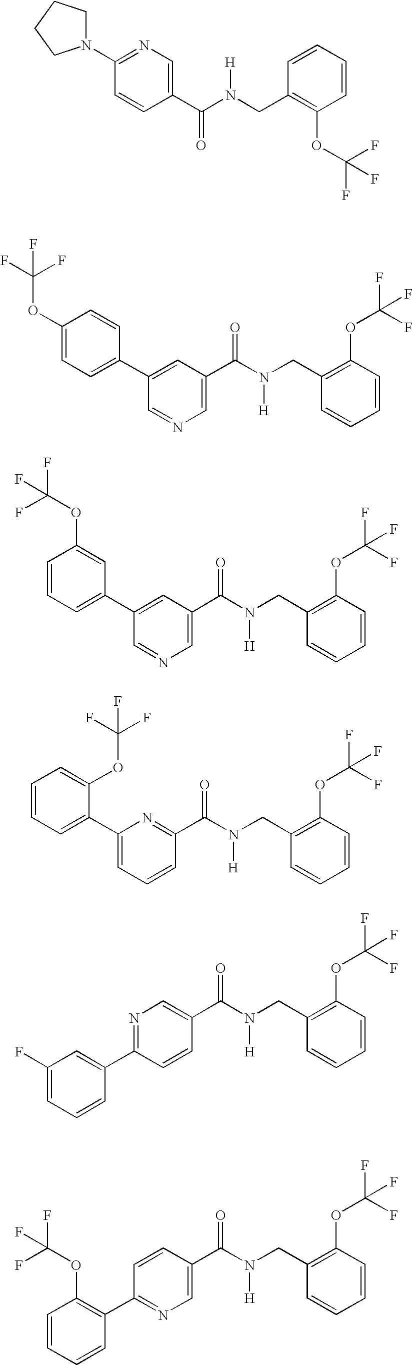

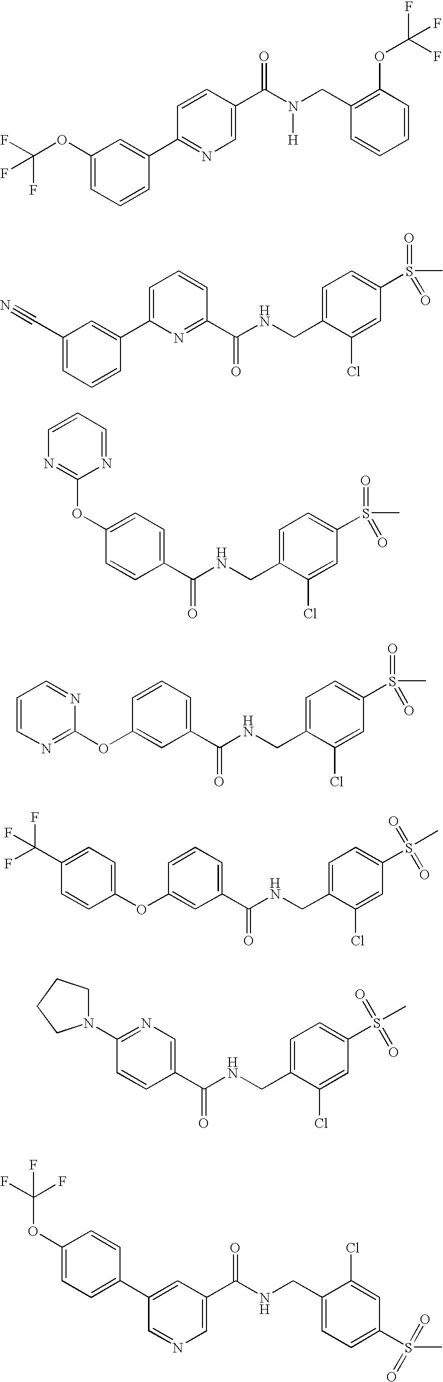

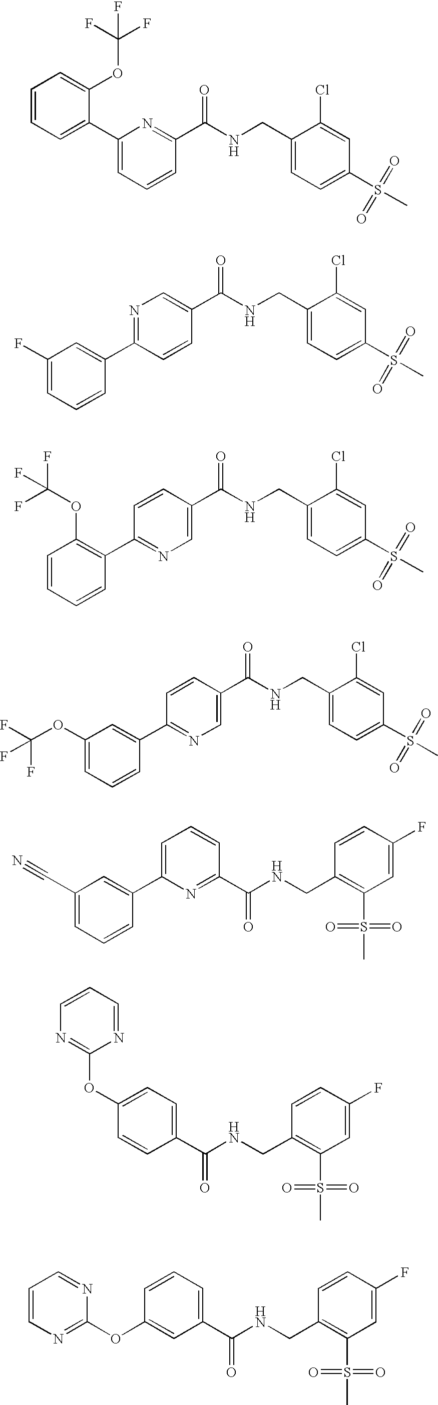

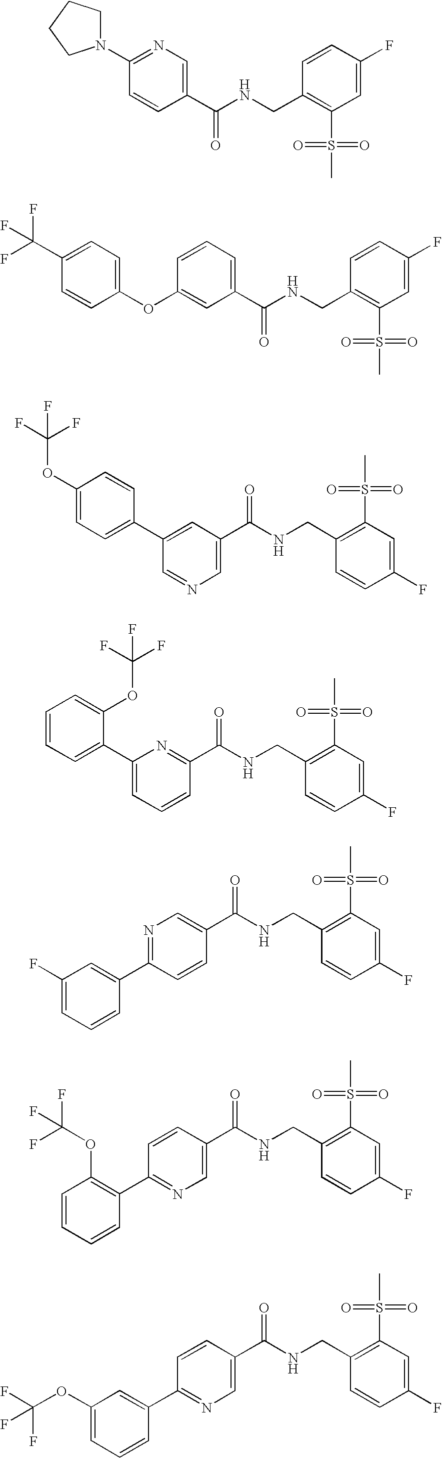

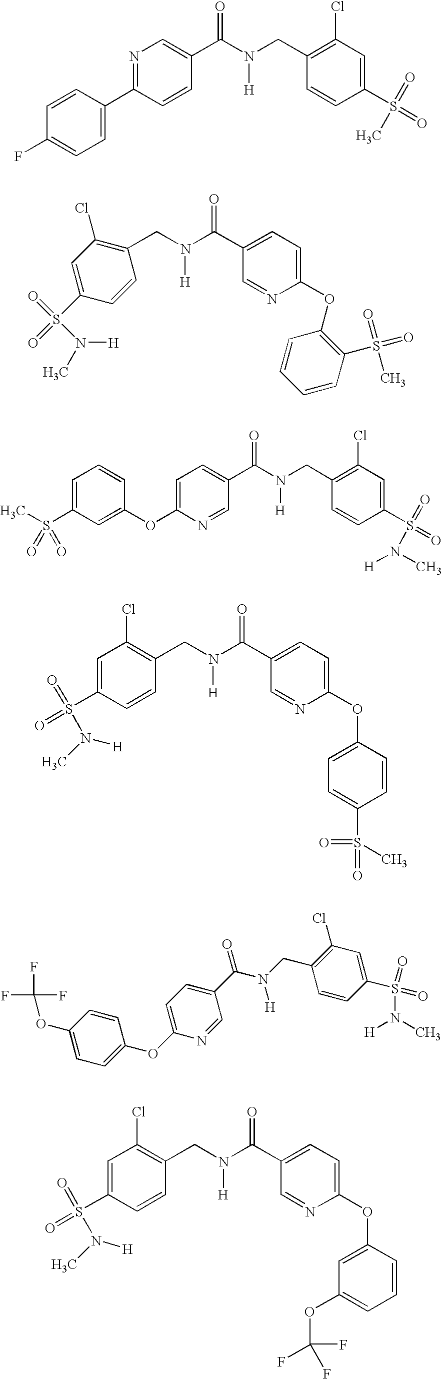

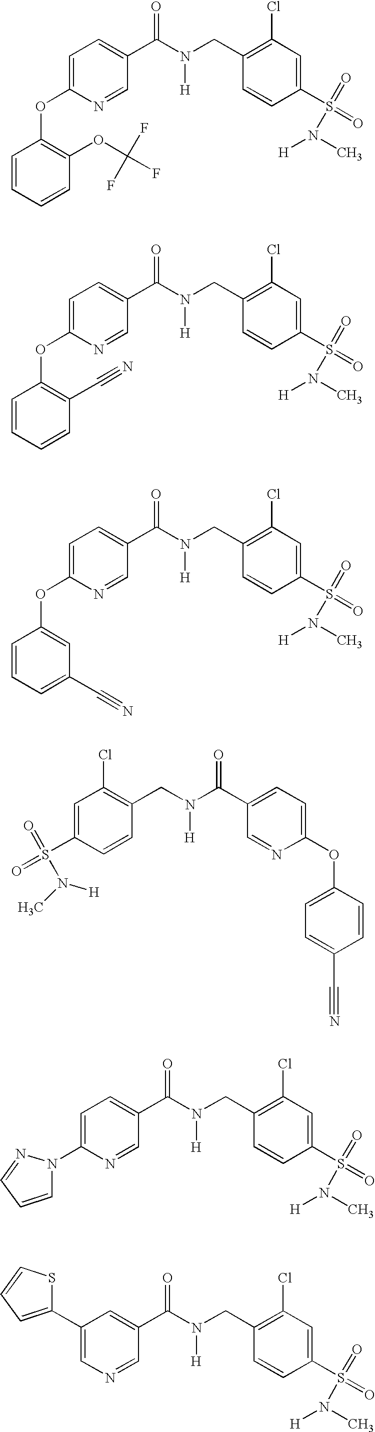

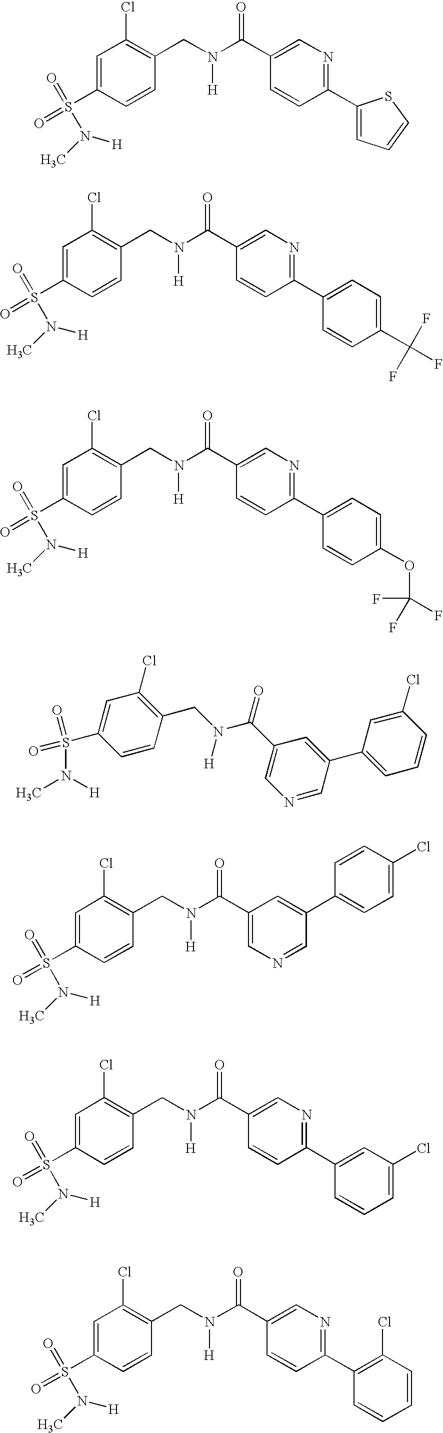

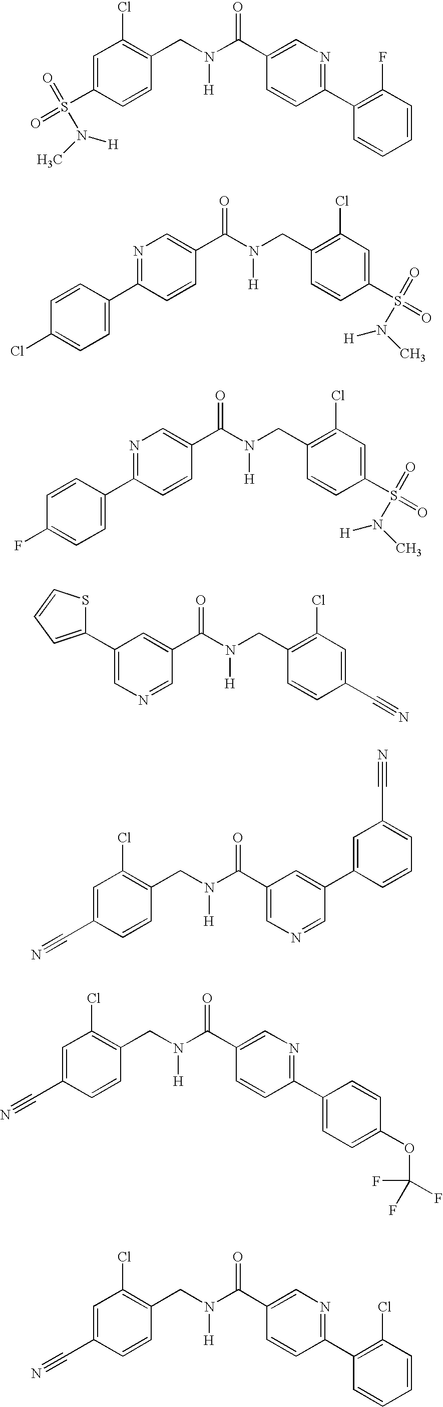

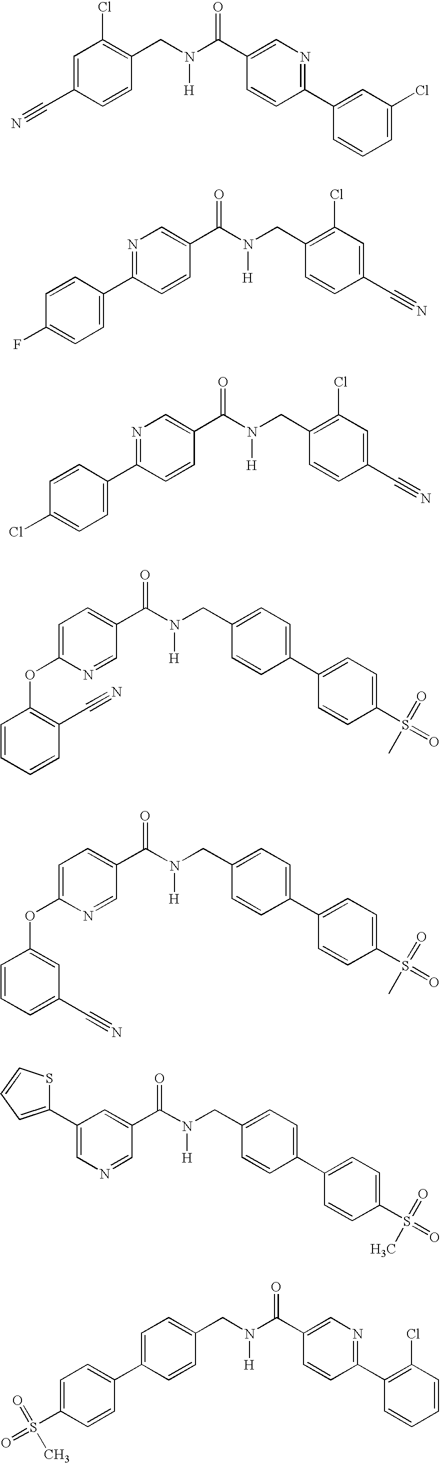

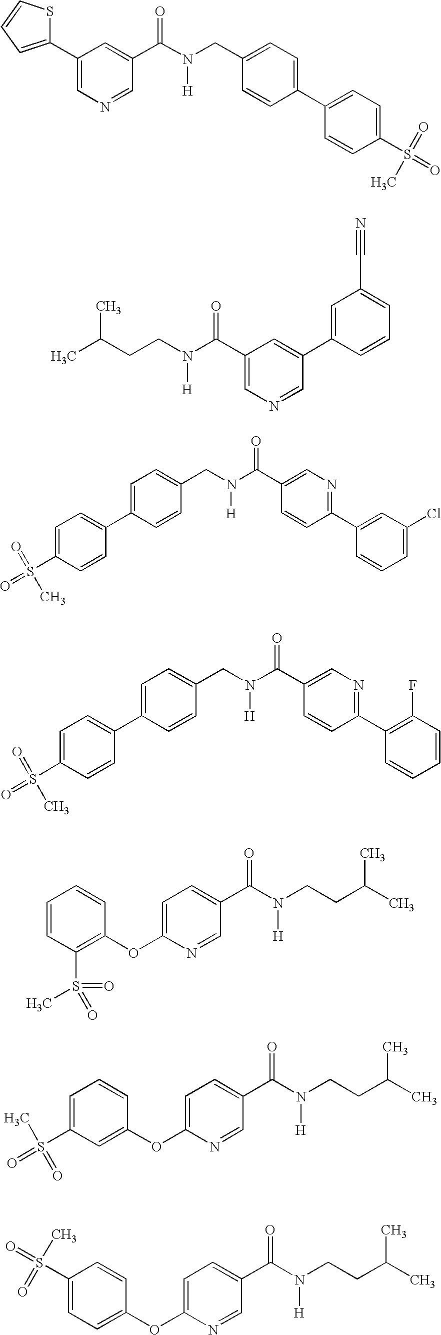

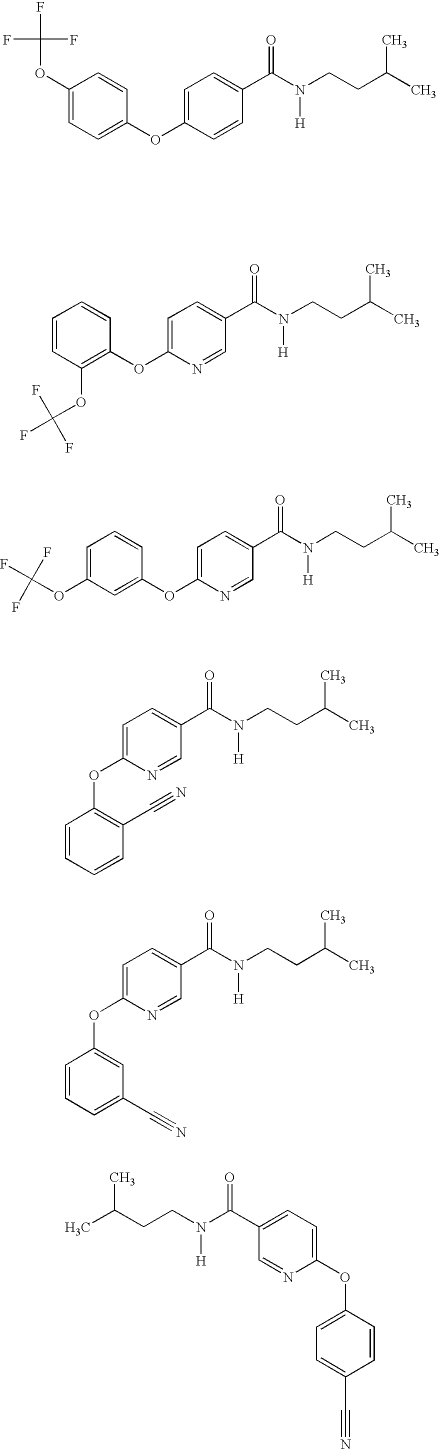

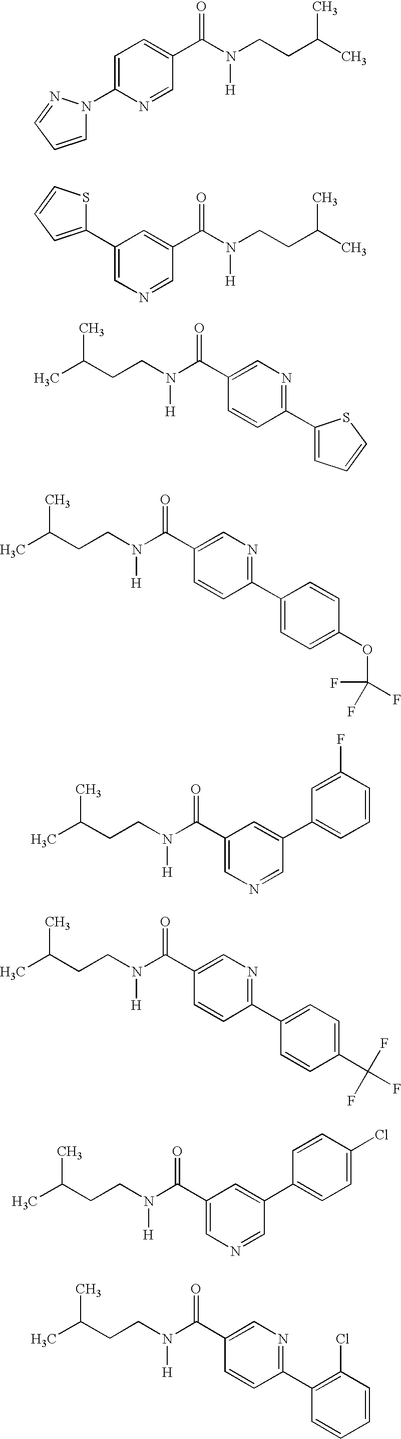

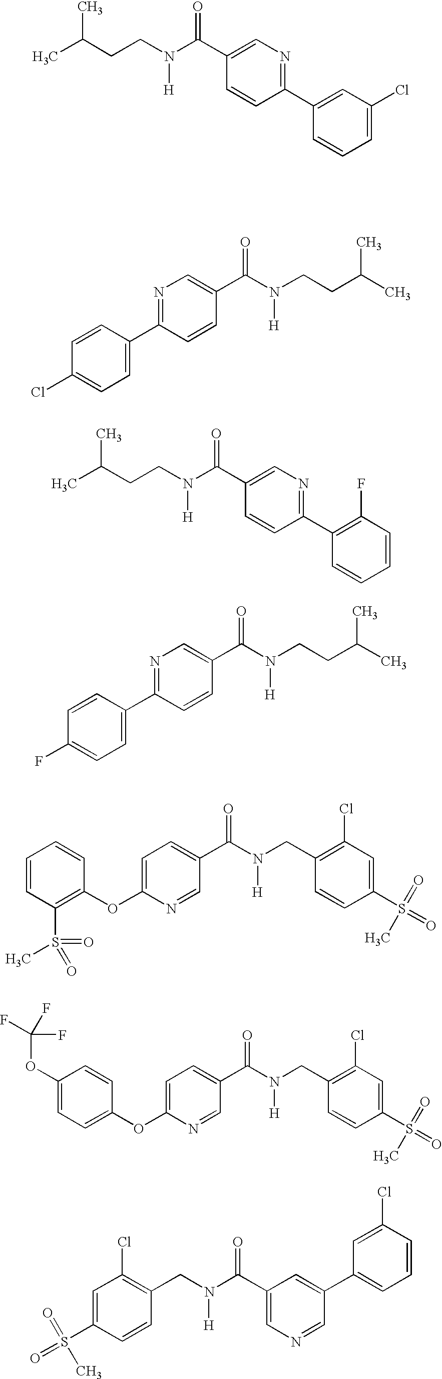

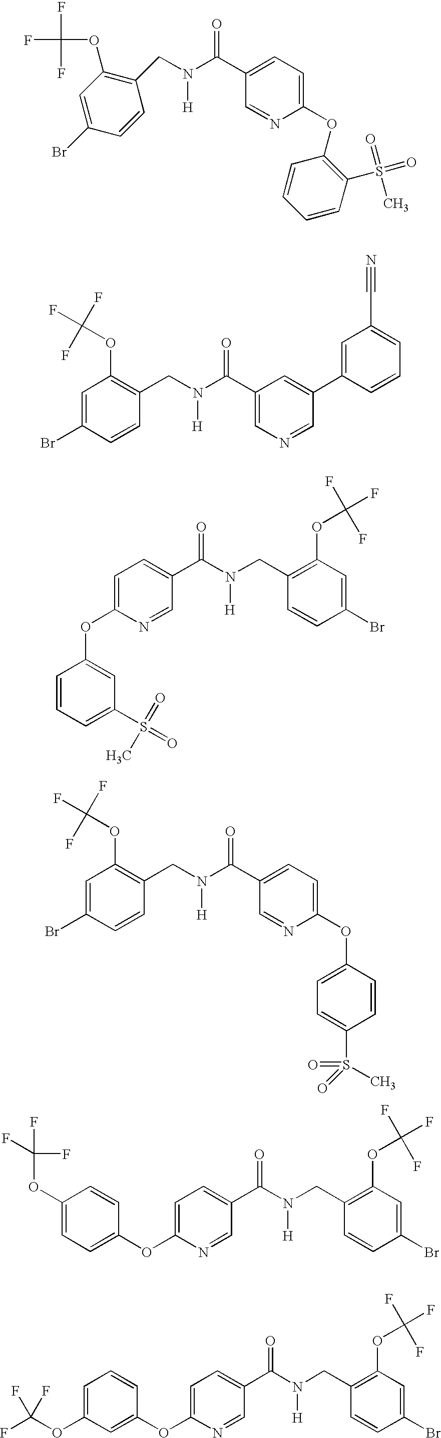

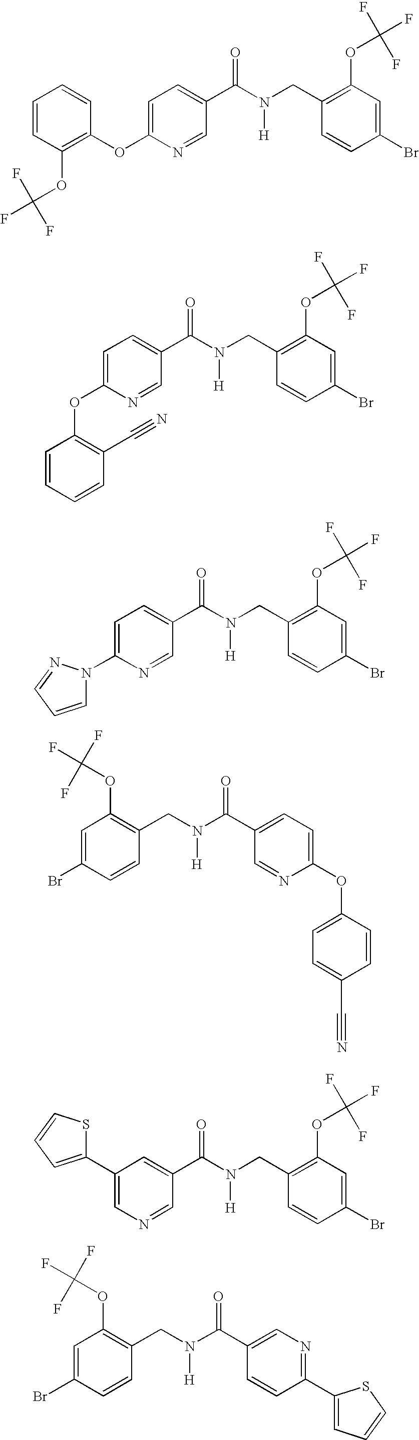

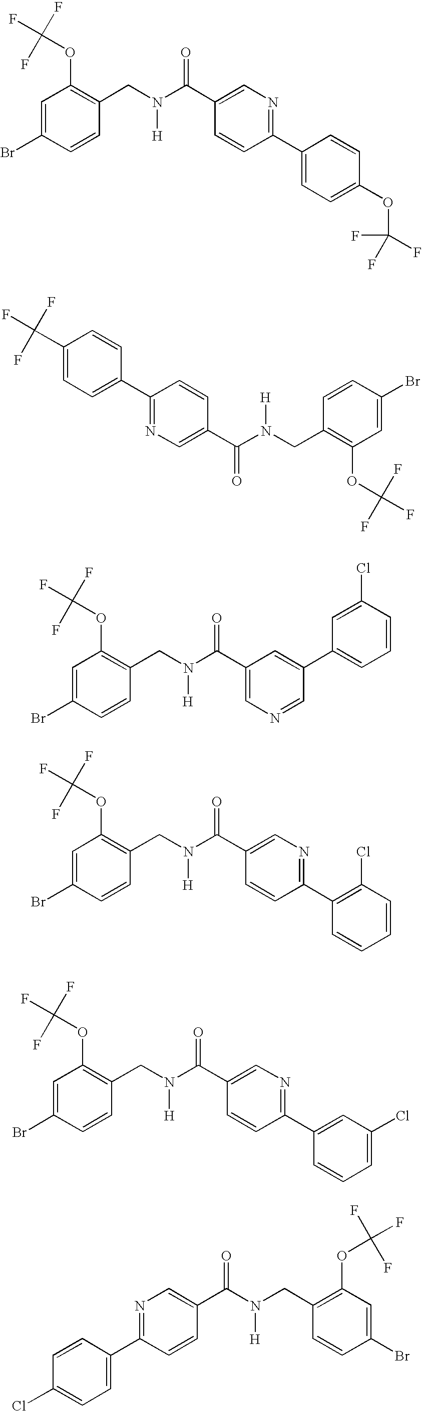

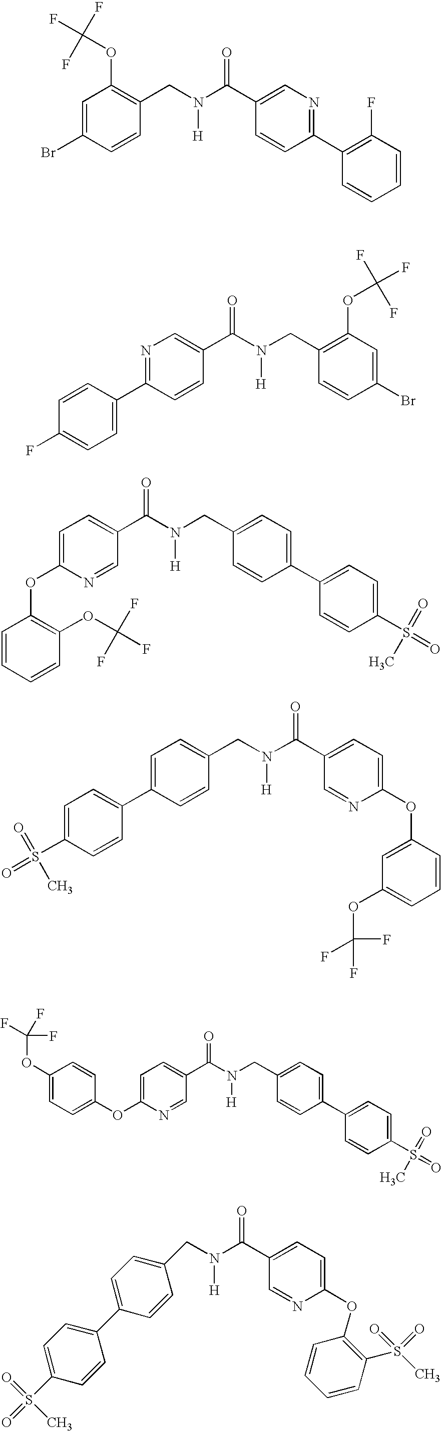

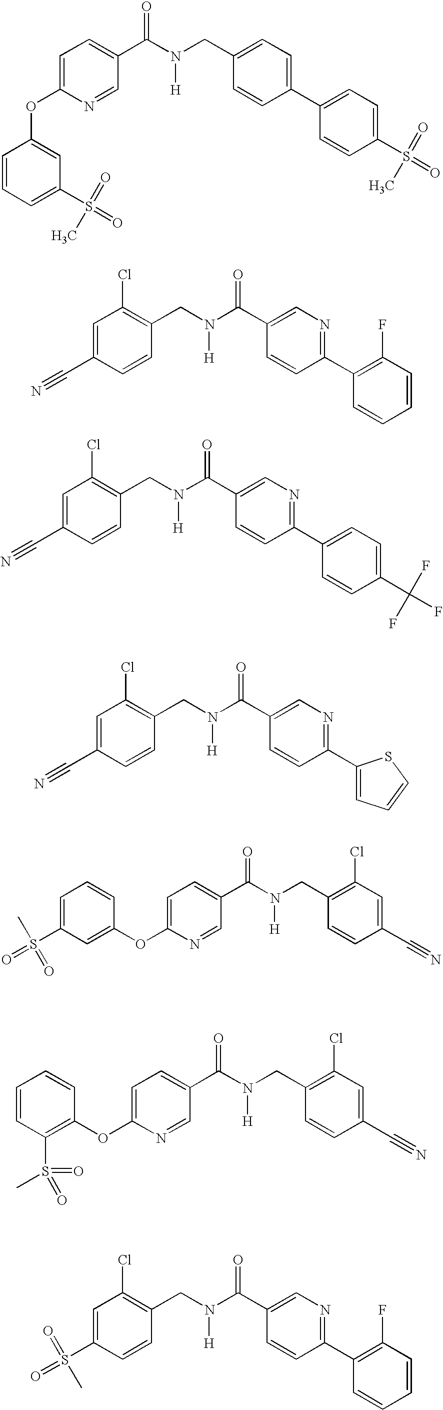

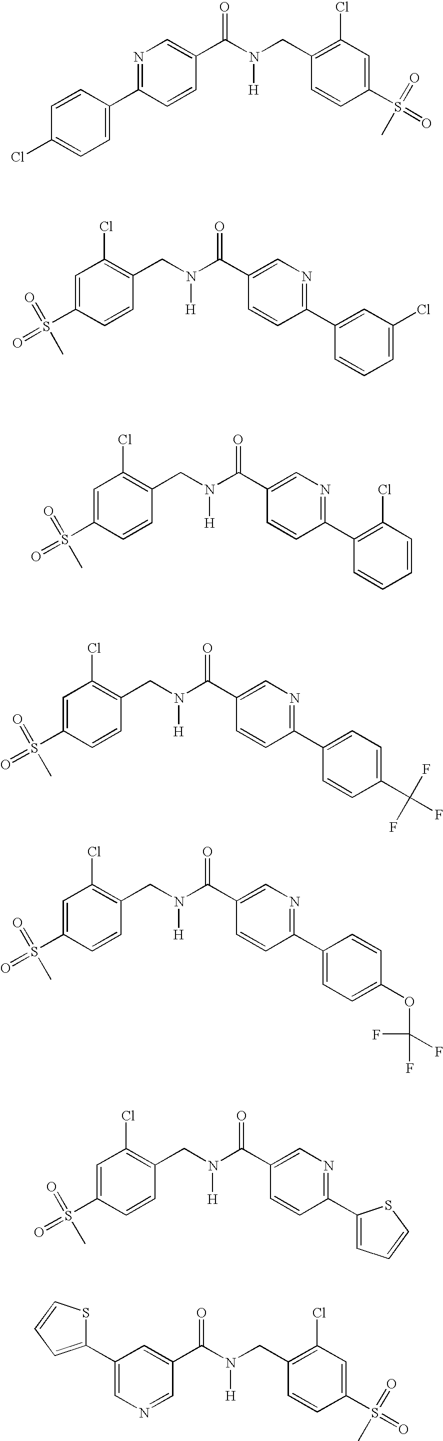

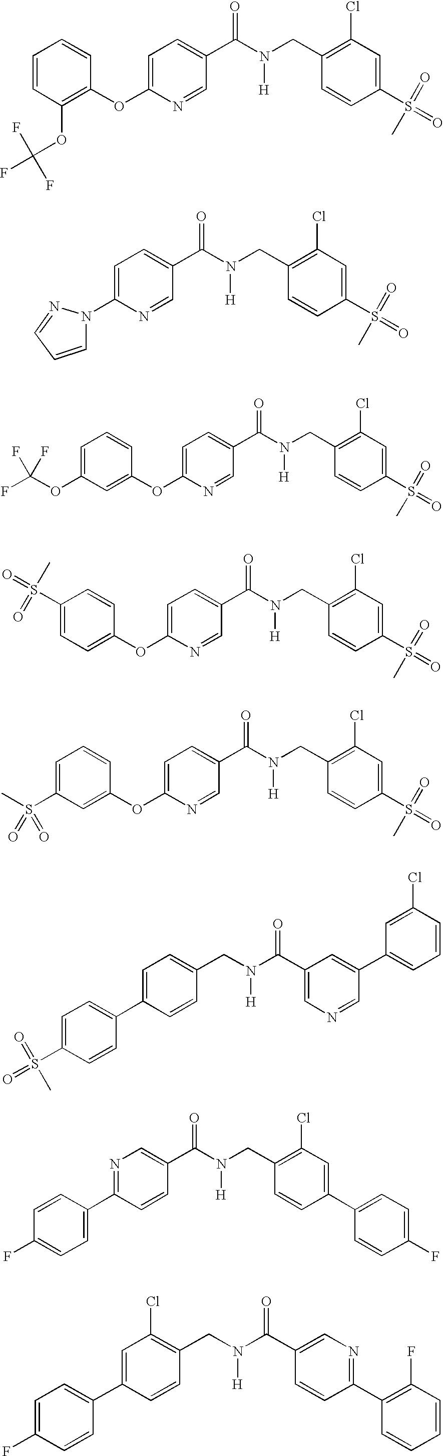

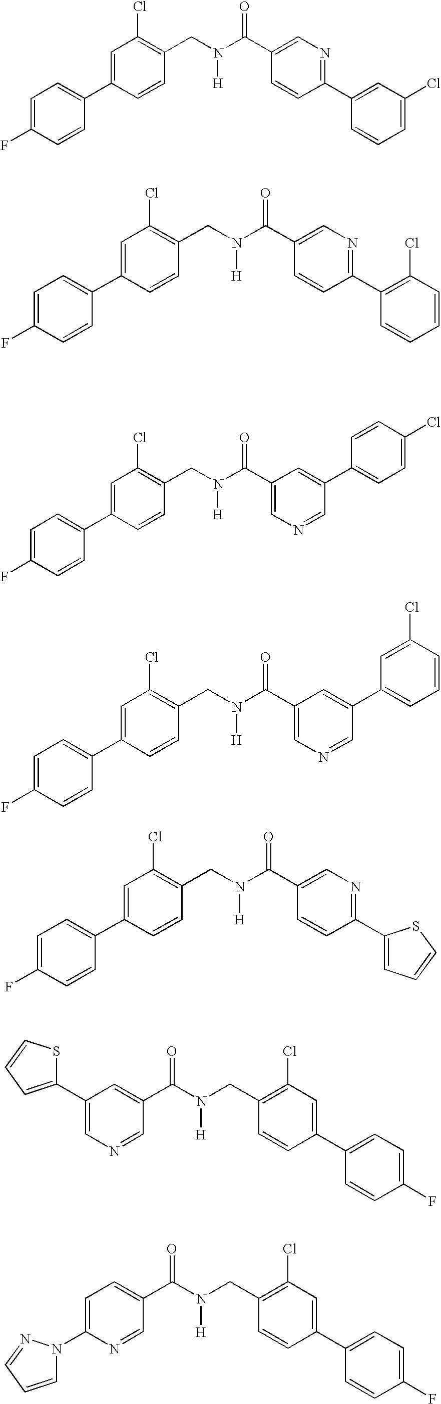

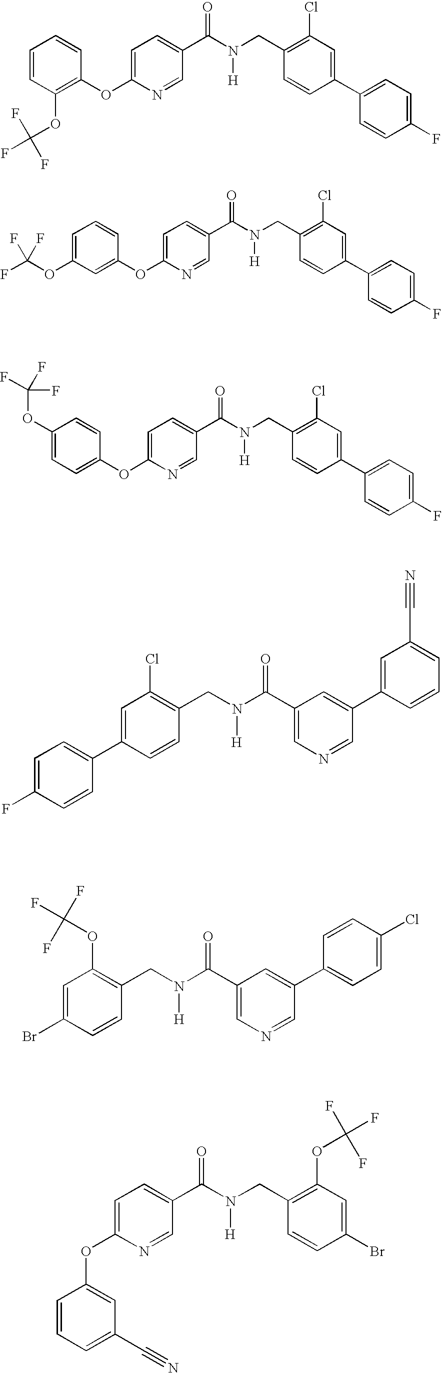

8: A method of treating a disease or condition chosen from type 1 and type 2 diabetes, insulin resistance syndrome, hypertension, atherosclerosis, coronary artery disease, angina, ischemia, ischemic stroke, Raynaud's disease and renal disease, said method comprising administering to a patient a pharmaceutically effective amount of a compound according to claim 1.

9: A pharmaceutical composition comprising a pharmaceutically effective amount of a compound according to claim 1 and one or more pharmaceutically acceptable carriers.
